(19) United States
(12) Patent Application Publication
(10) Pub. No.: US 2021/0115143 A1
(43) Pub. Date: Apr. 22, 2021

(54) ANTI-PD-L1 ANTIBODY AND USE THEREOF

(71) Applicant: R-PHARM OVERSEAS, INC., La Jolla, CA (US)

(72) Inventors: Yan LAVROVSKY, Del Mar, CA (US); Ting XU, Needham, MA (US); Sergei BARBASHOV, Irvine, CA (US); Alexey REPIK, Moscow (RU); Mikhail SAMSONOV, Moscow (RU); Vasily IGNATIEV, Moscow (RU); Shorena ARCHUADZE, Mowcow (RU)

(73) Assignee: R-PHARM OVERSEAS, INC., La Jolla, CA (US)

(21) Appl. No.: 16/606,647

(22) PCT Filed: Apr. 18, 2018
(86) PCT No.: PCT/US2018/028206

§ 371 Date: Oct. 18, 2019

(30) Foreign Application Priority Data
Apr. 18, 2017 (US) PCT/US2017/028206

(51) Int. Cl. C07K 16/28; A61P 35/00

(52) U.S. Cl. CPC C07K 16/2827; A61P 35/00; A61K 2039/505; C07K 2317/622; C07K 2317/94; C07K 2317/565

(57) ABSTRACT

Fully human anti-PD-L1 antibodies and their corresponding applications. Fully human antibodies capable of specifically binding to human PD-L1 were obtained by employing a yeast display library-based screening technique and also by affinity maturation to further improve their affinity for PD-L1. The fully human anti-PD-L1 antibodies show good specificity, affinity and stability. They are capable of enhancing T cell activity by binding to activated T cells, while significantly inhibiting tumor growth, and can be used in the diagnosis and treatment of PD-L1-related cancers and other associated diseases.

### FIELD

The present disclosure pertains to the field of biomedicine and relates to a fully human anti-PD-L1 antibodies and pharmaceutical uses thereof.

### BACKGROUND

When T cells respond to an exogenous antigen, they need antigen-presenting cells (APC) to provide two signals to resting T lymphocytes: the first signal is generated when T cells recognize antigen peptides bound to MHC molecules with the aid of TCR, after which an antigen recognition signal is transmitted via a TCR/CD3 complex; and the second signal is provided by a series of costimulatory molecules; and in this way, the T cells can be activated normally, which in turn produce a normal immune response. These costimulatory molecules can be classified as either positive costimulatory molecules or negative costimulatory molecules depending on the effects produced by the second signal, and regulation of the positive and negative costimulatory signals as well as the relative balance between said signals play an important regulatory role throughout the body's entire immune response.

PD-1 is a member of the CD28 receptor family, and said family also includes CTLA4, CD28, ICOS and BTLA. The initial members of this family, CD28 and ICOS, were discovered when monoclonal antibodies were added and observed as increasing T cell proliferation (Hutloff et al. (1999) Nature 397: 263-266; Hansen et al. (1980) Immunogenics 10: 247-260). Ligands of PD-1 include PD-L1 and PD-L2, and study results have already shown that binding of the receptor with a ligand downregulates T cell activation and the secretion of related cytokines (Freeman et al. (2000) J Exp Med 192: 1027-34; Latchman et al. (2001) Nat Immunol 2: 261-8; Carter et al. (2002) Eur J Immunol 32: 634-43; Ohigashi, et al. (2005) Clin Cancer Res 11: 2947-53).

PD-L1 (B7-H1) is a cell surface glycoprotein which belongs to the B7 family and includes IgV- and IgC-like regions, a transmembrane region and a cytoplasmic tail region. The corresponding gene was first discovered and cloned in 1999 (Dong H, et al. (1999) Nat Med 5: 1365-1369) and the glycoprotein itself was determined to interact with the T cell receptor PD-1 and play an important role in the negative regulation of the immune response. In addition to acting on PD-1 expressed on T cells, PD-L1, when expressed on T cells, can interact with CD80 on APCs to transmit negative signals, functioning as a T cell inhibitor. In addition to being expressed on macrophage lineage cells, PD-L1 is also expressed at low levels in normal human tissues, but the glycoprotein shows relatively high expression in certain tumor cell lines, including, for example, lung cancer, ovarian cancer, colon cancer and melanoma (Iwai et al. (2002) PNAS 99: 12293-7; Ohigashi, et al. (2005) Clin Cancer Res 11: 2947-53). Study results have suggested that increased expression of PD-L1 in tumor cells increases T cell apoptosis, thereby playing an important role in allowing tumor cells to evade an immune response. Researchers have found that PD-L1 gene-transfected P815 tumor cell lines can show in vitro resistance to specific CTL lysis, and said cells are more highly tumorigenic and invasive when inoculated into mice. These biological properties can be reversed by blocking PD-L1. In PD-1 knockout mice, the PD-L1/PD-1 pathway is blocked and inoculated tumor cells are unable to form tumors (Dong H et al. (2002) Nat Med 8: 793-800).

There remains a need for an anti-PD-L1 antibody which is capable of binding to PD-L1 with high affinity and thus blocking the binding of PD-1 and PD-L1.

### SUMMARY

In certain aspects of the present invention a yeast display system in conjunction with screening and affinity maturation was utilized to obtain a fully human anti-PD-L1 antibody which shows good specificity and relatively high affinity and stability, thereby completing the present invention.

The first aspect of the present invention pertains to an anti-PD-L1 antibody or an antigen-binding portion thereof, which includes a group of CDR regions selected from one of the following:

(1) heavy chain CDR1, CDR2 and CDR3 sequences which correspond to SEQ ID NO: 1-3, respectively and light chain CDR1, CDR2 and CDR3 sequences which correspond to SEQ ID NO: 4-6 respectively or sequences which are more than 70%, 80%, 85%, 90% or 95% identical to one of the aforementioned sequences, respectively;

(2) heavy chain CDR1, CDR2 and CDR3 sequences which correspond to SEQ ID NO: 7-9, respectively and light chain CDR1, CDR2 and CDR3 sequences which correspond to SEQ ID NO: 10-12 respectively or sequences which are more than 70%, 80%, 85%, 90% or 95% identical to one of the aforementioned sequences, respectively;

(3) heavy chain CDR1, CDR2 and CDR3 sequences which correspond to SEQ ID NO: 13-15, respectively and light chain CDR1, CDR2 and CDR3 sequences which correspond to SEQ ID NO: 16-18 respectively or sequences which are more than 70%, 80%, 85%, 90% or 95% identical to one of the aforementioned sequences, respectively;

(4) heavy chain CDR1, CDR2 and CDR3 sequences which correspond to SEQ ID NO: 1, 2 and 19, respectively and light chain CDR1, CDR2 and CDR3 sequences which correspond to SEQ ID NO: 4-6 respectively or sequences which are more than 70%, 80%, 85%, 90% or 95% identical to one of the aforementioned sequences, respectively;

(5) heavy chain CDR1, CDR2 and CDR3 sequences which correspond to SEQ ID NO: 7, 20 and 9, respectively and light chain CDR1, CDR2 and CDR3 sequences which correspond to SEQ ID NO: 10-12 respectively or sequences which are more than 70%, 80%, 85%, 90% or 95% identical to one of the aforementioned sequences, respectively;

(6) heavy chain CDR1, CDR2 and CDR3 sequences which correspond to SEQ ID NO: 13-15, respectively and light chain CDR1, CDR2 and CDR3 sequences which correspond to SEQ ID NO: 21, 17 and 18 respectively or sequences which are more than 70%, 80%, 85%, 90% or 95% identical to one of the aforementioned sequences, respectively.

Any one of the anti-PD-L1 antibodies or corresponding antigen-binding portions constituted by the first aspect of the present invention also includes a group of heavy chain variable region framework regions selected from one of the following:

1) FR1, FR2, FR3 and FR4 sequences which correspond to SEQ ID NO: 22-25, respectively or sequences which are more than 70%, 80%, 85%, 90%, 95% or 99% identical to one of the aforementioned sequences, respectively;

2) FR1, FR2, FR3 and FR4 sequences which correspond to SEQ ID NO: 30-33, respectively or sequences which are more than 70%, 80%, 85%, 90%, 95% or 99% identical to one of the aforementioned sequences, respectively;

3) FR1, FR2, FR3 and FR4 sequences which correspond to SEQ ID NO: 38-41, respectively or sequences which are more than 70%, 80%, 85%, 90%, 95% or 99% identical to one of the aforementioned sequences, respectively;

4) FR1, FR2, FR3 and FR4 sequences which correspond to SEQ ID NO: 30-33, respectively or sequences which are more than 70%, 80%, 85%, 90%, 95% or 99% identical to one of the aforementioned sequences, respectively.

Any one of the anti-PD-L1 antibodies or corresponding antigen-binding portions constituted by the first aspect of the present invention also includes a group of light chain variable region framework regions selected from one of the following:

1) FR1, FR2, FR3 and FR4 sequences which correspond to SEQ ID NO: 26-29, respectively or sequences which are more than 70%, 80%, 85%, 90%, 95% or 99% identical to one of the aforementioned sequences, respectively;

2) FR1, FR2, FR3 and FR4 sequences which correspond to SEQ ID NO: 30-33, respectively or sequences which are more than 70%, 80%, 85%, 90%, 95% or 99% identical to one of the aforementioned sequences, respectively;

3) FR1, FR2, FR3 and FR4 sequences which correspond to SEQ ID NO: 38-41, respectively or sequences which are more than 70%, 80%, 85%, 90%, 95% or 99% identical to one of the aforementioned sequences, respectively;

4) FR1, FR2, FR3 and FR4 sequences which correspond to SEQ ID NO: 30-33, respectively or sequences which are more than 70%, 80%, 85%, 90%, 95% or 99% identical to one of the aforementioned sequences, respectively.

Any one of the anti-PD-L1 antibodies or corresponding antigen-binding portions constituted by the first aspect of the present invention includes a group of heavy chain variable regions selected from one of the following:

1) sequences corresponding to SEQ ID NO: 47, 49, 51, 53 or 54, or a sequence which is 70%, 80%, 85%, 90%, 95% or 99% identical to one of the aforementioned sequences, respectively.

Any one of the anti-PD-L1 antibodies or corresponding antigen-binding portions thereof constituted by the first aspect of the present invention includes a group of light chain variable regions selected from the following:

1) sequences corresponding to SEQ ID NO: 48, 50, 52, 55 or 56, or a sequence which is 70%, 80%, 85%, 90%, 95% or 99% identical to one of the aforementioned sequences, respectively.

Any one of the anti-PD-L1 antibodies or corresponding antigen-binding portions constituted by the first aspect of the present invention corresponds to a whole antibody, a bispecific antibody, scFv, Fab, Fab′, F(ab′)2 or Fv.

In any example of the present invention, when the invention is constituted by an scFv, a connecting peptide is also included between the heavy chain and light chain variable regions of the aforementioned anti-PD-L1 antibody or antigen binding portion thereof.

In some specific examples of the present invention, the sequence of the aforementioned connecting peptide is as shown in SEQ ID NO: 67.

Any one example of the anti-PD-L1 antibodies or corresponding antigen-binding portions thereof constituted by the first aspect of the present invention corresponds to a whole antibody.

Any one example of the anti-PD-L1 antibodies or corresponding antigen-binding portions thereof constituted by the first aspect of the present invention, wherein the heavy chain constant region is selected from a group comprising IgG, IgM, IgE, IgD and IgA.

In certain examples of the present invention, the heavy chain constant region is selected from a group comprising IgG1, IgG2, IgG3 and IgG4.

In specific examples of the present invention, the heavy chain constant region corresponds to IgG1.

In certain specific examples of the present invention, the IgG1 amino acid sequence is as shown in SEQ ID NO: 68.

Any one of the anti-PD-L1 antibodies or corresponding antigen-binding portions constituted by the first aspect of the present invention, wherein the light chain constant region is a κ region or λ region.

In certain specific examples of the present invention, the amino acid sequence of the κ light chain constant region is as shown in SEQ ID NO: 70.

In certain specific examples of the present invention, the amino acid sequence of the λ light chain constant region is as shown in SEQ ID NO: 72.

The second aspect of the present invention pertains to a nucleic acid molecule which contains a nucleic acid sequence encoding an antibody heavy chain variable region, wherein the aforementioned antibody heavy chain variable region includes a group of amino acid sequences selected from the following:

(i) SEQ ID NO: 1-3;

(ii) SEQ ID NO: 7-9;

(iii) SEQ ID NO: 13-15;

(iv) SEQ ID NO: 1, 2 and 19;

(v) SEQ ID NO: 7, 20 and 9;

Any one of the nucleic acid molecules constituted by the second aspect of the present invention, wherein the aforementioned antibody heavy chain variable region includes a group of nucleic acid sequences which are selected from the following: SEQ ID NO: 47, SEQ ID NO: 49, SEQ ID NO: 51, SEQ ID NO: 53, SEQ ID NO: 54 or a sequence created by replacing one or several of the amino acids contained in the frame region of one of the aforementioned sequences.

In some examples of the present invention, the aforementioned nucleic acid includes a sequence selected from those shown in SEQ ID NO: 57-61.

In some examples of the present invention, the aforementioned nucleic acid also contains a nucleic acid sequence encoding an antibody heavy chain constant region, wherein said heavy chain constant region is selected from a group comprising IgG, IgM, IgE, IgD and IgA.

In some examples of the present invention, the heavy chain constant region is selected from a group comprising IgG1, IgG2, IgG3 and IgG4.

In a specific example of the present invention, the heavy chain constant region corresponds to IgG1.

In a specific example of the present invention, the IgG1 nucleic acid sequence is as shown in SEQ ID NO: 69.

The third aspect of the present invention pertains to a nucleic acid molecule which contains a nucleic acid sequence capable of encoding an antibody light chain variable region, wherein the aforementioned antibody light chain variable region includes a group of amino acid sequences selected from the following:

(i) SEQ ID NO: 4-6;

(ii) SEQ ID NO: 10-12;

(iii) SEQ ID NO: 16-18;

(iv) SEQ ID NO: 21, 17 and 18.

Any one of the nucleic acid molecules constituted by the third aspect of the present invention, wherein the aforementioned antibody light chain variable region includes a group of nucleic acid sequences which are selected from the following: SEQ ID NO: 48, SEQ ID NO: 50, SEQ ID NO: 52, SEQ ID NO: 55, SEQ ID NO: 56 or a sequence created by replacing one or several of the amino acids contained in the frame region of one of the aforementioned sequences.

In some aspects of the present invention, the aforementioned nucleic acid includes a sequence selected from those shown in SEQ ID NO: 62-66.

In some aspects of the present invention, the aforementioned nucleic acid also contains a nucleic acid sequence capable of encoding an antibody light chain constant region, wherein said light chain constant region is a κ region or λ region.

In a specific aspect of the present invention, the nucleic acid sequence of the κ light chain constant region is as shown in SEQ ID NO: 70.

In a specific aspect of the present invention, the amino acid sequence of the λ light chain constant region is as shown in SEQ ID NO: 72.

The fourth aspect of the present invention pertains to a vector which contains any one of the nucleic acids constituted by the second or third aspects of the present invention.

Any one of the vectors constituted by the fourth aspect of the present invention contains any one of the nucleic acids constituted by the second aspect of the present invention and any one of the nucleic acids constituted by the third aspect of the present invention.

The fifth aspect of the present invention pertains to a host cell which contains any one of the nucleic acids constituted by the second or third aspects of the present invention or any one of the vectors constituted by the fourth aspect of the present invention.

The sixth aspect of the present invention pertains to a conjugate which contains any one of the anti-PD-L1 antibodies or corresponding antigen-binding portions constituted by the first aspect of the present invention, as well as other biologically active substances, wherein the aforementioned anti-PD-L1 antibody or corresponding antigen-binding portion is conjugated to another biologically active substance, either directly or via a connecting fragment.

In some aspects of the present invention, the aforementioned additional biologically active substance is selected from a group comprising chemicals, toxins, polypeptides, enzymes, isotopes, cytokines or other individual biologically active substances or mixtures thereof, which are capable of directly or indirectly inhibiting cell growth or killing cells, or otherwise inhibiting or killing cells via activation of an immune response, such as Auristatin MMAE, Auristatin MMAF, Maytansine DM1, Maytansine DM4, calicheamicin, duocarmycin MGBA, doxorubicin, ricin, diphtheria toxin and other related toxins, I131, interleukins, tumor necrosis factors, chemokines, nanoparticles, etc.

The seventh aspect of the present invention pertains to a composition (such as a pharmaceutical composition), which contains any one of the anti-PD-L1 antibodies or corresponding antigen-binding portions constituted by the first aspect of the present invention, any one of the nucleic acids constituted by the second or third aspects of the present invention, any one of the vectors constituted by the fourth aspect of the present invention, any one of the host cells constituted by the fifth aspect of the present invention, or any one of the conjugates constituted by the sixth aspect of the present invention, as well as any pharmaceutically acceptable vector or excipient and any other biologically active substance(s).

Any one of the compositions constituted by the seventh aspect of the present invention (such as a pharmaceutical composition), wherein the aforementioned additional biologically active substances include, but are not limited to, other antibodies, fusion proteins or drugs (e.g., anticancer drugs, such as chemotherapy and radiotherapy drugs).

The present invention further pertains to a reagent or reagent kit which contains any one of the anti-PD-L1 antibodies or corresponding antigen-binding portions constituted by the first aspect of the present invention, wherein the aforementioned detection reagent or reagent kit is used for detecting the presence or absence of the PD-L1 protein or derivatives thereof.

The present invention further pertains to a diagnostic reagent or reagent kit which contains any one of the anti-PD-L1 antibodies or corresponding antigen-binding portions constituted by the first aspect of the present invention, wherein the aforementioned diagnostic reagent or reagent kit is used in the in vitro (e.g., cells or tissues) or in vivo (e.g., humans or model animals) diagnosis of PD-L1-related diseases (e.g., tumors or viral infections, such as cases of viral infections showing high PD-L1 expression or tumors showing high PD-L1 expression).

In some aspects of the present invention, the aforementioned anti-PD-L1 antibody or corresponding antigen-binding portion is further coupled to a fluorescent dye, chemical substance, polypeptide, enzyme, isotope, label, etc. which can be used in detection or which can be detected by a separate reagent.

In some aspects of the present invention, the aforementioned tumors include, but are not limited to, lung cancer, ovarian cancer, colon cancer, colorectal cancer, melanomas, kidney cancer, bladder cancer, breast cancer, liver cancer, lymphomas, hematologic malignancies, head and neck cancer, gliomas, gastric cancer, nasopharyngeal cancer, laryngeal cancer, cervical cancer, uterine cancer, osteosarcomas, thyroid cancer and prostate cancer.

In some aspects of the present invention, the aforementioned viral infections include, but are not limited to, acute, subacute or chronic HBV, HCV or HIV infections.

The present invention further pertains to applications of in which any one of the anti-PD-L1 antibodies or corresponding antigen-binding portions constituted by the first aspect of the present invention, any one of the nucleic acids constituted by the second or third aspects of the present invention, any one of the vectors constituted by the fourth aspect of the present invention, any one of the host cells constituted by the fifth aspect of the present invention, any one of the conjugates constituted by the sixth aspect of the present invention, or any one of the compositions constituted by the seventh aspect of the present invention is used to prepare a drug which is used in the prevention or treatment of PD-L1-related diseases (e.g., tumors or viral infections, such as cases of viral infections showing high PD-L1 expression or tumors showing high PD-L1 expression).

In certain aspects of the present invention, the aforementioned tumors refer to PD-L1-related tumors, such as tumors showing a high level of PD-L1 expression.

In specific aspects of the present invention, the aforementioned tumors include, but are not limited to, lung cancer, ovarian cancer, colon cancer, colorectal cancer, melanomas, kidney cancer, bladder cancer, breast cancer, liver cancer, lymphomas, hematologic malignancies, head and neck cancer, gliomas, gastric cancer, nasopharyngeal cancer, laryngeal cancer, cervical cancer, uterine cancer, osteosarcomas, thyroid cancer and prostate cancer.

In some aspects of the present invention, the aforementioned viral infections include, but are not limited to, acute, subacute or chronic HBV, HCV or HIV infections.

The present invention further pertains to applications in which any one of the anti-PD-L1 antibodies or corresponding antigen-binding portions constituted by the first aspect of the present invention is used to prepare a reagent or reagent kit for the diagnosis of PD-L1-related diseases (e.g., tumors or viral infections, such as cases of viral infections showing high PD-L1 expression or tumors showing high PD-L1 expression).

In some aspects of the present invention, the aforementioned tumors refer to PD-L1-related tumors, such as tumors showing a high level of PD-L1 expression.

In specific aspects of the present invention, the aforementioned tumors include, but are not limited to, lung cancer, ovarian cancer, colon cancer, colorectal cancer, melanomas, kidney cancer, bladder cancer, breast cancer, liver cancer, lymphomas, hematologic malignancies, head and neck cancer, gliomas, gastric cancer, nasopharyngeal cancer, laryngeal cancer, cervical cancer, uterine cancer, osteosarcomas, thyroid cancer and prostate cancer.

In some aspects of the present invention, the aforementioned viral infections include, but are not limited to, acute, subacute or chronic HBV, HCV or HIV infections.

In some aspects of the present invention, the aforementioned anti-PD-L1 antibody or corresponding antigen-binding portion is further coupled to a fluorescent dye, chemical substance, polypeptide, enzyme, isotope, label, etc. which can be used in detection or which can be detected by a separate reagent.

The present invention further pertains to applications in which any one of the anti-PD-L1 antibodies or corresponding antigen-binding portions constituted by the first aspect of the present invention is used to prepare a drug for the prevention or treatment of CD80-related diseases.

In the context of the present invention, the CD80-related diseases as referred to above include diseases which are related to high CD80 expression.

The present invention further pertains to a method used to prevent or treat PD-L1-related diseases (e.g., tumors or viral infections, such as cases of viral infections showing high PD-L1 expression or tumors showing high PD-L1 expression), wherein the aforementioned method includes giving a subject an effective prevention or treatment dose of any one of the anti-PD-L1 antibodies or corresponding antigen-binding portions constituted by the first aspect of the present invention, any one of the nucleic acids constituted by the second or third aspects of the present invention, any one of the vectors constituted by the fourth aspect of the present invention, any one of the host cells constituted by the fifth aspect of the present invention, any one of the conjugates constituted by the sixth aspect of the present invention, or any one of the compositions constituted by the seventh aspect of the present invention, in conjunction with the administration of optional radiotherapy (such as X-ray irradiation).

In some aspects of the present invention, the aforementioned tumors refer to PD-L1-related tumors, such as tumors showing a high level of PD-L1 expression.

In specific aspects of the present invention, the aforementioned tumors include, but are not limited to, lung cancer, ovarian cancer, colon cancer, colorectal cancer, melanomas, kidney cancer, bladder cancer, breast cancer, liver cancer, lymphomas, hematologic malignancies, head and neck cancer, gliomas, gastric cancer, nasopharyngeal cancer, laryngeal cancer, cervical cancer, uterine cancer, osteosarcomas, thyroid cancer and prostate cancer.

In some aspects of the present invention, the aforementioned viral infections include, but are not limited to, acute, subacute or chronic HBV, HCV or HIV infections.

The present invention further pertains to a method used to prevent or treat CD80-related diseases, wherein the aforementioned method includes giving a subject an effective prevention or treatment dose of any one of the anti-PD-L1 antibodies or corresponding antigen-binding portions constituted by the first aspect of the present invention.

In the context of the present invention, the CD80-related diseases as referred to above include diseases which are related to high CD80 expression.

The present invention is further described in the text below:

In the context of the present invention, unless otherwise indicated, scientific and technical terms used in this text shall corresponded to their respective common meanings as understood by a person skilled in the art. Furthermore, protein and nucleic acid chemistry, molecular biology, cell and tissue culture, microbiology and immunology-related terms, as well as laboratory procedures used in the text all correspond to terms and standard procedures which are widely employed in their respective fields. However, definitions and explanations of related terms are provided below in order to further clarify the present invention.

In the context of the present invention, the term “antibody” refers to an immunoglobulin molecule which usually consists of two pairs of identical polypeptide chains (with each pair having one “light” (L) chain and one “heavy” (H) chain). Antibody light chains may be classified as either κ or λ light chains. Heavy chains can be classified as either μ, δ, γ, α, or ε and the respective corresponding antibody isotypes are defined as being IgM, IgD, IgG, IgA, and IgE. For light and heavy chains, the variable and constant regions are connected by approximately 12 or more amino acid “J” regions, while heavy chains also contain approximately 3 or more amino acid “D” regions. Each heavy chain is composed of a heavy chain variable region (V_{H}) and a heavy chain constant region (C_{H}). The heavy chain constant region is composed of three structural domains (C_{H}1, C_{H}2 and C_{H}3). Each light chain is composed of a light chain variable region (V_{L}) and a light chain constant region (C_{L}). The light chain constant region is composed of one structural domain (C_{L}). An antibody's constant region can mediate the binding of an immunoglobulin to host tissues or factors, including the various cells of the immune system (e.g., effector cells) as well as the first component of the classical complement system (C1q). V_{H} and V_{L} regions may be further subdivided into regions with high variability (known as complementarity determining regions (CDRs)), interspersed with more conserved regions, known as framework regions (FRs). Each V_{H} and V_{L} is composed of 3 CDRs and 4 FRs which are arranged from the amino terminus to the carboxy terminus in the following order: FR1, CDR1, FR2, CDR2, FR3, CDR3 and FR4. The variable regions (V_{H} and V_{L}) of each heavy chain/light chain pair respectively form each of the antibody's binding sites. Amino acid assignment to each region or structural domain follows Kabat Sequences of Proteins of Immunological Interest (National Institutes of Health, Bethesda, Md. (1987 and 1991)) or the definition given by Chothia & Lesk (1987) J. Mol. Biol. 196: 901-917 and Chothia et al. (1989) Nature 342: 878-883. The term “antibody” is not subject to any particular limitations in terms of the method used to produce the antibody. For example, it includes, in particular, recombinant antibodies, monoclonal antibodies and polyclonal antibodies. Antibodies can be antibodies of different isotypes, including, for example, IgG (e.g., IgG1, IgG2, IgG3 or IgG4 subtypes), IgA1, IgA2, IgD, IgE, or IgM antibodies.

In the context of the present invention, the “antigen-binding portion” of an antibody refers to one or more parts along the entire length of the antibody, where said part maintains the ability to bind to the same antigen to which the antibody binds (e.g., PD-L1) and competes with intact antibodies to specifically bind to a given antigen. See generally Fundamental Immunology, Ch. 7 (Paul, W., ed., 2nd edition, Raven Press, NY (1989), which is for all purposes incorporated herein via full-text citation. Antigen-binding portions can be produced via recombinant DNA techniques or via the enzymatic or chemical breakdown of whole antibodies. In some instances, the antigen binding portion includes a Fab, Fab′, F(ab′)₂, Fd, Fv, dAb, complementarity determining region (CDR) fragment, single chain antibody (e.g., scFv), chimeric antibody, diabody and similar polypeptides, which include at least a portion of an antibody which is capable of imparting a polypeptide-specific antigen binding capacity.

In the context of the present invention, the term “Fd fragment” refers to an antibody fragment consisting of V_{H} and C_{H}1 structural domains; the term “Fv fragment” refers to an antibody fragment consisting of the V_{L} and V_{H} structural domains of the single arm of an antibody; the term “dAb fragment” refers to an antibody fragment composed of a V_{H} structural domain (Ward et al., Nature 341: 544-546 (1989)); the term “Fab fragment” refers to an antibody fragment composed of V_{L}, V_{H}, C_{L} and C_{H}1 structural domains; and the term “F(ab′)₂ fragment” refers to an antibody fragment which includes two Fab fragments which are connected via a disulfide bridge in the hinge region.

In some cases, the antigen-binding portion of the antibody is a single chain antibody (e.g., scFv), where the V_{L} and V_{H} structural domains form a monovalent molecule via pairing by allowing it to be produced as a single polypeptide chain linker (see, for example, Bird et al., Science 242: 423-426 (1988) and Huston et al., Proc. Natl. Acad. Sci. USA 85: 5879-5883 (1988)). Such an scFv molecule can have the general structure of: NH₂—V_{L}-connector-V_{H}—COOH or NH₂—V_{H}-connector-V_{L}—COOH. Suitable conventional connectors (connecting peptides) are composed of repeating GGGGS amino acid sequences or variants thereof. For example, a connector with the amino acid sequence (GGGGS)₄ can be used, but variants can also be used (Holliger et al. (1993), Proc. Natl. Acad. Sci. USA 90: 6444-6448). Other connectors which can be used for the present invention are described in Alfthan et al. (1995), Protein Eng. 8: 725-731, Choi et al. (2001), Eur. J. Immunol. 31: 94-106, Hu et al. (1996), Cancer Res. 56: 3055-3061, Kipriyanov et al. (1999), J. Mol. Biol. 293: 41-56 and Roovers et al. (2001), Cancer Immunol. In an aspect of the present invention, the sequence of the aforementioned connecting peptide is (GGGGS)₃.

In some instances, the antibody is constituted by a bispecific antibody which is capable of respectively binding two different kinds of antigen or antigenic epitope and which includes a light chain and heavy chain of an antibody which specifically binds to a primary antigen, or an antigen-binding portion thereof, as well as a light chain and heavy chain of an antibody which specifically binds to a secondary antigen, or an antigen-binding portion thereof. In some aspects of the present invention, the light chain and heavy chain of an antibody which specifically binds to a primary antigen, or an antigen-binding portion thereof, included in the aforementioned bispecific antibody can correspond to any one of the antibodies or corresponding antigen-binding portions constituted by the present invention, and the light chain and heavy chain of an antibody which specifically binds to a secondary antigen, or an antigen-binding portion thereof, included in the aforementioned bispecific antibody can correspond to a different anti-PD-L1 antibody or corresponding antigen-binding portion, or an antibody targeting a different antigen or corresponding antigen-binding portion.

In some cases, the antibodies correspond to diabodies, i.e., bivalent antibodies, wherein V_{H} and V_{L} structural domains are expressed on a single polypeptide chain, but a linker which is too short is used, which does not allow pairing between the two structural domains on the same chain, thereby forcing the structural domains to pair with complementary structural domains of another chain and creating two antigen binding sites (see, for example, Holliger P. et al., Proc. Natl. Acad. Sci. USA 90: 6444-6448 (1993), and Poljak R. J. et al., Structure 2: 1121-1123 (1994)).

Conventional techniques known by persons skilled in the art (e.g., recombinant DNA techniques or enzymatic or chemical cleavage) can be used to obtain the antigen-binding portion (e.g., an antibody fragment as described above) from a given antibody (such as the monoclonal antibody 2E12), and selectively screen for antigen-binding portions of the antibody using the same methods as those used for whole antibodies.

In the context of the present invention, the antigen binding portions as referred to above include single chain antibodies (scFv), chimeric antibodies, diabodies, scFv-Fc bivalent molecules, dAb and complementarity determining region (CDR) fragments, Fab fragments, Fd fragments, Fab′ fragments and Fv and F(ab′)₂ fragments.

In the context of the present invention, IgG1 heavy chain constant regions as referred to above include allotypes such as G1m(f), G1m(z), G1m(z,a) and G1m(z,a,x). In some aspects of the present invention, the aforementioned IgG1 heavy chain constant region corresponds to G1m(f).

In the context of the present invention, the aforementioned κ light chain constant region includes various allotypes, such as Km1, Km1,2 and Km3. In some aspects of the present invention, the aforementioned κ light chain constant region corresponds to a Km3 type region.

In the context of the present invention, the aforementioned λ light chain constant region includes various allotypes, such as λI, λII, λIII and λVI. In some aspects of the present invention, the aforementioned λ light chain constant region corresponds to a λII type region.

Antibody nucleic acids to which the present invention pertains can also be obtained via conventional genetic engineering recombinant techniques or chemical synthesis methods. On the one hand, the sequences of antibody nucleic acids to which the present invention pertains include anti-PD-L1 antibody heavy chain variable regions or partial nucleic acid sequences belonging to antibody molecules. On the other hand, the sequences of antibody nucleic acids to which the present invention pertains also include anti-PD-L1 antibody light chain variable regions or partial nucleic acid sequences belonging to antibody molecules. On yet another hand, the sequences of antibody nucleic acids to which the present invention pertains furthermore also include CDR sequences belonging to the heavy chain and light chain variable regions. The complementarity determining region (CDR) is a site which binds to an antigen epitope and, within the context of the present invention, CDR sequences are verified via IMGT/V-QUEST (http://imgt.cines.fr/textes/vquest/). However, CDR sequences obtained via different parsing methods are slightly different.

One aspect of the present invention pertains to nucleic acid molecules which code for antibody B60-55, BII61-62, B50-6, B60, BII61 and B50 heavy and light chain variable region sequences. Nucleic acid molecules which code for antibody B60-55, BII61-62, B50-6, B60, BII61 and B50 heavy chain variable region sequences correspond to SEQ ID NO: 57, SEQ ID NO: 58, SEQ ID NO: 59, SEQ ID NO: 60, SEQ ID NO: 61 and SEQ ID NO: 59, respectively. Nucleic acid molecules which code for antibody B60-55, BII61-62, B50-6, B60, BII61 and B50 light chain variable region sequences correspond to SEQ ID NO: 62, SEQ ID NO: 63, SEQ ID NO: 64, SEQ ID NO: 62, SEQ ID NO: 65 and SEQ ID NO: 66, respectively. The present invention also pertains to variants or analogs of nucleic acid molecules which code for antibody B60-55, BII61-62, B50-6, B60, BII61 and B50 heavy and light chain variable region sequences.

On the other hand, the present invention also pertains to various separated nucleic acid molecule variants; specifically, the sequence of said nucleic acid variants should show at least 70% similarity with the following nucleic acid sequences: SEQ ID NO: 57, SEQ ID NO: 58, SEQ ID NO: 59, SEQ ID NO: 60, SEQ ID NO: 61, SEQ ID NO: 59, SEQ ID NO: 62, SEQ ID NO: 63, SEQ ID NO: 64, SEQ ID NO: 62, SEQ ID NO: 65 and SEQ ID NO: 66, with a similarity reaching at least 75% being preferable, similarity reaching at least 80% being more preferable, similarity reaching at least 85% being even more preferable, similarity reaching at least 90% being yet even more preferable and similarity reaching at least 95% being most preferable.

The present invention further pertains to corresponding separated nucleic acid molecules which code for antibody B60-55, BII61-62, B50-6, B60, BII61 and B50 heavy chain variable region sequences in the form of the amino acid sequences SEQ ID NO: 47, 49, 51, 53, 54 and 51. The present invention also pertains to corresponding nucleic acid molecules which code for antibody B60-55, BII61-62, B50-6, B60, BII61 and B50 light chain variable region sequences in the form of the amino acid sequences SEQ ID NO: 48, 50, 52, 48, 55 and 56.

The present invention pertains to a recombinant expression vector which contains the aforementioned nucleic acid molecules and furthermore pertains to a host cell which has been transformed with said molecules. Furthermore, the present invention pertains to methods which are used to culture host cells which contain the aforementioned nucleic acid molecules under specific conditions, followed by separation to obtain antibodies as described by the invention.

Antibody Amino Acid Sequences

The amino acid sequences of monoclonal antibody mAb B60-55, BII61-62, B50-6, B60, BII61 and B50 heavy and light chain variable regions may be derived from the corresponding nucleic acid sequences. The amino acid sequences of the antibody mAb B60-55, BII61-62, B50-6, B60, BII61 and B50 heavy chain variable regions correspond to SEQ ID NO: 47, 49, 51, 53, 54 and 51, respectively. The amino acid sequences of the antibody mAb B60-55, BII61-62, B50-6, B60, BII61 and B50 light chain variable regions correspond to SEQ ID NO: 48, 50, 52, 48, 55 and 56, respectively.

On the other hand, the amino acid sequences of the heavy chain variable regions of antibodies provided by the present invention should show at least 70% similarity with the sequences given in SEQ ID NO: 47, 49, 51, 53, 54 and 51, with similarity reaching at least 80% being preferable, similarity reaching at least 85% being more preferable, similarity reaching at least 90% being even more preferable and similarity reaching at least 95% being most preferable.

On the other hand, the amino acid sequences of the light chain variable regions of antibodies provided by the present invention should show at least 70% similarity with the sequences given in SEQ ID NO: 48, 50, 52, 48, 55 and 56, with similarity reaching at least 80% being preferable, similarity reaching at least 85% being more preferable, similarity reaching at least 90% being even more preferable and similarity reaching at least 95% being most preferable.

The CDR amino acid sequences for the heavy and light chain variable regions of the antibodies B60-55, BII61-62, B50-6, B60, BII61 and B50 are determined as follows:

The amino acid sequences for CDR1, CDR2 and CDR3 of the heavy chain of the antibody B60-55 correspond to SEQ ID NO: 1-3, respectively. The amino acid sequences for CDR1, CDR2 and CDR3 of the light chain of the antibody B60-55 correspond to SEQ ID NO: 4-6, respectively.

The amino acid sequences for CDR1, CDR2 and CDR3 of the heavy chain of the antibody BII61-62 correspond to SEQ ID NO: 7-9, respectively. The amino acid sequences for CDR1, CDR2 and CDR3 of the light chain of the antibody BII61-62 correspond to SEQ ID NO: 10-12, respectively.

The amino acid sequences for CDR1, CDR2 and CDR3 of the heavy chain of the antibody B50-6 correspond to SEQ ID NO: 13-15, respectively. The amino acid sequences for CDR1, CDR2 and CDR3 of the light chain of the antibody B50-6 correspond to SEQ ID NO: 16-18, respectively.

On the other hand, an amino acid sequence contained in the CDR of the heavy chain of an anti-PD-L1 antibody or fragment thereof may be obtained via one or more amino acid mutations, additions or deletions of SEQ ID NO: 1-3, 7-9, 13-15, 19 and 20. Preferably, the number of amino acids subject to mutation, addition or deletion should not exceed three. More preferably, the number of amino acids subject to mutation, addition or deletion should not exceed two. Most preferably, the number of amino acids subject to mutation, addition or deletion should not exceed one.

On the other hand, an amino acid sequence contained in the CDR of the light chain of an anti-PD-L1 antibody or fragment thereof may be obtained via one or more amino acid mutations, additions or deletions of SEQ ID NO: 4-6, 10-12, 16-18 and 21. Preferably, the number of amino acids subject to mutation, addition or deletion should not exceed three. More preferably, the number of amino acids subject to mutation, addition or deletion should not exceed two. Most preferably, the number of amino acids subject to mutation, addition or deletion should not exceed one.

The FR amino acid sequences for the heavy and light chain variable regions of the antibodies B60-55, BII61-62, B50-6, B60, BII61 and B50 are determined as follows:

The FR1, FR2, FR3 and FR4 sequences of the heavy chain variable regions of the antibodies B60-55 and B60 correspond to SEQ ID NO: 22-25, respectively. The FR1, FR2, FR3 and FR4 sequences of the light chain variable regions correspond to SEQ ID NO: 26-29, respectively.

The FR1, FR2, FR3 and FR4 sequences of the heavy chain variable regions of the antibody BII61-62 correspond to SEQ ID NO: 30-33, respectively. The FR1, FR2, FR3 and FR4 sequences of the light chain variable regions correspond to SEQ ID NO: 34-37, respectively.

The FR1, FR2, FR3 and FR4 sequences of the heavy chain variable regions of the antibodies B50-6 and B50 correspond to SEQ ID NO: 38-41, respectively. The FR1, FR2, FR3 and FR4 sequences of the light chain variable regions correspond to SEQ ID NO: 42-45, respectively.

The FR1, FR2, FR3 and FR4 sequences of the heavy chain variable regions of the antibody BII61 correspond to SEQ ID NO: 30-33, respectively. The FR1, FR2, FR3 and FR4 sequences of the light chain variable regions correspond to SEQ ID NO: 34, 46, 36, 37, respectively.

On the other hand, an amino acid sequence contained in the FR of the heavy chain variable region of an anti-PD-L1 antibody may be obtained via one or more amino acid mutations, additions or deletions of SEQ ID NO: 22-46. Preferably, the number of amino acids subject to mutation, addition or deletion should not exceed three. More preferably, the number of amino acids subject to mutation, addition or deletion should not exceed two. Most preferably, the number of amino acids subject to mutation, addition or deletion should not exceed one.

Variants which are obtained following the mutation, addition or deletion of an amino acid contained in an aforementioned antibody, CDR or frame region should still retain the ability to bind specifically to human PD-L1. The present invention also includes such variants of the antigen-binding portion.

A variant of aforementioned antibodies is antibody B60-55-1 which has a complete heavy chain of SEQ ID NO: 85 and a complete light chain of SEQ ID NO: 87, the terminal lysine residue at the C-terminus of the heavy chain may be missing. The heavy chain of B60-55-1 can be expressed by utilizing a nucleic acid sequence of SEQ ID NO: 86. The nucleic acid sequence can be incorporated into an expression vector for further incorporation into an expression cell line. The light chain of B60-55-1 can be expressed by utilizing a nucleic acid sequence of SEQ ID NO: 88. The nucleic acid sequence can be incorporated into an expression vector for further incorporation into an expression cell line.

B60-55-1 antibody can be formulated as a pharmaceutical composition by adding a pharmaceutically acceptable excipient or adjuvant. The composition may contain about 275 mM serine, about 10 mM histidine, and have a pH value of about 5.9. The composition may contain about 0.05% polysorbate 80, about 1% D-mannitol, about 120 mM L-proline, about 100 mM L-serine, about 10 mM L-histidine-HCl, and having a pH of about 5.8.

Monoclonal antibody variants constituted by the present invention can be obtained by conventional genetic engineering methods. Those skilled in the art are fully aware of methods which employ nucleic acid mutation to modify DNA molecules. Additionally, nucleic acid molecules which code for heavy chain and light chain variants can also be obtained via chemical synthesis.

In the context of the present invention, examples of algorithms which are used to determine the sequence identity and sequence similarity percentage include BLAST and BLAST 2.0, which are described in Altschul et al. (1977) Nucl. Acid. Res. 25: 3389-3402 and Altschul et al. (1990) J. Mol. Biol. 215: 403-410, respectively. Using, for example, parameters given in the literature or the default parameters, BLAST and BLAST 2.0 can be used to determine the percentage similarity of amino acid sequences constituted by the present invention. Software capable of performing a BLAST analysis can be obtained by any member of the public via the National Center for Biotechnology Information.

In the context of the present invention, amino acid sequences which are at least 70% identical to a given amino acid sequence as stated above include polypeptide sequences which are fundamentally identical to said amino acid sequence, such as sequences which are determined to be at least 70% identical to a polypeptide sequence constituted by the present invention when methods outlined in this text (e.g., BLAST analysis employing standard parameters) are used, with sequences showing at least 75%, 80%, 85%, 86%, 87%, 88%, 89%, 90%, 91%, 92%, 93%, 94%, 95%, 96%, 97%, 98%, 99% or greater preferred.

In the context of the present invention the term “vector” refers to a type of nucleic acid delivery vehicle which includes a polynucleotide coding for a certain protein and which allows said protein to be expressed. A vector allows for expression of the genetic material component(s) which it carries within a host cell following transformation, transduction or transfection of said host cell. For example, the vectors include: plasmids; phagemids; cosmids; artificial chromosomes such as a yeast artificial chromosome (YAC), bacterial artificial chromosome (BAC) or a P1-derived artificial chromosome (PAC); bacteriophages such as a λ phage or M13 phage and animal viruses. Examples of animal viruses used as a vector include retroviruses (including lentiviruses), adenoviruses, adeno-associated viruses, herpes viruses (such as the herpes simplex virus), poxviruses, baculoviruses, papilloma viruses and papova viruses (e.g., SV40). A vector may contain several expression control elements, including promoter sequences, transcription initiation sequences, enhancer sequences, selection elements and reporter genes. Furthermore, the vector may contain an origin of replication. Vectors may also include components which facilitate entry into a cell, such as viral particles, liposomes or a protein coat, but said components are not limited to the above substances.

In the context of the present invention, the term “host cell” refers to a cell into which a vector is introduced, comprising a number of different cell types, including prokaryotic cells such as E. coli or B. subtilis, fungal cells such as yeast cells or Aspergillus, insect cells such as Drosophila S2 cells or Sf9, or animal cells such as fibroblasts, CHO cells, COS cells, NS0 cells, HeLa cells, BHK cells, HEK 293 cells or other human cells.

Antibody fragments constituted by the present invention can be obtained via hydrolysis of whole antibody molecules (see Morimoto et al., J. Biochem. Biophys. Methods 24: 107-117 (1992) and Brennan et al., Science 229: 81 (1985)). Additionally, these antibody fragments can also be directly produced by recombinant host cells (reviewed in Hudson, Curr. Opin. Immunol. 11: 548-557 (1999); Little et al., Immunol. Today, 21: 364-370 (2000)). For example, Fab′ fragments can be directly obtained from E. coli cells or chemically coupled to form F(ab′)₂ fragments (Carter et al., Bio/Technology, 10: 163-167 (1992)). As another example, F(ab′)₂ fragments can be obtained via connection using the GCN4 leucine zipper. Additionally, Fv, Fab or F(ab′)₂ fragments can also be directly isolated from a recombinant host cell culture medium. An ordinary person skilled in the art would be fully aware of other techniques for the production of antibody fragments.

In the context of the present invention, the term “specific binding” refers to a non-random binding reaction between two molecules, such as a reaction occurring between an antibody and a corresponding antigen. Here, the binding affinity of an antibody which binds a primary antigen for a secondary antigen is very weak or undetectable. In certain aspects, an antibody which is specific for a given antigen binds said antigen with an affinity (KD) of ≤10⁻⁵ M (e.g., 10⁻⁶ M, 10⁻⁷ M, 10⁻⁸ M, 10⁻⁸ M or 10⁻¹° M), where KD refers to the ratio of the dissociation rate to the binding rate (koff/kon) and this quantity can be measured via methods familiar to a person skilled in the art.

In some aspects of the present invention, an anti-PD-L1 antibody constituted by the present invention is capable of specifically binding to human PD-L1 and simultaneously also binding to murine PD-L1, but does not bind to PD-L2 or B7H3. In some aspects of the present invention, an anti-PD-L1 antibody constituted by the present invention is capable of binding hPD-L1 competitively with respect to hPD-1.

In the context of the present invention, PD-L1-related diseases include, for example, tumors and viral infections which are linked to PD-L1, particularly tumors and viral infections which are associated with a high level of PD-L1 expression.

In some aspects of the present invention, the aforementioned tumors include, but are not limited to, lung cancer, ovarian cancer, colon cancer, colorectal cancer, melanomas, kidney cancer, bladder cancer, breast cancer, liver cancer, lymphomas, hematologic malignancies, head and neck cancer, gliomas, gastric cancer, nasopharyngeal cancer, laryngeal cancer, cervical cancer, uterine cancer, osteosarcomas, thyroid cancer and prostate cancer.

In some aspects of the present invention, aforementioned viral infections include, but are not limited to, acute, subacute or chronic HBV, HCV or HIV infections.

In the context of the present invention, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein via citation.

### Benefits of the Invention

The invention employs yeast display technology in conjunction with screening and affinity maturation to obtain a fully human anti-PD-L1 antibody which shows good specificity and relatively high affinity and stability, wherein said antibody is capable of specifically binding to human PD-L1 or simultaneously also binding to murine PD-L1 and does not bind to B7H3 or PD-L2; and said antibody binds to activated T cells to further enhance T cell activation and produces significant inhibition of tumor growth.

### BRIEF DESCRIPTION OF THE DRAWINGS

FIG. 1: Inhibition of hPD-L1/hPD-1 ligand-receptor binding by purified anti-hPD-L1 scFv.

The X-axis represents the EGFP fluorescence intensity while the Y-axis represents the SA-PE fluorescence intensity. A—corresponds to a blank control, B—corresponds to a negative control, C—corresponds to B50 scFv, D—corresponds to B60 scFv and E corresponds to BII61 scFv.

FIG. 2: Yeast showing increased affinity for hPD-L1 yeast following affinity maturation screening

Here, the X-axis represents the fluorescence intensity of myc (myc-positive corresponding to yeast expressing whole antibody fragments) and the Y-axis represents the fluorescence intensity SA-APC, which indicates the antigen binding ability.

FIG. 3: A comparison of the ability of antibodies obtained following affinity maturation to bind hPD-L1 in competition with hPD-1

Here, the horizontal axis corresponds to the antibody concentration (units: ng/ml) and the vertical axis corresponds to the OD value.

A) shows a comparison of BII61-62 and BII61, B) shows a comparison of B50 and B50-6 and C) shows a comparison of B60 and B60-55.

FIG. 4: ELISA measurements of anti-hPD-L1 antibody and hPD-L1 binding capacity

Here, the horizontal axis corresponds to the antibody concentration (units: ng/ml) and the vertical axis corresponds to the OD value.

FIG. 5: Competitive ELISA measurement of anti-hPD-L1 and hPD-1 competitive binding of hPD-L1

Here, the horizontal axis corresponds to the antibody concentration (units: ng/ml) and the vertical axis corresponds to the OD value.

Graph #5 corresponds to BII61-62 mAb, Graph #2 corresponds to B50-6 mAb and Graph #3 corresponds to B60-55 mAb.

FIG. 6: Competitive ELISA measurement of anti-hPD-L1 and CD80 competitive binding of hPD-L1

FIG. 7: Detection of anti-hPD-L1 antibody specificity

Here, the X-axis represents the EGFP fluorescence intensity, the Y-axis represents the fluorescence intensity of the corresponding antibody binding, A—corresponds to a blank control, B—corresponds to a negative control, C—corresponds to BII61-62 mAb, D—corresponds to B60-55 mAb and E—corresponds to B50-6 mAb;

(1) corresponds to a hPD-L1-EGFP protein, (2) corresponds to hB7H3-EGFP and (3) corresponds to a hPD-L2-EGFP protein.

FIG. 8: Anti-hPD-L1 antibody and mPD-L1 binding capacity

Here, the X-axis represents the EGFP fluorescence intensity, the Y-axis represents the fluorescence intensity of the corresponding antibody binding, A—corresponds to a blank control, B—corresponds to a negative control, C—corresponds to B60-55 mAb, D—corresponds to BII61-62 mAb and E corresponds to B50-6 mAb;

(1) corresponds to a hPD-L1-EGFP protein and (2) corresponds to a mPD-L1-EGFP protein.

FIG. 9: Anti-hPD-L1 antibody and cynomolgus monkey PD-L1 binding capacity FIG. 10: Activation of CD4⁺T cells by anti-hPD-L1 antibodies

FIG. 11: Inhibitory activity of the anti-hPD-L1 antibody B50-6 on tumor growth

FIG. 12: Inhibitory activity of the anti-hPD-L1 antibodies B60-55 and BII61-62 on tumor growth

Here, A—corresponds to BII61-62 mAb and B60-55 inhibition of tumor growth when a dose of 3 mg/kg is used; and B—corresponds to the inhibitory effects of BII61-62 mAb on tumor growth when different dosages are used.

FIG. 13: A comparison of the stability of B60-55 and the antibody 2.41H90P

Here, A—corresponds to the IC50 values of B60-55 and the antibody 2.41H90P over time; B—corresponds to the proportion of antibody dimers over time; and C—corresponds to the competitive ELISA results obtained in B60-55 accelerated stability testing.

FIG. 14: Chromatography of B60-55-1 on CaPure-HA; B60-55-1 retention time is about 45 min.

FIG. 15: Size exclusion chromatography analysis of purified B60-55-1 on TSKgel G3000SW_{XL}(Tosoh) column.

FIG. 16: Coomassie stained SDS-PAGE analysis of purified B50-55-1: lane 1—under reduced conditions, lane 2—under non-reducing conditions, lane 3—molecular weight markers.

FIG. 17: Alternative capturing approaches for SPR measurements:

Panel A—Anti-human-IgG was immobilized on the chip as capturing antibodies; B60-55-1 or atezolizumab were captured by immobilized antibodies and various concentrations of PD-L1-His ligand were applied.

Panel B—PD-L1-Fc fusion protein was directly immobilized on the sensor chip and different concentrations of B60-55-1 or atezolizumab were applied.

Panel C—to study interactions with both PD-L1-Fc fusion protein and PD-L1-His, B60-55-1 or atezolizumab were directly immobilized on the chip; a range of concentrations of PD-L1-His tagged or PD-L1-Fc were applied.

FIG. 18: Sensograms of binding of PD-L1-His tagged ligand to immobilized comparator antibody atezolizumab or B60-55-1; the approach is schematically shown in the left panel and kinetic parameters are summarized in the table; anti-human capturing antibodies were immobilized on a sensor chip and atezolizumab or B60-55-1 were captured then followed by various concentrations of PD-L1-His ligand:

Panel A—results for atezolizumab;

Panel B—results for B60-55-1.

FIG. 19: Sensograms of binding of atezolizumab or B60-55-1 to immobilized PD-L1-Fc fusion protein; the approach is schematically shown on the left panel and kinetic parameters are summarized in the table; various concentrations of B60-55-1 or atezolizumab were applied to the chip:

Panel A—results for atezolizumab;

Panel B—results for B60-55-1.

FIG. 20: Sensograms of binding of PD-L1-His or PD-L1-Fc to immobilized B60-55-1; the approach is schematically shown in the left panel and kinetic parameters are summarized in the table.

FIG. 21: Sensograms of binding of PD-L1-His or PD-L1-Fc to immobilized atezolizumab; the approach is schematically shown in left panel and kinetic parameters are summarized in the table.

FIG. 22: B60-55-1 and atezolizumab have no ADCC activities compared to the control antibodies from the Promega ADCC Reporter Bioassay Kit.

FIG. 23: Evaluation of B60-55-1 and atezolizumab binding to C1q.

FIG. 24: Concentration dependent potencies of B60-55-1 and comparator antibodies on T cell activation in MLR assay.

FIG. 25: Body weight change upon drug treatment; arrows indicated the dosing time.

FIG. 26: Tumor volume inhibition upon drug treatment; arrows indicated the dosing time.

FIG. 27: Individual tumor growth in three groups during 29 days' observation after grouping (n=8).

FIG. 28: Tumor weight inhibition at day 29 posting dosing.

FIG. 29: Mean tumor volume in the three test groups from experimental design shown in Table 7 below.

FIG. 30: Mean tumor volume in the three test groups from experimental design shown in Table 7 below at days 21 and 41; three columns for each day correspond to group 1 (left), two (center) and 3 (right).

### DETAILED DESCRIPTION

In the following section, the aspects of the present invention are further illustrated via the following examples; however, as should be understood by any person skilled in the art, the following examples are only used to illustrate the present invention and should not be construed as limiting the scope of the present invention. Any conditions which are not specified in the following examples should be set to be those used conventionally or those recommended by the manufacturer. Any reagents or instruments used for which a manufacturer is not specified all correspond to standard products which can be purchased commercially.

### Example 1: Recombinant Human PD-L1 and PD-1 Expression and Preparation of Related EGFP Cells

The amino acid sequence of the extracellular domain of human PD-L1 was obtained based on an amino acid sequence of PD-L1 (Q9NZQ7) contained in the protein database Uniprot (i.e., the sequence from Residue 1 to Residue 238 contained in Q9NZQ7); the amino acid sequence of the structural domain of IgG1-Fc was obtained based on an amino acid sequence of the constant region of human immunoglobulin gamma1 (IgG1) (P01857) contained in the protein database Uniprot (i.e., the sequence from Residue 104 to Residue 330 contained in P01857); and the amino acid sequence of the structural domain of IgG1-Fc was obtained based on an amino acid sequence of the constant region of human immunoglobulin gamma1 (IgG1) (P01868) contained in the protein database Uniprot (i.e., the sequence from Residue 98 to Residue 324 contained in P01868). The online tool DNAworks (http://helixweb.nih.gov/dnaworks/) was used to design corresponding encoding DNA sequences to obtain hPD-L1-Fc and hPD-L1-muFc fusion protein genes, and the same method was used to obtain a hPD-1-Fc gene. An amino acid sequence for enhanced green fluorescent protein (EGFP) (C5MKY7) as well as an amino acid sequence for human PD-L1 (Q9NZQ7), an amino acid sequence for murine PD-L1 (Q9EP73) and an amino acid sequence for human PD-1 (Q15116) were obtained based on information contained in the protein database Uniprot. The online tool DNAworks (http://helixweb.nih.gov/dnaworks/) was used to design corresponding encoding DNA sequences to obtain a PD-L1-EGFP fusion protein gene, and the same method was used to obtain hPD-1-EGFP and mPD-L1-EGFP genes. Corresponding DNA fragments were obtained via artificial synthesis. Synthesized gene sequences were double digested with Fermentas-made HindIII and EcoRI and cloned into the commercial vector pcDNA4/myc-HisA (Invitrogen, V863-20), after which sequencing was performed to verify that the plasmid had been constructed accurately to obtain recombinant plasmid DNA; i.e.: pcDNA4-hPD-L1-Fc, pcDNA4-hPD-L1-muFc, pcDNA4-hPD1-Fc, pcDNA4-hPD-L1-EGFP, pcDNA4-hPD1-EGFP and pcDNA4-mPD-L1-EGFP.

Reverse transcription-polymerase chain reaction (RT-PCR) was used to amplify human PD-L2 and B7H3 genes from lab-cultured dendritic cells (DC cells) (wherein said DC cells were obtained via TNF-α maturation of mononuclear cells isolated from PBMC) and the gene amplification primers used were as follows:

PDL2-F HindIII:
(SEQ ID NO: 74)
GCGCAAGCTTGCCACCATGATCTTCCTCCTGCTAATG,
PDL2-R EcoI:
(SEQ ID NO: 75)
GCCGAATTCGATAGCACTGTTCACTTCCCTC;
hB7H3-F HindIII:
(SEQ ID NO: 76)
GCGCAAGCTTGCCACCATGCTGCGTCGGCGGGGCAGC;
hB7H3-R BamHI:
(SEQ ID NO: 77)
GCGCGAATTCGGCTATTTCTTGTCCATCATCTTC.

The PCR product obtained was then double digested using Fermentas HindIII and EcoRI and cloned into a pre-constructed pcDNA4-hPD-L1-EGFP, after which sequencing was performed to verify that the plasmid had been constructed accurately to obtain recombinant plasmid DNA; i.e.: pcDNA4-hPD-L2-EGFP and pcDNA4-hB7H3-EGFP.

A corresponding EGFP recombinant plasmid was transfected into HEK293 (ATCC, CRL-1573™) cells, and fluorescence-activated cell sorting (FACS) was performed 48 hours after transfection to verify the expression of hPD-L1, mPD-L1, hPD-L2 and hB7H3.

pcDNA4-hPD-L1-Fc, pcDNA4-hPD-L1-muFc and pcDNA4-hPD1-Fc were transiently transfected into HEK293 cells for protein production. The recombinant expression plasmid was diluted with a Freestyle293 culture medium and added to a PEI (polyethylenimine) solution required for transformation, after which each plasmid/PEI mixture was each separately added to a cell suspension and left to culture at 37° C. 10% CO₂ and 90 rpm; at the same time, a supplementary addition of 50 μg/L insulin-like growth factor (IGF-1) was performed. Four hours thereafter, a supplementary addition of EX293 culture medium, 2 mM glutamine and 50 μg/L IGF-1 was performed and the culture was continued at 135 rpm. After a further 24 hours, 3.8 mM sodium valproate (VPA) was added. After 5-6 days culturing, the supernatant of the transient expression culture was collected and Protein A affinity chromatography was used to initially purify and obtain hPD-L1-Fc, hPD-L1-muFc and hPD-1-Fc protein samples for use in the following examples. Protein samples thus obtained were subject to preliminary testing using SDS-PAGE, and target bands were clearly visible.

### Example 2: Screening for Anti-hPD-L1 Antibodies in Yeast Display Library, Cloning Expression and Identification

Yeast display technology was used to screen for fully human antibodies for PD-L1. Cloning of VH and VL genes contained in spleen and lymph node IgM and IgG cDNA obtained from 21 healthy human subjects was performed to construct an scFV yeast display library (the connecting sequence between VH and VL was the connecting peptide

(SEQ ID NO: 67)
GGGGSGGGGSGGGGS

and the connecting peptide) storage capacity of the library was 5×10⁸. A 10× capacity yeast library was revived and yeasts were induced to express antibodies on their surface; 100 nM of biotinylated hPD-L1 antigen magnetic beads were used to perform two rounds of enrichment, after which a further two rounds of enrichment were performed using an anti-myc antibody and biotinylated hPD-L1 flow sorting. Yeasts thus obtained were plated and single clones were picked. Monoclonal yeasts which were subject to amplification and induction of expression were further subjected to a staining analysis using an anti-myc antibody as well as biotinylated hPD-L1 or the control antigen hPD-1 and yeasts which were antigen-positive or control-negative were assessed as being positive yeast.

FACS confirmed yeast clones were subject to yeast colony PCR and sequencing using the following PCR primers:

pNL6-F:
(SEQ ID NO: 78)
GTACGAGCTAAAAGTACAGTG;
pNL6-R:
(SEQ ID NO: 79)
TAGATACCCATACGACGTTC;

wherein the sequencing primer used was pNL6-R. The sequence results obtained after sequencing were subject to an alignment analysis using the BioEdit software package.

The single-chain antibody scFv gene obtained as described above and a previously obtained IgG1-Fc gene were fused and cloned into the commercial vector pEE6.4 (Lonza) using a double digest of Fermentas HindIII and EcoRI enzymes, after which cloning and plasmid miniprep were performed in accordance with standard molecular cloning procedures. Extracted plasmids were transiently expressed in HEK293 cells and purified using a protein A column.

hPD-L1-EGFP cells were resuspended in 0.5% PBS-BSA Buffer, after which the aforementioned purified anti-hPD-L1 scFv antibodies were added while at the same time, corresponding controls were established with 2 μg of a hIgG1 protein used as a negative control and hPD-1-Fc being added to the positive control. The secondary antibody used was anti-hIg-PE from eBioscience. Detection was performed via flow cytometry after staining was completed. The above method was used to identify antibodies capable of binding cell surface PD-L1 antigens.

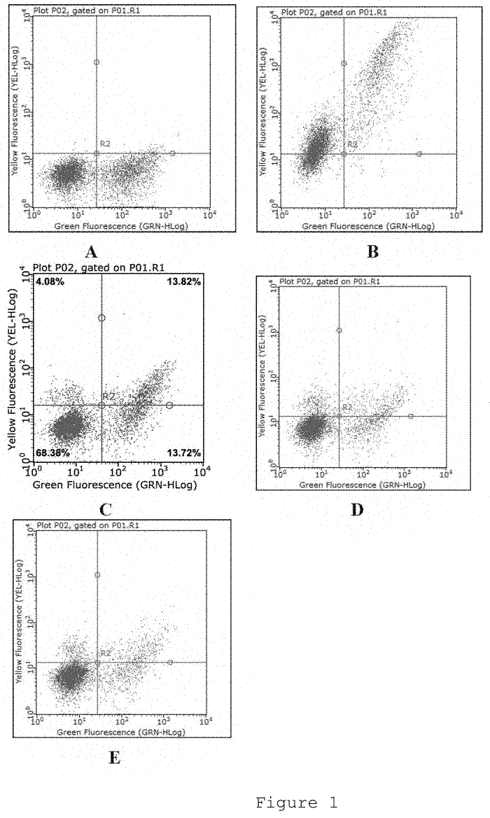

hPD-L1-EGFP cells were resuspended in 0.5% PBS-BSA Buffer, after which the aforementioned purified anti-hPD-L1 scFv antibodies were added while at the same time, a negative control was established with 2 μg of a hIgG1 protein used as a negative control; 0.3 μg of hPD-1-Fc-biotin was added to all samples and SA-PE from eBioscience was used as a secondary antibody; detection was performed via flow cytometry after staining was completed, and the results are shown in FIG. 1. The above method was used to identify antibodies capable of blocking cell surface PD-L1 antigens and PD-1 binding.

After screening and identification three antibody strains which showed favorable characteristics were obtained: B50, B60 and BII61. As can be seen in the results, all three anti-hPD-L1 antibody strains were able to block binding with the hPD-1 receptor.

The connecting peptide sequence

(SEQ ID NO: 67)
GGGGSGGGGSGGGGS

was contained between the heavy chain and light chain variable regions of the aforementioned antibody.

The amino acid sequence of the B50 heavy chain variable region was:

(SEQ ID NO: 51)
QVQLQQSGPGLVKPSQTLSLTCAISGDSVSSTKAAWYWIRQSPSRGLEWL
GRTYFRSKWYNDYADSVKSRLTINPDTSKNQFSLQLKSVSPEDTAVYYCA
RGQYTAFDIWGQGTMVTVSS;

wherein the underlined sections constitute CDR1, 2 and 3 and correspond to SEQ ID NO: 13-15 respectively and the non-underlined sections constitute FR1, 2, 3 and 4 and correspond to SEQ ID NO: 38-41 respectively;

the corresponding DNA sequence was:

(SEQ ID NO: 59)
CAGGTACAGCTGCAGCAGTCAGGTCCAGGACTGGTGAAGCCCTCGCAGAC
CCTCTCACTCACCTGTGCCATCTCCGGGGACAGTGTCTCTAGCACCAAGG
CTGCTTGGTACTGGATCAGGCAGTCCCTTCGAGAGGCCTTGAGTGGCTGG
GAAGGACATACTTCCGGTCCAAGTGGTATAATGACTATGCCGACTCTGTG
AAAAGTCGATTAACCATCAACCCAGACACATCCAAGAACCAGTTCTCCCT
GCAATTAAGTCTGTGAGTCCCGAGGACACGGCTGTGTATTACTGTGCAAG
AGGGCAATACACTGCTTTTGATATCTGGGGCCAAGGGACAATGGTCACCG
TCTCTTCA;

the amino acid sequence of the light chain variable region was:

(SEQ ID NO: 56)
QSALIQPASVSGSPGQSITISCTGTSSDVGGYDLVSWYQQYPGQAPRLII
YEVIKRPSGISDRFSGSKSGNTASLTISGLQAEDEADYYCSSYAGRRLHG
VFGGGTQLTVL;

wherein the underlined sections constitute CDR1, 2 and 3 and correspond to SEQ ID NO: 21, 17 and 18 respectively and the non-underlined sections constitute FR1, 2, 3 and 4 and correspond to SEQ ID NO: 42-45 respectively;

and the corresponding DNA sequence was:

(SEQ ID NO: 66)
CAGTCTGCTCTGATTCAGCCTGCCTCCGTGTCTGGGTCCCCTGGACAGTC
GATCACTATCTCCTGTACTGGCACCAGTAGTGATGTTGGAGGTTATGACC
TTGTCTCCTGGTACCAACAGTACCCGGCCAAGCCCCCAGACTCATCATTT
ATGAGGTCATTAAGCGGCCCTCAGGGATTTCTGATCGCTTCTCTGGTTCC
AAGTCTGGCAACACGGCCTCCCTGACAATCTCTGGGCTCCAGGCTGAGGA
CGAGCTGATTATTATTGCAGCTCATATGCAGGTAGACGTCTTCATGGTGT
GTTCGGAGGAGGCACCCAGCTGACCGTCCTC.

The amino acid sequence of the B60 heavy chain variable region was:

(SEQ ID NO: 53)
QVQLVQSGAEVKKPASSVKVSCTASGGSFSTYAISWVRQAPGQGLEWMGGI
IPIFGTTKYAQRFQGRVTITADESTTTAYMELSSLISDDTALYYCTTSRGF
SYGWFDYWGQGTLVTVSS;

wherein the underlined sections constitute CDR1, 2 and 3 and correspond to SEQ ID NO: 1, 2 and 19 respectively and the non-underlined sections constitute FR1, 2, 3 and 4 and correspond to SEQ ID NO: 22-25 respectively;

the corresponding DNA sequence was:

(SEQ ID NO: 60)
CAGGTCCAGCTTGTGCAGTCTGGAGCTGAGGTGAAGAAGCCTGCGTCCTCG
GTCAAAGTCTCCTGCACGGCTTCTGGCGGCTCCTTCAGCACCTATGCTATC
AGTTGGGTGCGACAGGCTCCTGGACAGGGCTTGAATGGATGGGCGGGATCA
TCCCCATCTTTGGTACAACTAAGTACGCACAGAGGTTCCAGGGCAGGGTCA
CGATTACCGCGGACGAATCGACGACCACAGCCTACATGGAGCTGAGCAGCT
GATATCTGACGACACGGCCCTGTATTATTGTACGACGTCTCGTGGATTCAG
CTATGGCTGGTTTGACTACTGGGGCCAGGGTACCCTGGTCACCGTCTCCTC
A;

the amino acid sequence of the light chain variable region was:

(SEQ ID NO: 48)
EIVMTQSPATLSLSPGERATLSCRASQSVGIHLAWYQQKLGQAPRLLIYGA
SSRATGIPDRFSGSGSGTDFTLTISRLEPEDFAVYYCQQYGSLPRTFGQGT
KVEIK;

wherein the underlined sections constitute CDR1, 2 and 3 and correspond to SEQ ID NO: 4-6 respectively and the non-underlined sections constitute FR1, 2, 3 and 4 and correspond to SEQ ID NO: 26-29 respectively;

and the corresponding DNA sequence was:

(SEQ ID NO: 62)
GAAATTGTAATGACACAGTCTCCAGCCACCCTGTCTTTGTCTCCAGGGGAA
AGAGCCACCCTCTCCTGTAGGGCCAGTCAGAGTGTTGGCATACACTTAGCC
TGGTACCAACAGAAACTTGGCCAGGCTCCCAGGCTCCTCATCTATGGTGCA
TCCAGTAGGGCCACTGGCATCCCAGACAGGTTCAGTGGCAGTGGGTCTGGG
ACAGATTCACTCTCACCATCAGCAGACTGGAGCCTGAAGATTTTGCAGTGT
ATTACTGTCAGCAGTATGGTTCTTTACCTCGGACGTTCGGCCAAGGGACCA
AGGTGGAAATCAAA.

The amino acid sequence of the BII61 heavy chain variable region was:

(SEQ ID NO: 54)
QVQLQQSGPGLVKPSQTLSLTCAISGDSVSSNSASWNWIRQSPSRGLEWLG
RTYYRSKWYDDYADVSVKSRISINPDTSKNQFSLQLNSVTPEDTAVYYCAR
SQGRYFVNYGMDVWGQGTTVTVSS;

wherein the underlined sections constitute CDR1, 20 and 3 and correspond to SEQ ID NO: 7, 2 and 9 respectively and the non-underlined sections constitute FR1, 2, 3 and 4 and correspond to SEQ ID NO: 30-33 respectively;

the corresponding DNA sequence was:

(SEQ ID NO: 61)
CAGGTACAGCTGCAGCAGTCAGGTCCAGGACTGGTGAAGCCCTCGCAGACC
CTCTCACTCACCTGTGCCATCTCCGGGGACAGTGTCTCTAGCAACAGTGCT
TCTTGGAACTGGATCAGGCAGTCCCATCGAGAGGCCTTGAGTGGCTGGGAA
GGACATATTACAGGTCCAAATGGTATGATGATTATGCAGTATCTGTGAAAA
GTCGAATCAGCATCAACCCAGACACATCCAAGAACCAGTTCTCCCTGCAGT
GAACTCTGTGACTCCCGAGGACACGGCTGTGTATTACTGTGCAAGAAGCCA
GGGACGATATTTTGTCAACTACGGTATGGACGTCTGGGGCCAAGGGACCAC
GGTCACCGTCTCCTCA;

the amino acid sequence of the light chain variable region was:

(SEQ ID NO: 55)
DIRLTQSPSSLSASVGDRITITCRASQSISSYLNWYQQKPGKAPKLLIY
GASSLQSGVPSRFSGSGSGTDFTLTISSLQPEDVATYYCQQSYFTPRGI
TFGPGTKVDIK;

wherein the underlined sections constitute CDR1, 2 and 3 and correspond to SEQ ID NO: 10-12 respectively and the non-underlined sections constitute FR1, 2, 3 and 4 and correspond to SEQ ID NO: 34, 46, 36 and 37 respectively;

and the corresponding DNA sequence was:

(SEQ ID NO: 65)
GACATCCGGTTGACCCAGTCTCCATCTTCCCTGTCTGCATCTGTAGGAG
ACAGAATCACCATCACTTGCCGGGCAAGTCAGAGCATTAGCAGTTATTT
AAATTGGTATCAACAGAAACCAGGGAAAGCCCTAAGCTCCTGATCTATG
GTGCATCCAGTTTGCAAAGTGGGGTCCCATCAAGGTTCAGTGGCAGTGG
ATCTGGGACAGATTTCACTCTCACCATCAGCAGTCTGCAACCTGAAGAT
GTTGCAACTACTACTGTCAACAGAGTTACTTTACCCCCCGCGGGATCAC
TTTCGGCCCTGGGACCAAAGTGGATATCAAA.

### Example 3: Construction of Anti-hPD-L1 scFv Improved Affinity Yeast Library

A standard PCR reaction was respectively performed using pEE6.4-B50-Fc, pEE6.4-B60-Fc and pEE6.4-BII61-Fc plasmids as templates, and

pEE6.4-F:
(SEQ ID NO: 80)
TCTGGTGGTGGTGGTTCTGCTAGC
and
cMyc-BBXhoI:
(SEQ ID NO: 81)
GCCAGATCTCGAGCTATTACAAGTCTTCTTCAGAAATAAGCTTTTGTTC
TAGAATTCCG

as primers. PCR products were purified and cloned into the commercial pCT302 vector commercial (addgene: #41845) using Fermentas NheI and BglII, to obtain the recombinant plasmids pCT302-B50, pCT302-B60 and pCT302-BII61. Next, error prone PCR was used based on the method detailed in Ginger et al. (2006) Nat Protoc 1(2):755-68 to obtain scFv randomly mutated PCR products. The primers used were

T7 proshort:
(SEQ ID NO: 82)
TAATACGACTCACTATAGGG
and
Splice 4/L:
(SEQ ID NO: 83)
GGCAGCCCCATAAACACACAGTAT.

The PCR products thus obtained were purified using a Fermentas GeneJET DNA Purification Kit and then concentrated via ethanol precipitation to a concentration greater than 1 μg/μl. Fermentas NheI and BamHI were used to perform a double digestion of the commercial vector pCT302 and at the same time, the Fermentas FastAP dephosphorylation enzyme was used to perform dephosphorylation of the vector, after which a Fermentas GeneJET DNA Purification Kit was again used to perform purification and ethanol precipitation was performed to concentrate the product to a concentration greater than 1 μg/μl. Yeast electro-transformation and in vivo recombination were performed in accordance with the method described in Ginger et al. (2006) Nat. Protoc. 1(2): 755-68 to obtain an affinity matured yeast library.

### Example 4: Screening for Yeast Expressing Anti-hPD-L1 scFv with Improved Affinity

The affinity matured yeast library obtained as described above was subjected to two rounds of flow sorting using 10 nM and 1 nM of a hPD-L1-Fc protein, and yeast products thus obtained were plated and monoclones were picked for identification. Low concentration antigen staining was used to perform flow staining to identify yeast monoclones which showed increased affinity, by using previously obtained wildtype yeast as a control.

Yeast clones which had passed FACS verification were subject to yeast colony PCR and sequencing using the methodology described above. The scFv gene obtained following affinity maturation and a previously obtained IgG1-Fc gene were fused and cloned into the commercial vector pEE6.4 using a double digest of Fermentas HindIII and EcoRI enzymes, after which cloning and plasmid miniprep were performed in accordance with standard molecular cloning procedures. Extracted plasmids were transiently expressed in HEK293 cells and purified using a protein A column.

The antibody binding capacity and blocking capacity were measured using the method described in Example 2.

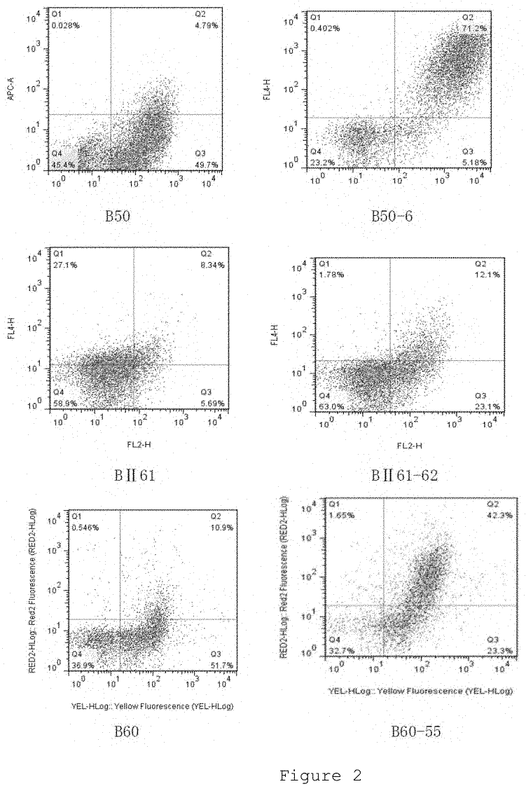

See FIG. 2 for the results of the binding capacity testing; the results show that the three antibody strains obtained following affinity maturation had significantly increased affinity.

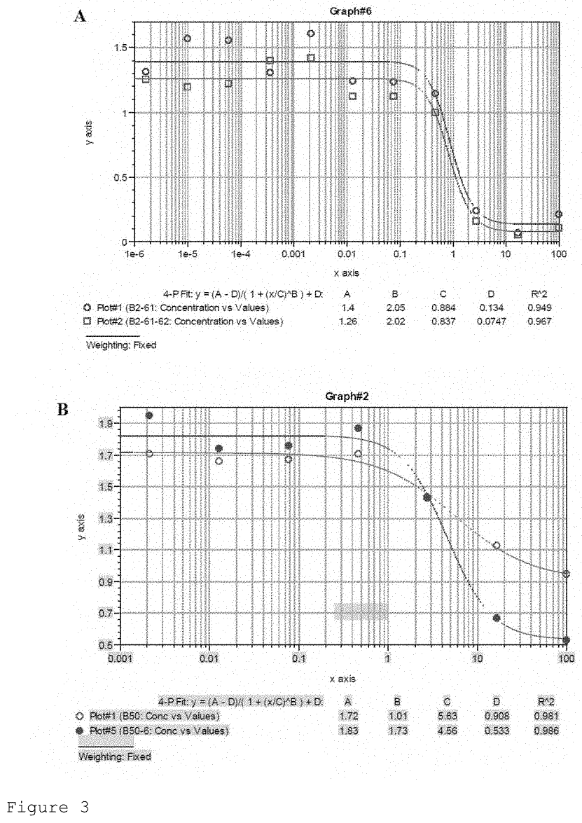

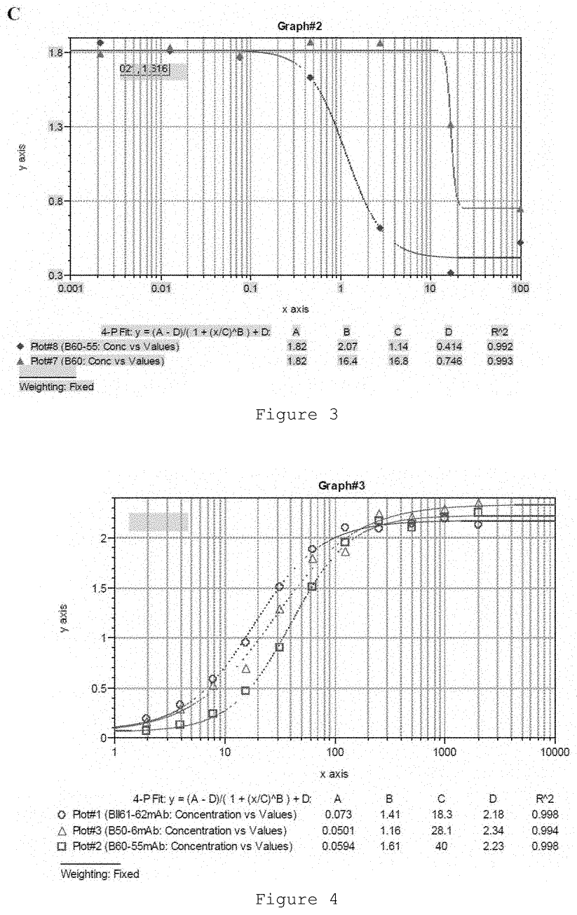

See FIG. 3 for the results of the blocking capacity testing; the results show that the IC50 values for competitive binding to PD-L1 in competition with PD-1 obtained for the three antibody strains obtained following affinity maturation were 0.837 μg/ml for BII61-62 (0.884 μg/ml for BII61), 4.56 μg/ml for B50-6 (5.63 μg/ml for B50) and 1.14 μg/ml for B60-55 (16.8 μg/ml for B60), respectively.

Following affinity maturation, three anti-hPD-L1 scFv antibody sequences showing increased affinity, i.e. B50-6, B60-55 and BII61-62, were obtained. Compared with B50, B50-6 showed an amino acid mutation from D to N in its VL CDR1; compared with B60, B60-55 showed an amino acid mutation from S to N in its VH CDR3; and compared with BII61, BII61-62 showed an amino acid mutation from S to G in its VH CDR2 as well as an amino acid mutation from I to V in its VL FR2. The connecting peptide sequence

(SEQ ID NO: 67)
GGGGSGGGGSGGGGS

was contained between the heavy chain and light chain variable regions of the aforementioned antibody.

The amino acid sequence of the B50-6 heavy chain variable region was:

(SEQ ID NO: 51)
QVQLQQSGPGLVKPSQTLSLTCAISGDSVSSTKAAWYWIRQSPSRGLEWL
GRTYFRSKWYNDYADSVKSRLTINPDTSKNQFSLQLKSVSPEDTAVYYCA
RGQYTAFDIWGQGTMVTVSS;

wherein the underlined sections constitute CDR1, 2 and 3 and correspond to SEQ ID NO: 13-15 respectively and the non-underlined sections constitute FR1, 2, 3 and 4 and correspond to SEQ ID NO: 38-41 respectively;

the corresponding DNA sequence was:

(SEQ ID NO: 59)
CAGGTACAGCTGCAGCAGTCAGGTCCAGGACTGGTGAAGCCCTCGCAGAC
CCTCTCACTCACCTGTGCCATCTCCGGGGACAGTGTCTCTAGCACCAAGG
CTGCTTGGTACTGGATCAGGCAGTCCCTTCGAGAGGCCTTGAGTGGCTGG
GAAGGACATACTTCCGGTCCAAGTGGTATAATGACTATGCCGACTCTGTG
AAAAGTCGATTAACCATCAACCCAGACACATCCAAGAACCAGTTCTCCCT
GCAATTAAGTCTGTGAGTCCCGAGGACACGGCTGTGTATTACTGTGCAAG
AGGGCAATACACTGCTTTTGATATCTGGGGCCAAGGGACAATGGTCACCG
TCTCTTCA;

the amino acid sequence of the light chain variable region was:

(SEQ ID NO: 52)
QSALIQPASVSGSPGQSITISCTGTSSNVGGYDLVSWYQQYPGQAPRLII
YEVIKRPSGISDRFSGSKSGNTASLTISGLQAEDEADYYCSSYAGRRLHG
VFGGGTQLTVL;

wherein the underlined sections constitute CDR1, 2 and 3 and correspond to SEQ ID NO: 16-18 respectively and the non-underlined sections constitute FR1, 2, 3 and 4 and correspond to SEQ ID NO: 42-45 respectively;

the corresponding DNA sequence was:

(SEQ ID NO: 64)
CAGTCTGCTCTGATTCAGCCTGCCTCCGTGTCTGGGTCCCCTGGACAGTC
GATCACTATCTCCTGTACTGGCACCAGTAGTAATGTTGGAGGTTATGACC
TTGTCTCCTGGTACCAACAGTACCCGGGCCAAGCCCCCAGACTCATCATT
TATGAGGTCATTAAGCGGCCCTCAGGGATTTCTGATCGCTTCTCTGGTTC
CAAGTCTGGCACACGGCCTCCCTGACAATCTCTGGGCTCCAGGCTGAGGA
CGAGGCTGATTATTATTGCAGCTCATATGCAGGTAGACGTCTTCATGGTG
TGTTCGGAGGAGGCACCCAGCTGACCGTCCTC;

the amino acid sequence of the B60-55 heavy chain variable region was:

(SEQ ID NO: 47)
QVQLVQSGAEVKKPASSVKVSCTASGGSFSTYAISWVRQAPGQGLEWMGGI
IPIFGTTKYAQRFQGRVTITADESTTTAYMELSSLISDDTALYYCTTSRGF
NYGWFDYWGQGTLVTVSS;

wherein the underlined sections constitute CDR1, 2 and 3 and correspond to SEQ ID NO: 1-3 respectively and the non-underlined sections constitute FR1, 2, 3 and 4 and correspond to SEQ ID NO: 22-25 respectively;

the corresponding DNA sequence was:

(SEQ ID NO: 57)
CAGGTCCAGCTTGTGCAGTCTGGAGCTGAGGTGAAGAAGCCTGCGTCCTCG
GTCAAAGTCTCCTGCACGGCTTCTGGCGGCTCCTTCAGCACCTATGCTATC
AGTTGGGTGCGACAGGCTCCTGGACAGGGCTTGAATGGATGGGCGGGATCA
TCCCCATCTTTGGTACAACTAAGTACGCACAGAGGTTCCAGGGCAGGGTCA
CGATTACCGCGGACGAATCGACGACCACAGCCTACATGGAGCTGAGCAGCT
GATATCTGACGACACGGCCCTGTATTATTGTACGACGTCTCGTGGATTCAA
CTATGGCTGGTTTGACTACTGGGGCCAGGGTACCCTGGTCACCGTCTCCTC
A;

the amino acid sequence of the light chain variable region was:

(SEQ ID NO: 48)
EIVMTQSPATLSLSPGERATLSCRASQSVGIHLAWYQQKLGQAPRLLIYGA
SSRATGIPDRFSGSGSGTDFTLTISRLEPEDFAVYYCQQYGSLPRTFGQGT
KVEIK;

wherein the underlined sections constitute CDR1, 2 and 3 and correspond to SEQ ID NO: 4-6 respectively and the non-underlined sections constitute FR1, 2, 3 and 4 and correspond to SEQ ID NO: 26-29 respectively;

the corresponding DNA sequence was:

(SEQ ID NO: 62)
GAAATTGTAATGACACAGTCTCCAGCCACCCTGTCTTTGTCTCCAGGGGAA
AGAGCCACCCTCTCCTGTAGGGCCAGTCAGAGTGTTGGCATACACTTAGCC
TGGTACCAACAGAAACTTGGCCAGGTCCCAGGCTCCTCATCTATGGTGCAT
CCAGTAGGGCCACTGGCATCCCAGACAGGTTCAGTGGCAGTGGGTCTGGGA
CAGACTTCACTCTCACCATCAGCAGACTGGAGCCTGAAGATTTTGCAGTGA
TTACTGTCAGCAGTATGGTTCTTTACCTCGGACGTTCGGCCAAGGGACCAA
GGTGGAAATCAAA;

the amino acid sequence of the BII61-62 heavy chain variable region was:

(SEQ ID NO: 49)
QVQLQQSGPGLVKPSQTLSLTCAISGDSVSSNSASWNWIRQSPSRGLEWLG
RTYYRSKWYDDYAVSVKGRISINPDTSKNQFSLQLNSVTPEDTAVYYCARS
QGRYFVNYGMDVWGQGTTVTVSS;

wherein the underlined sections constitute CDR1, 2 and 3 and correspond to SEQ ID NO: 7-9 respectively and the non-underlined sections constitute FR1, 2, 3 and 4 and correspond to SEQ ID NO: 30-33 respectively;

the corresponding DNA sequence was:

(SEQ ID NO: 58)
CAGGTACAGCTGCAGCAGTCAGGTCCAGGACTGGTGAAGCCCTCGCAGACC
CTCTCACTCACCTGTGCCATCTCCGGGGACAGTGTCTCTAGCAACAGTGCT
TCTTGGAACTGGATCAGGCAGTCCCATCGAGAGGCCTTGAGTGGCTGGGAA
GGACATATTACAGGTCCAAATGGTATGATGATTATGCAGTATCTGTGAAAG
GTCGAATCAGCATCAACCCAGACACATCCAAGAACCAGTTCTCCCTGCAGT
GAACTCTGTGACTCCCGAGGACACGGCTGTGTATTACTGTGCAAGAAGCCA
GGGACGATATTTTGTCAACTACGGTATGGACGTCTGGGGCCAAGGGACCAC
GGTCACCGTCTCCTCA;

the amino acid sequence of the light chain variable region was:

(SEQ ID NO: 50)
DIRLTQSPSSLSASVGDRITITCRASQSISSYLNWYQQKPGKAPKLLVYG
ASSLQSGVPSRFSGSGSGTDFTLTISSLQPEDVATYYCQQSYFTPRGITF
GPGTKVDIK;

wherein the underlined sections constitute CDR1, 2 and 3 and correspond to SEQ ID NO: 10-12 respectively and the non-underlined sections constitute FR1, 2, 3 and 4 and correspond to SEQ ID NO: 34-37 respectively;

and the corresponding DNA sequence was:

(SEQ ID NO: 63)
GACATCCGGTTGACCCAGTCTCCATCTTCCCTGTCTGCATCTGTAGGAGA
CAGAATCACCATCACTTGCCGGGCAAGTCAGAGCATTAGCAGTTATTTAA
ATTGGTATCAACAGAAACCAGGGAAAGCCCTAAGCTCCTGGTCTATGGTG
CATCCAGTTTGCAAAGTGGGGTCCCATCAAGGTTCAGTGGCAGTGGATCT
GGGACAGATTTCACTCTCACCATCAGCAGTCTGCAACCTGAAGATGTTGC
AACTACTACTGTCAACAGAGTTACTTTACCCCCCGCGGGATCACTTTCGG
CCCTGGGACCAAAGTGGATATCAAA.

### Example 5: Formatting of scFv Antibody to IgG Antibody

A human IgG1 constant region amino acid sequence was obtained based on the amino acid sequence of the constant region of human immunoglobulin gamma1 (IgG1) contained in the Uniprot protein database (P01857). The online tool DNAworks (http://helixweb.nih.gov/dnaworks/) was used to design corresponding encoding DNA sequences to obtain a human IgG1 constant region gene and the VH sequences of the heavy chain variable regions of B50-6, B60-55 and BII61-61 obtained via screening were spliced together with the human IgG1 constant region gene while at the same time, the following signal peptide sequence was added to the 5′ end of the VH:

(SEQ ID NO: 84)
ATGGAGACAGACACACTCCTGCTATGGGTACTGCTGCTCTGGGTTCCAGG
TTCCACCGGT;

the spliced gene was synthesized and double digestion was performed using Fermentas HindIII and EcoRI enzymes to clone the gene into the vector pEE6.4 to obtain pEE6.4-B50-6HC; pEE6.4-B60-55HC; and pEE6.4-BII61-62HC. A human Kappa light chain constant region amino acid sequence was obtained based on the amino acid sequence of the constant region of human immunoglobulin Kappa contained in the Uniprot protein database (P01834). The online tool DNAworks (http://helixweb.nih.gov/dnaworks/) was used to design corresponding encoding DNA sequences to obtain a human Kappa light chain constant region gene and the VL sequences of the heavy chain variable regions of B60-55 and BII61-61 obtained via screening were spliced together with the human Kappa light chain constant region gene while at the same time, the following signal peptide sequence was added to the 5′ end of the VL:

(SEQ ID NO: 84)
ATGGAGACAGACACACTCCTGCTATGGGTACTGCTGCTCTGGGTTCCAGG
TTCCACCGGT;

the online tool DNAworks (http://helixweb.nih.gov/dnaworks/) was used to design corresponding encoding DNA sequences to obtain a human lambda (λ) light chain constant region gene and the VL sequence of the light chain variable region of B50-6 obtained via screening were spliced together with the human lambda light chain constant region gene while at the same time the following signal peptide sequence was added to the 5′ end of the VL:

(SEQ ID NO: 84)
ATGGAGACAGACACACTCCTGCTATGGGTACTGCTGCTCTGGGTTCCAGG
TTCCACCGGT;

and the spliced gene was synthesized and double digestion was performed using Fermentas HindIII and EcoRI enzymes to clone the gene into the vector pEE12.4 (Lonza) to obtain pEE12.4-B50-6LC; pEE12.4-B60-55LC; and pEE12.4-BII61-62LC.

Heavy chain and light chain plasmids obtained as described above were prepared using an AidLab Maxiprep Kit (PL14). Recombinantly constructed light and heavy chain plasmids were co-transfected into HEK293 cells to express the antibody. The recombinant expression plasmid was diluted with a Freestyle293 culture medium and added to a PEI (polyethylenimine) solution required for transformation, after which each plasmid/PEI mixture was each separately added to a cell suspension and left to culture at 37° C., 10% CO₂ and 90 rpm; at the same time, a supplementary addition of 50 μg/IGF-1 was performed. Four hours thereafter, a supplementary addition of EX293 culture medium, 2 mM glutamine and 50 μg/L IGF-1 was performed and the culture was continued at 135 rpm. After a further 24 hours, 3.8 mM VPA was added. After 5-6 days culturing, the supernatant of the transient expression culture was collected and Protein A affinity chromatography was used to purify and obtain anti-hPD-L1 B50-6, B60-55 and BII61-62 mAb antibodies.

the IgG1 chain constant region amino acid sequence was:

(SEQ ID NO: 68)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIKTISKAKGQPREPQVYTLPPSRDELTKNQVSLTCL
VKGFYPSDIAVEWESNGQPENNYKTTPPVLDSDGSFELYSKLTVDKSRWQ
QGNVESCSVMHEALHNHYTQKSLSLSPGK;

the IgG1 chain constant region nucleic acid sequence was:

(SEQ ID NO: 69)
GCCAGCACTAAGGGGCCCTCTGTGTTTCCACTCGCCCCTTCTAGCAAAAG
CACTTCCGGAGGCACTGCAGCACTCGGGTGTCTGGTCAAAGATTATTTCC
CTGAGCCAGTCACCGTGAGCTGGAACTTGGCGCCCTCACCTCCGGGGTTC
ACACCTTTCCAGCCGTCCTGCAGTCCTCCGGCCTGTACTCCCTGAGCAGC
GTCGTTACCGTGCCATCCTCTTCTCTGGGGACCCAGACATACATCTGCAA
TGTCACCATAAGCCTAGCAACACCAAGGTGGACAAAAAGGTCGAGCCAAA
GAGCTGCGATAAGACACACACCTGCCCTCCATGCCCCGCACCTGAACTCC
TGGGCGGGCCTTCCGTTTTCCTGTTTCCTCCAAGCCCAAGGATACACTGA
TGATTAGCCGCACCCCCGAAGTCACTTGCGTGGTGGTGGATGTGAGCCAT
GAAGATCCAGAAGTTAAGTTTAACTGGTATGTGGACGGGGTCGAGGTGCA
CAATGCTAAACAAAGCCCAGGGAGGAGCAATATAACTCCACATACAGAGT
GGTGTCCGTTCTGACAGTCCTGCACCAGGACTGGCTGAACGGGAAGGAAT
ACAAGTGCAAGGTGTCTAATAAGGCACTGCCAGCCCCATAGAGAAGACAA
TCTCTAAAGCTAAAGGCCAACCACGCGAGCCTCAGGTCTACACACTGCCA
CCATCCAGGGACGAACTGACCAAGAATCAGGTGAGCCTGACTTGTCTCGT
CAAAGGATTCTACCAAGCGACATCGCCGTGGAGTGGGAATCCAACGGCCA
ACCAGAGAACAACTACAAGACCACCCCACCAGTCCTGGACTCTGATGGGA
GCTTTTTCCTGTATTCCAAGCTGACAGTGGACAAGTCTCGTGGCAACAGG
GCAACGTGTTCAGCTGCTCCGTGATGCATGAAGCCCTGCATAACCACTAT
ACCCAGAAAAGCCTCAGCCTGTCCCCCGGGAAATAATGA;

the Kappa chain constant region amino acid sequence was:

(SEQ ID NO: 70)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC;

the Kappa chain constant region nucleic acid sequence was:

(SEQ ID NO: 71)
CGTACGGTGGCTGCACCATCTGTCTTCATCTTCCCGCCATCTGATGAGCA
GTTGAAATCTGGTACCGCTAGCGTTGTGTGCCTGCTGAATAACTTTTATC
CACGGGAGGCTAAGGTGCAGTGGAAAGGGACAATGCCCTCCAGAGCGGAA
ATAGCCAAGAGTCCGTTACCGAACAGGACTCTAAAGACTCTACATACTCC
CTGTCCTCCACACTGACCCTCTCCAAGGCCGACTATGAGAAACACAAGGT
TTACCATGCGAGGTCACACACCAGGGACTCTCCTCTCCCGTGACCAAGAG
CTTCAACCGGGGAGAATGC;

the B50-6 light chain (lambda) constant region amino acid sequence was:

(SEQ ID NO: 72)
GQPKAAPSVTLFPPSSEELQANKATLVCLVSDFYPGAVTVAWKADGSPVK
VGVETTKPSKQSNNKYAASSYLSLTPEQWKSHRSYSCRVTHEGSTVEKTV
APAECS;

and the B50-6 light chain (lambda) constant region nucleic acid sequence was:

(SEQ ID NO: 73)
GGTCAGCCCAAGGCTGCCCCCTCGGTCACTCTGTTCCCACCCTCCTCTGA
GGAGCTTCAAGCCAACAAGGCCACACTGGTGTGTCTCGTAAGTGACTTCT
ACCCGGGAGCCGTGACAGTGGCCTGGAGGCAGATGGCAGCCCCGTCAAGG
TGGGAGTGGAGACCACCAAACCCTCCAAACAAAGCAACAACAAGTATGCG
GCCAGCAGCTACCTGAGCCTGACGCCCGAGCAGTGGAAGTCCCACAGAAG
CTACGCTGCCGGGTCACGCATGAAGGGAGCACCGTGGAGAAGACAGTGGC
CCCTGCAGAATGCTCT.

### Example 6: Verification of Anti-hPD-L1 mAb Properties

Measurement of Purified Anti-hPD-L1 Antibody and hPD-L1 Binding Capacity (ELISA Method):

A coating buffer (50 mM carbonate-bicarbonate buffer, pH 9.6) was used to dilute hPD-L1-muFc to 2 μg/ml after which the solution was aliquoted at 100 μL/well and left to stand at 4° C. overnight. Liquid on the plate was then thrown off and washing was performed using PBST (pH 7.4, 0.05% Tween-20, V/V) and the sample was sealed in 3% BSA-PBS for 1 hour. The antibodies B50-6mAb, B60-55mAb and BII61-62mAb were each subject to twofold serial dilution starting from 2,000 ng/ml, for a total of 11 different concentrations with diluent (1% BSA-PBS) used as a control, and incubation was performed for 2 hours at 37° C. Goat anti-human IgG-HRP (goat anti-human IgG-HRP conjugated) was then added and incubation was performed for 1 hour. Soluble single-component TMB chromogenic substrate solution was then added and each sample was developed at room temperature in a dark environment for 5-10 minutes. 2 N H₂SO₄ was added at 50 μL/well to terminate the development reaction. Each sample was then placed on an MD SpectraMax Plus384 microplate reader and OD450 nm-650 nm values were read, after which the SoftMax Pro v5.4 software package was used to perform data processing and mapping analysis; the results are shown in FIG. 4.

Using the above method, the antigen-binding EC50 values of the three antibody strains were determined to be 40 μg/ml (B60-55 mAb), 18.3 μg/ml (BII61-62 mAb), and 28.1 μg/ml (B50-6 mAb).

Measurement of Purified Anti-hPD-L1 Antibody and hPD-L1 Binding Kinetics (SPR):

Measurements of the binding kinetics of the anti-PD-L1 antibodies B50-6 mAb, BII61-62 mAb and B60-55 mAb with respect to recombinant human PD-L1 were measured using surface plasmon resonance (SRP) conducted using the Biacore X100. Recombinant hPD-L1-Fc was directly coated onto a CM5 biosensor chip in order to obtain approximately 1000 response units (RU). For kinetics measurements, the antibody was diluted via a threefold serial dilution in HBS-EP+1×buffer (GE, Cat #: BR-1006-69) (from 1.37 nm to 1000 nm), sampling was performed at 25° C. for 120 seconds, with a dissociation time of 30 minutes, and regeneration was performed with 10 mM glycine-HCl (pH 2.0) for 120 seconds. A simple one-to-one Languir binding model (Biacore Evaluation Software Version 3.2) was used to calculate the association rate (kon) and dissociation rate (koff). The equilibrium dissociation constants (kD) was computed as the ratio of koff/kon.

See Table 1 for the measured anti-PD-L1 binding affinity values.

TABLE 1
Measurement of anti-hPD-L1 antibody
and hPD-L1 binding kinetics

| Designation | Kon (1/Ms) | Koff (1/s) | KD (M) |
|---|---|---|---|
| B50-6mAb | 1.672E+5 | 1.370E−2 | 8.193E−8 |
| B60-55mAb | 1.295E+6 | 2.222E−4 | 1.716E−10 |
| BII 61-62mAb | 9.795E+4 | 4.264E−4 | 4.353E−9 |

Measurement of Purified Anti-hPD-L1 Antibody Binding Capacity for hPD-L1 in Competition with hPD-1:

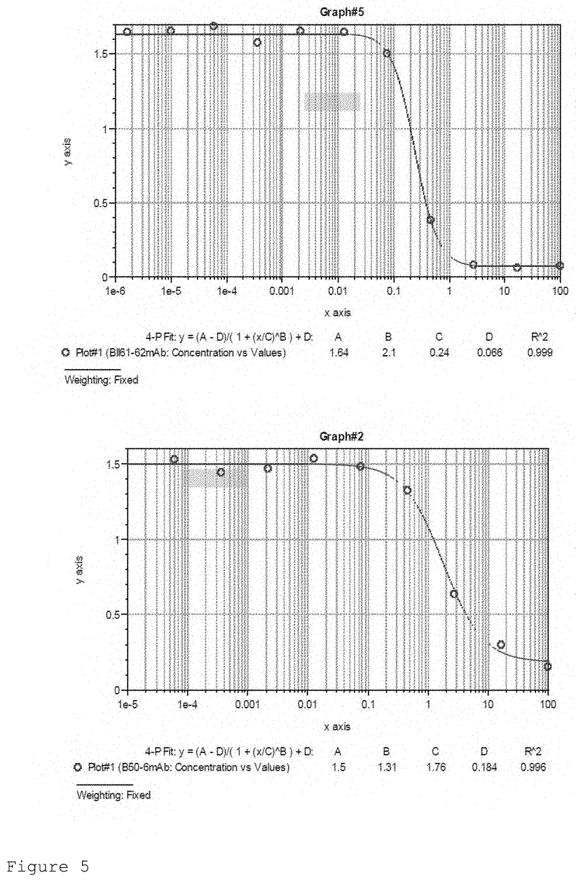

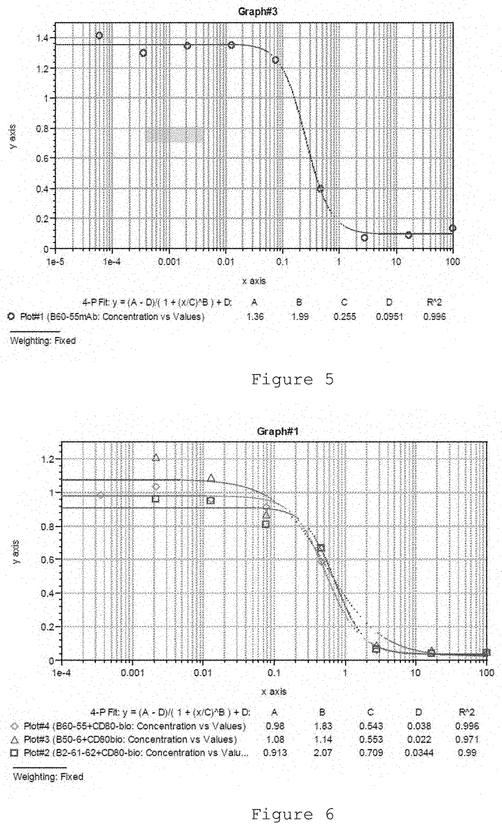

A coating buffer (50 mM carbonate-bicarbonate buffer, pH 9.6) was used to dilute hPD-L1-hIgG to 5 μg/ml after which the solution was left to stand at 4° C. overnight. Washing was performed using PBST (pH 7.4, 0.05% Tween-20, V/V) and the sample was sealed in 3% BSA-PBS for 1 hour. The concentration of anti-hPD-L1 mAb awaiting measurement was diluted to 100 μg/ml, after which a 1:6 serial dilution was performed using 1% BSA-PBST-0.05% Tween-20 (containing 10 μg/ml of hPD-1-hIgG-biotin) for a total of 9 different dilutions, and the dilutions were left to stand for 2 hours at 37° C. After the plate was washed, horseradish peroxidase-conjugated streptavidin (SA-HRP) was added and the sample was allowed to incubate at room temperature for 1.5 hours. Soluble single-component TMB chromogenic substrate solution was then added and each sample was developed at room temperature in a dark environment for 5-10 minutes, after which 2 N H₂SO₄ was added to terminate the development reaction. Each sample was then placed on an MD SpectraMax Plus384 microplate reader and OD450 nm-650 nm values were read, after which the SoftMax Pro v5.4 software package was used to perform data processing and mapping analysis; and the antibody competitiveness was analyzed based on measured data and IC50 values and the results are shown in FIG. 5.

Using the above method, the competitive antigen-binding IC50 values for PD-L1 of the three antibody strains with respect to PD-1 were determined to be 0.255 μg/ml 1.7 nM (B60-55), 0.24 μg/ml 1.6 nM (BII61-62), and 1.76 μg/ml 11.7 nM (B50-6).

Measurement of Purified Anti-hPD-L1 Antibody Binding Capacity for hPD-L1 in Competition with CD80:

Three antibody strain obtained via screening, B60-55, BII61-62 and B50-6, were evaluated to determine whether or not they were capable of blocking PD-L1 and CD80 binding via a competitive ELISA method. The specific method used was as follows: a coating buffer (50 mM carbonate-bicarbonate buffer, pH 9.6) was used to dilute hPD-L1-hFc to 5 μg/ml after which the solution was left to stand at 4° C. overnight. Washing was performed using PBST (pH 7.4, 0.05% Tween-20, V/V) and the sample was sealed in 3% BSA-PBS for 1 hour. The concentration of anti-hPD-L1 mAb awaiting measurement was diluted to 100 μg/ml, after which a 1:6 serial dilution was performed using 1% BSA-PBST-0.05% Tween-20 (containing 100 μg/ml of hCD80-hFc-biotin, R&D: 140-B1-100) for a total of 9 different dilutions, and the dilutions were left to stand for 2 hours at 37° C. After the plate was washed, horseradish peroxidase-labeled streptavidin-biotin (SA-HRP conjugated) was added and the sample was allowed to incubate at room temperature for 1.5 hours. Soluble single-component TMB chromogenic substrate solution was then added and each sample was developed at room temperature in a dark environment for 5-10 minutes, after which 2 N H₂SO₄ was added to terminate the development reaction. Each sample was then placed on an MD SpectraMax Plus384 microplate reader and OD450 nm-650 nm values were read, after which the SoftMax Pro v5.4 software package was used to perform data processing and mapping analysis; and the antibody competitiveness was analyzed based on measured data and IC50 values and the results are shown in FIG. 6.

Using the above method, the competitive antigen-binding IC50 values for PD-L1 of the three antibody strains with respect to CD80 were determined to be 0.543 μg/ml (B60-55), 0.709 μg/ml (BII61-62), and 0.553 μg/ml 11.7 nM (B50-6).

Verification to Determine Whether or not PD-L1 is Specifically Recognized: Binding of Purified Anti-hPD-L1 with hPD-L1, hPD-L2 and hB7H3

HEK293 cells containing hPD-L1-EGFP, hB7H3-EGFP and hPD-L2-EGFP which were constructed in Example 1 were suspended in a 0.5% PBS-BSA buffer, after which anti-hPD-L1 mAb protein was added (with hIgG Fc used as a negative control) and incubation over ice was performed for 20 minutes. After washing, the eBioscience secondary antibody anti-hIg-PE was added and the samples were left to stand on ice for 20 minutes. After washing, cells were resuspended in 500 μl of a 0.5% PBS-BSA Buffer and subject to measurement in a flow cytometer.

The results are shown in FIG. 6. As shown in the results, the three antibody strains were all able to bind with hPD-L1-EGFP cells but were unable to bind with hB7H3-EGFP and hPD-L2-EGFP cells, demonstrating good specificity.

Binding of Purified Anti-hPD-L1 with Murine PD-L1 (mPD-L1):

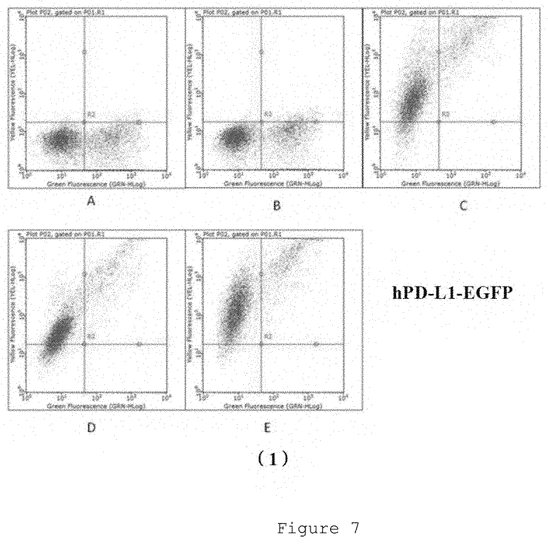

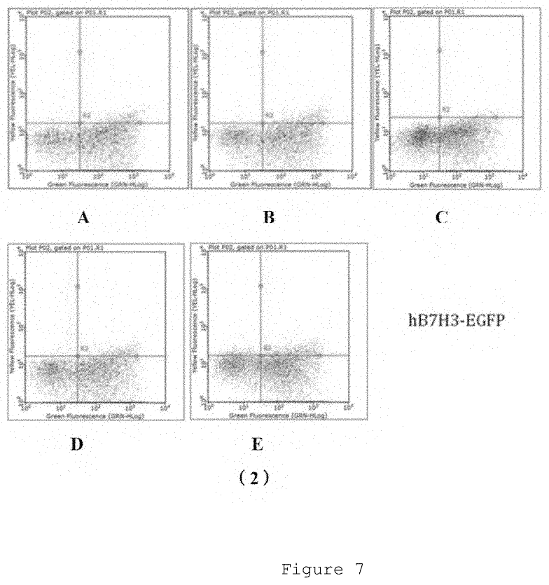

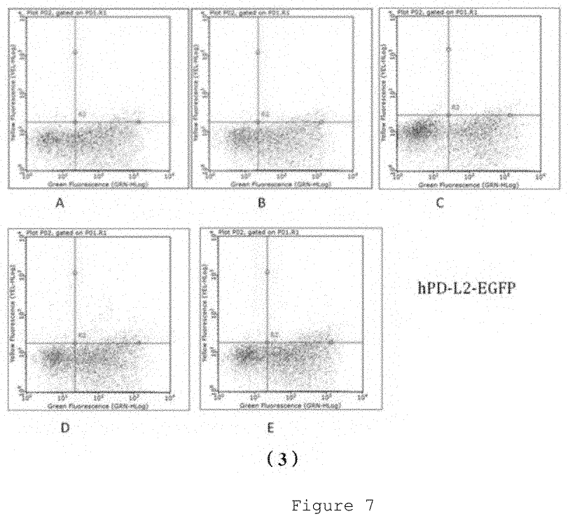

HEK293 cells containing hPD-L1-EGFP and mPD-L1-EGFP which were constructed in Example 1 were suspended in a 0.5% PBS-BSA buffer, after which target anti-hPD-L1 mAb was added (with hIgG Fc used as a negative control) and incubation over ice was performed for 20 minutes; washing was then performed, the eBioscience secondary antibody anti-hIg-PE was added and the samples were left to stand on ice for 20 minutes. After washing, cells were resuspended in a 0.5% PBS-BSA Buffer and subject to measurement in a flow cytometer. The results are shown in FIG. 7. As shown in the results, B50-6 mAb was capable of binding with murine PD-L1 (mPD-L1), while B60-55 and BII61-62 were not able to bind with mPD-L1.

Binding of Purified Anti-hPD-L1 with Cynomolgus Monkey PD-L1:

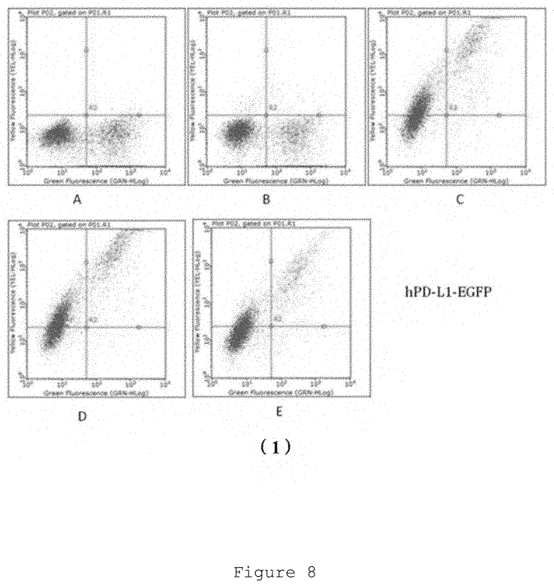

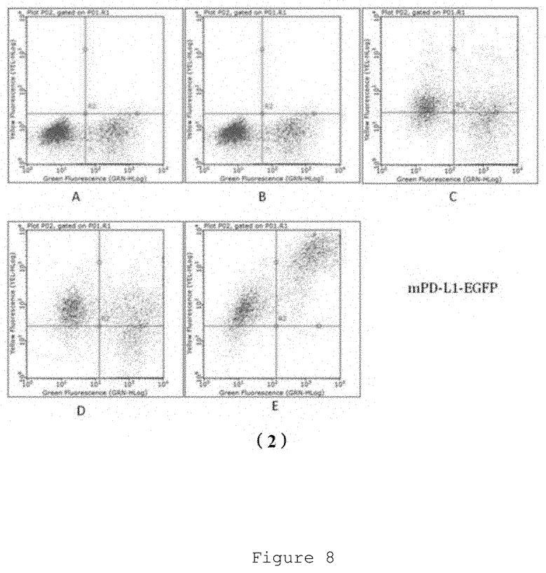

Cynomolgus monkey PBMCs were separated using a human lymphocyte separation medium (Tianjin Hao Yang) and cells were resuspended in RPMI complete medium, after which cell density was adjusted to 1 million cells/ml; subsequently, 2 million cynomolgus monkey PBMCs were added to a 24-well plate while phytohaemagglutinin (PHA) was simultaneously added to a final concentration of 2 μg/ml; cells were stimulated for 48 hours, after which they were collected, washed in a FACS buffer and subject to antibody staining. Isotype ctrl (anti-KLH) was used as a negative control and commercial PE-labeled anti-human PD-L1 antibodies (Biolegend: 329705) were used as a positive control. Using our in-house antibodies as primary antibodies, antibody staining was performed using anti-hIg-PE as a secondary antibody after washing was performed. Each staining step was followed by incubation at 4° C. for thirty minutes, and after staining was performed, a FACS buffer was used to wash cells twice via centrifugation, after which secondary antibodies were added or cells were fixed directly in 2% paraformaldehyde followed by an analysis using Guava. The results are shown in FIG. 8. The results showed that cynomolgus monkey T cells expressed PD-L1 after being stimulated with PHA and the three antibody strains which were produced were capable of binding with activated cynomolgus monkey T cells.

### Example 7: Measurement of PD-L1 Antibody Activation of CD4⁺ T Cells in a Dendritic Cell-T Cell Mixed Lymphocyte Reaction

Human lymphocyte separation medium (Tianjin Hao Yang) was used to separate out peripheral blood mononuclear cells (PBMCs) from enriched peripheral white blood cells obtained from healthy donors via density gradient centrifugation. Next, said cells were resuspended in serum-free RPMI1640 and cultured in a 10 cm dish for 1-2 hours, after which non-adherent cells were removed and cells were cultured in RPMI containing 10% FBS. Cytokines were added at final concentrations of 250 ng/ml for GM-CSF (Shanghai Primegene: 102-03) and 100 ng/ml for IL-4 (Shanghai Primegene: 101-04) and a fresh cytokine-containing medium was thereafter added every 2-3 days. On day 6 of the culture, 50 ng/ml TNF-alpha (Shanghai Primegene: 103-01) was used to induce cell maturation and cells were incubated for a further 24 hours. Mature dendritic cells were harvested and stained with HLA-DR antibody to verify maturation. Cells were then resuspended in a RPMI complete medium at a concentration of 200,000 cells/ml. 50 μl of the resulting suspension was added to each well of a 96-well U-bottom plate (Costar: 3799) and the cells were left to culture in an incubator.

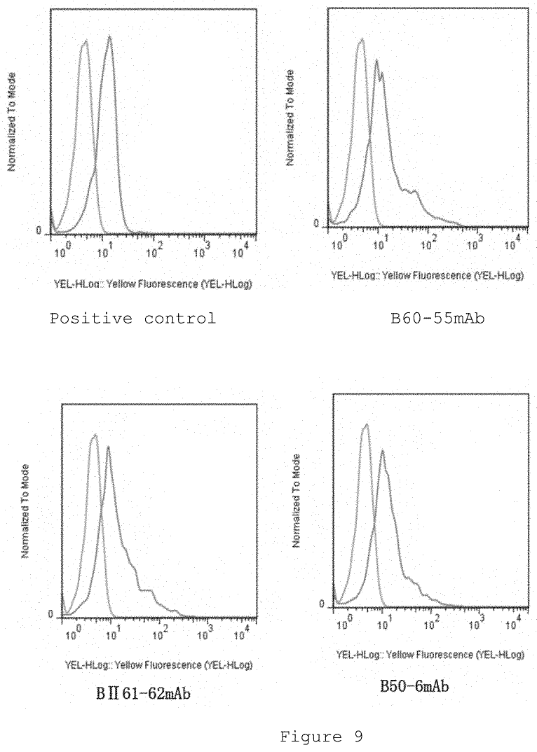

A magnetic bead isolation kit (Miltenyi Biotec: 130-096-533) was used to isolate CD4⁺T cells from PBMCs obtained from another donor according to the instructions provided. Cells were counted and resuspended in RPMI complete medium at a concentration of 2 million cells/ml, after which they were added to the 96-well U-bottom plate containing dendritic cells, with 50 μl being added to each well. 100 μl of PD-L1 antibodies which had been serially diluted in RPMI complete medium were added to each well to obtain final antibody concentrations of 100, 10, 1, 0.1, 0.01, 0.001 and 0 μg/ml. Cells were then cultured for five days, the supernatant was taken, and an IFN-γ ELISA detection kit (eBioscience) was used to detect IFN-γ levels in the supernatant. The results are shown in FIG. 9. The results show that PD-L1 antibodies can enhance CD4⁺ T cell secretion of γ-IFN in a mixed lymphocyte reaction; that is to say, PD-L1 antibodies enhanced T cell activation. The EC50 value obtained for BII61-62 was 0.078 μg/ml (equivalent to 0.5 nM) and the EC50 value obtained for B60-55 was 0.189 μg/ml (equivalent to 1.2 nM).

### Example 8: Inhibitory Activity of Anti-hPD-L1 Antibodies on Tumor Growth

It is already clear that many tumors express PD-1 ligands as a way of weakening the body's anti-tumor T cell responses. Characteristic increased expression levels of PD-L1 was been discovered in tumors and tumor infiltrating leukocytes in many different subjects, and said increased PD-L1 expression is often associated with poor prognosis. Murine tumor models have shown similar increases in PD-L1 expression in tumors and have also demonstrated the role of the PD-1/PD-L1 pathway in inhibiting tumor immunity.

Here we have provided experimental results which show that blocking PD-L1 affects tumor growth for MC38 cells (murine colorectal cancer cells) found in syngeneic C57B6 mice.

At Day 0, 1 million MC38 cells (generously provided by Professor Yangxin Fu of the University of Chicago) were inoculated subcutaneously in C57B6 mice; mice were then subject to a 10 mg/kg anti-PD-L1 (B50-6) or PBS intraperitoneal injection on days 0, 3, 7 and 10. Tumor dimensions were measured on day 3 and the tumor volume was computed to draw a tumor growth curve (see FIG. 10); the results show that anti-PD-L1 (B50-6) is capable of significantly inhibiting tumor growth.

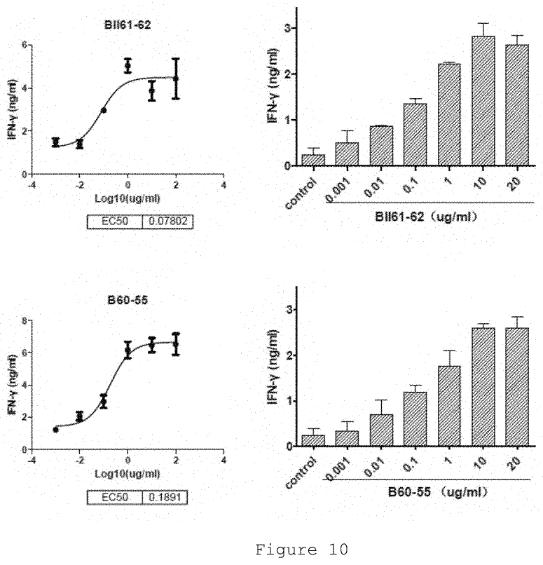

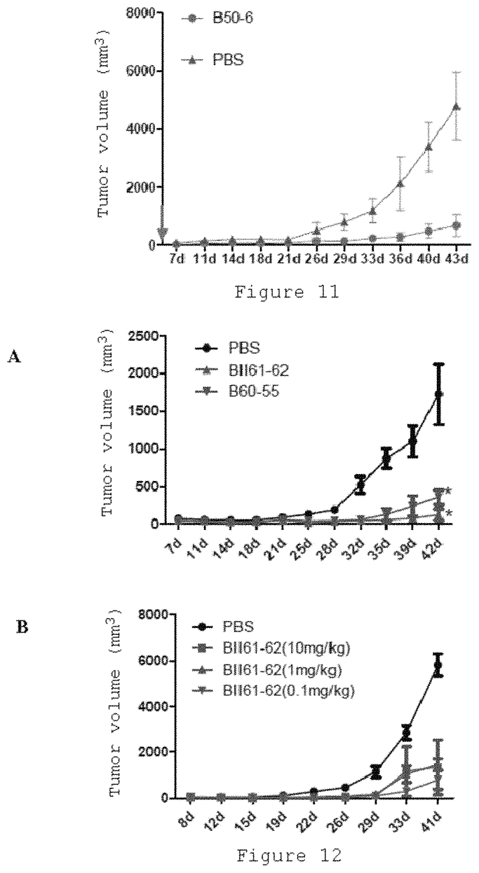

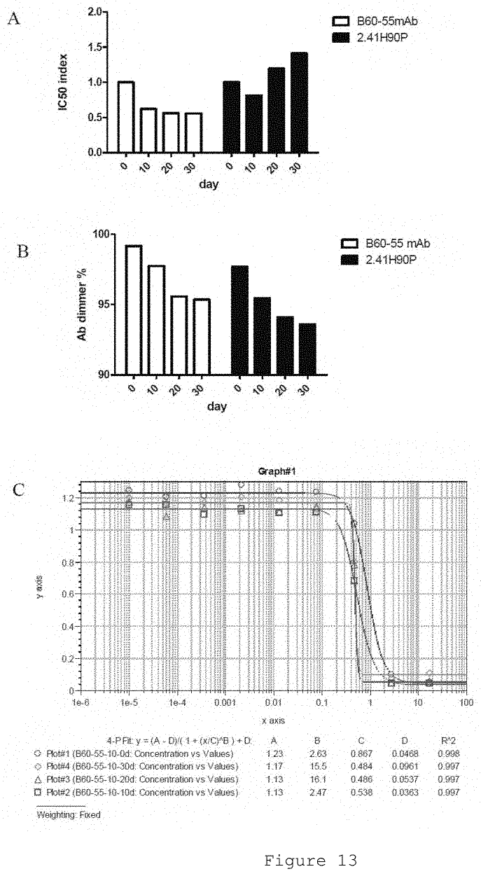

Immunodeficient NOD/SCID (non-obese diabetic/severe combined immunodeficiency) mice were used to study the in vivo activity of the PD-L1 antibodies B60-55 and BII61-62, which were incapable of recognizing murine PD-L1. Experiments using the melanoma cell line A375 (ATCC, CRL-1619™) which expresses human PD-L1 when subdermally transplanted into NOD/SCID mice and human peripheral blood mononuclear cells (PBMCs) were used to achieve the above objective. A375 cells and PBMCs were mixed at a ratio of 5:1 prior to injection and a subcutaneous injection with a total volume of 100 μl (containing 5 million A375 cells and 1 million PBMCs) was performed; antibodies were administered intraperitoneally on days 0, 7, 14, 21 and 28 following tumor inoculation (the antibody dose was 3 mg/kg for FIG. 11-A and the antibody doses are shown directly in FIG. 11 for FIG. 11-B), with PBS used as a negative control. Each experimental group consisted of 4-6 mice. Tumor formation was observed twice per week, dimensions were measured using Vernier calipers and the tumor volume was computed to draw a tumor growth curve (see FIG. 11); the results show that the antibodies B60-55 and BII-61-62 are capable of significantly inhibiting tumor growth.

### Example 9: A Comparison of the Stability of B60-55 and the Antibody 2.41H90P (Medimmune)

An accelerated stability test of the anti-PD-L1 antibody B60-55 and MedImmune LLC's antibody 2.41H90P was performed at 45° C. and the specific test procedure used was as follows: the anti-PD-L1 antibody B60-55 and MedImmune LLC's anti-PD-L1 antibody 2.41H90P (prepared according to the method for preparing 2.14H9 given in US Patent No. 20130034559, after which the antibody was renamed as 2.41H90P) were enriched to a concentration of 10 mg/ml, after which 100 μg of antibody was added to a 200 μg PCR tube and placed in a 45° C. batch; samples were taken on days 0, 10, 20 and 30, after which competitive ELISA and SE-HPLC analysis tests were performed, wherein the competitive ELISA method used was the same as that described in Example 6, to obtain IC50 values. SE-HPLC was performed using a Shimadzu LC LC20AT HPLC chromatograph; samples were concentrated to 1 mg/ml and samples were loaded at a flow rate of 0.5 ml/min, for a total sample volume of 50 μg; and isocratic elution was performed for 30 minutes following sample loading and the results shown in FIG. 12.

In FIG. 12, A shows a graphical comparison of IC50 values over time for B60-55 and the antibody 2.41H90P, and the data indicates that there were no significant changes in sample competitiveness at different time points; B shows the proportion of antibody dimers over time, and the data indicates that the dimer ratio decreased over time for both B60-55 and 2.41H90P; however, the rate at which 2.41H90P showed a decrease was faster than B60-55, indicating that B60-55 is more stable; and C shows the competitive ELISA curve obtained for B60-55 accelerated stability testing and the data show that B60-55 is capable of maintaining relatively good activity and stability.

### Example 10: Scaled Up Preparation and Formulation Stability of Antibody Variant B60-55-1

To evaluate potential for antibody preparation scale up, an example antibody variant B50-55-1 was cloned essentially as described in the foregoing disclosure. The amino acid sequence of B60-55-1 complete heavy chain was:

(SEQ ID NO: 85)
QVQLVQSGAEVKKPASSVKVSCTASGGSFSTYAISWVRQAPGQGLEWMGG
IIPIFGTTKYAQRFQGRVTITADESTTTAYMELSSLISDDTALYYCTTSR
GFNYGWFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVESCSVMHEALHNHYTQKSLSLS
PGK;

the corresponding DNA sequence was:

(SEQ ID NO: 86)
CAGGTCCAGCTTGTGCAGTCTGGAGCTGAGGTGAAGAAGCCTGCGTCCTC
GGTCAAAGTCTCCTGCACGGCTTCTGGCGGCTCCTTCAGCACCTATGCTA
TCAGTTGGGTGCGACAGGCTCCTGGACAAGGGCTTGAATGGATGGGCGGG
ATCATCCCCATCTTTGGTACAACTAAGTACGCACAGAGGTTCCAGGGCAG
GGTCACGATTACCGCGGACGAATCGACGACCACAGCCTACATGGAGCTGA
GCAGCCTGATATCTGACGACACGGCCCTGTATTATTGTACGACGTCTCGT
GGATTCAACTATGGCTGGTTTGACTACTGGGGCCAGGGTACCCTGGTCAC
CGTCTCCTCAGCCAGCACTAAGGGGCCCTCTGTGTTTCCACTCGCCCCTT
CTAGCAAAAGCACTTCCGGAGGCACTGCAGCACTCGGGTGTCTGGTCAAA
GATTATTTCCCTGAGCCAGTCACCGTGAGCTGGAACTCTGGCGCCCTCAC
CTCCGGGGTTCACACCTTTCCAGCCGTCCTGCAGTCCTCCGGCCTGTACT
CCCTGAGCAGCGTCGTTACCGTGCCATCCTCTTCTCTGGGGACCCAGACA
TACATCTGCAATGTCAACCATAAGCCTAGCAACACCAAGGTGGACAAAAA
GGTCGAGCCAAAGAGCTGCGATAAGACACACACCTGCCCTCCATGCCCCG
CACCTGAACTCCTGGGCGGGCCTTCCGTTTTCCTGTTTCCTCCCAAGCCC
AAGGATACACTGATGATTAGCCGCACCCCCGAAGTCACTTGCGTGGTGGT
GGATGTGAGCCATGAAGATCCAGAAGTTAAGTTTAACTGGTATGTGGACG
GGGTCGAGGTGCACAATGCTAAAACAAAGCCCAGGGAGGAGCAATATGCC
TCCACATACAGAGTGGTGTCCGTTCTGACAGTCCTGCACCAGGACTGGCT
GAACGGGAAGGAATACAAGTGCAAGGTGTCTAATAAGGCACTGCCAGCCC
CCATAGAGAAGACAATCTCTAAAGCTAAAGGCCAACCACGCGAGCCTCAG
GTCTACACACTGCCACCATCCAGGGAGGAAATGACCAAGAATCAGGTGAG
CCTGACTTGTCTCGTCAAAGGATTCTACCCAAGCGACATCGCCGTGGAGT
GGGAATCCAACGGCCAACCAGAGAACAACTACAAGACCACCCCACCAGTC
CTGGACTCTGATGGGAGCTTTTTCCTGTATTCCAAGCTGACAGTGGACAA
GTCTCGGTGGCAACAGGGCAACGTGTTCAGCTGCTCCGTGATGCATGAAG
CCCTGCATAACCACTATACCCAGAAAAGCCTCAGCCTGTCCCCCGGGAAA
TAATGA;

the amino acid sequence of the complete light chain was:

(SEQ ID NO: 87)
EIVMTQSPATLSLSPGERATLSCRASQSVGIHLAWYQQKPGQAPRLLIYG
ASSRATGIPDRFSGSGSGTDFTLTISRLEPEDFAVYYCQQYGSLPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;

the corresponding DNA sequence was:

(SEQ ID NO: 88)
GAAATTGTAATGACACAGTCTCCAGCCACCCTGTCTTTGTCTCCAGGGGA
AAGAGCCACCCTCTCCTGTAGGGCCAGTCAGAGTGTTGGCATACACTTAG
CCTGGTATCAACAGAAACCTGGCCAGGCTCCCAGGCTCCTCATCTATGGT
GCATCCAGTAGGGCCACTGGCATCCCAGACAGGTTCAGTGGCAGTGGGTC
TGGGACAGACTTCACTCTCACCATCAGCAGACTGGAGCCTGAAGATTTTG
CAGTGTATTACTGTCAGCAGTATGGTTCTTTACCTCGGACGTTCGGCCAA
GGGACCAAGGTGGAAATCAAACGTACGGTGGCTGCACCATCTGTCTTCAT
CTTCCCGCCATCTGATGAGCAGTTGAAATCTGGTACCGCTAGCGTTGTGT
GCCTGCTGAATAACTTTTATCCACGGGAGGCTAAGGTGCAGTGGAAAGTG
GACAATGCCCTCCAGAGCGGAAATAGCCAAGAGTCCGTTACCGAACAGGA
CTCTAAAGACTCTACATACTCCCTGTCCTCCACACTGACCCTCTCCAAGG
CCGACTATGAGAAACACAAGGTTTACGCATGCGAGGTCACACACCAGGGA
CTCTCCTCTCCCGTGACCAAGAGCTTCAACCGGGGAGAATGC;

B60-55-1 were produced in CHO cells grown in a bioreactor using either ActiCHO (GE) or Dynamis (Thermo Fisher Scientific) media. Initially, B60-55-1 were purified from clarified cell culture fluid using Protein A affinity chromatography resin MabSelect Sure LX, GE followed by two other chromatography steps—anion exchange chromatography on Q-adsorber (GE) membrane in a flow through mode and column chromatography on hydroxyapatite resin (CaPure-HA, Tosoh) which was the final polishing step.

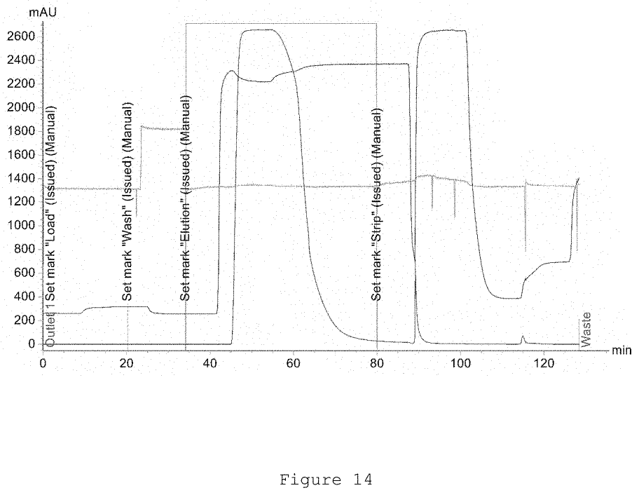

The observed step yield of B60-55-1 purification on the Protein A resin was about 95-98%. The observed step yield for Q-adsorber chromatography was about 93%-95%. The final purification step of B60-55-1 at which dimers, oligomers, and aggregates of B60-55-1, traces of residual DNA, and Protein A that leaks from Protein A column are removed is polishing chromatography on CaPure-HA which also serves as a good viral clearance step. The final hydroxyapatite step yield was about 77%-85%. Chromatogram of B60-55-1 purification on CaPure-HA is shown on FIG. 14.

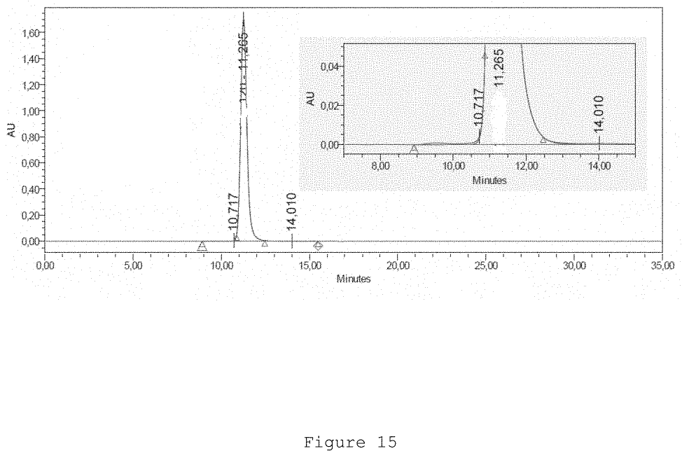

Homogeneity of B60-55-1 after chromatography on CaPure-HA, as assessed by size exclusion HPLC, was not lower than 99%. Analytical size exclusion chromatogram is shown on FIG. 15.

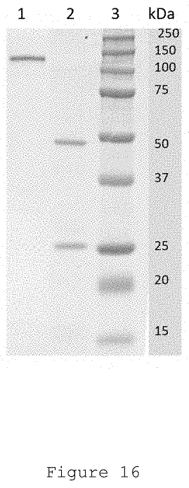

Polyacrylamide gel electrophoresis in the presence of SDS (SDS-PAGE) under reduced and non-reducing conditions also demonstrated high purity of B60-55-1 preparation. Images of Coomassie-stained gels are shown in FIG. 16.

LC-MS tryptic peptide mapping analysis of purified B50-55-1 showed that the heavy chain of the purified antibody is missing the C-terminal lysine residue, which does not affect the antigen biding properties of the purified antibody (see Example 11).

Several liquid formulations developed for B60-55-1 were tested in stressed stability studies. During these studies sterile samples of different B60-55-1 formulations with B60-55-1 at concentration of about 50 mg/mL were incubated at 40° C. for 6 weeks. Samples were pooled and analyzed at seven time points during the incubation: 0, 1 week, 2 weeks, 3 weeks, 4 weeks, 5 weeks, and 6 weeks. Subsequent testing was performed for pooled samples measuring protein concentration (concentration of B60-55-1), purity, integrity, turbidity, and osmolality. Protein concentration was measured by absorbance at 280 nm, protein identity and integrity were assessed by SDS-PAGE, turbidity was measured by A600, osmolality was measured by calibrated osmometer. Based on the results of the stresses stability experiments the following formulation was used for subsequent studies: 275 mM serine, 10 mM histidine, pH 5.9. In this formulation, after incubation at 40° C. for 5 weeks the purity of B60-55-1 exceeded 95%. Additionally, the following formulation produced substantially similar protein stability: 0.05% polysorbate 80, 1% D-mannitol, 120 mM L-proline, 100 mM L-serine, 10 mM L-histidine-HCl, pH 5.8.

### Example 11: Purified B60-55-1 and hPD-L1 Binding Kinetics Studies by SPR

The purpose of the study was comparative evaluation of binding parameters of B60-55-1 versus atezolizumab interaction with human PD-L1 using SPR method. The assay was carried out using several approaches and two versions of human PD-L1 were used, PD-L1-His tagged and PD-L1-Fc fusion protein. Series of different concentrations of PD-L1 ligands were used for calculating dissociation constants (Kd). The following equipment was utilized: R75000DC, plasmon resonance spectrometer, Reichert Technologies, Instrument #00478-1115 with SPRAutolink Control and TraceDrawer Evaluation Software packages. Sensor Chip SR7000 Gold Sensor Slide, 500 kDa Carboxymethyl dextran, Reichert, Inc, Prt No: 13206066

Following were the reagents used: B60-55-1 Stock Solution 32 mg/ml in 1% D-mannitol, 10 mM Na-Acetate, pH 5.4 with; atezolizumab (Tecentriq), 60 mg/ml, in 20 mM histidine, 14 mM acetic acid, 0.04% polysorbate 20, 4% sucrose, Lot 3109904, Genentech Inc; PD-L1-His tagged, Human recombinant, HEK293-derived, Phe19-Thr239, Accession #Q9NZQ7, R&D systems, Cat #9049-B7-100, Lot #DDIW0116081; PD-L1-Fc, human IgG Fc fusion protein, Human recombinant, HEK293-derived, Phe19-Thr239, Accession #Q9NZQ7, R&D systems, Cat #156-B7-100, Lot #DKL2116031; Human Antibody Capture Kit, GE Healthcare, Cat #BR-1008-39, Lot #10247121; Running buffer: lx PBS supplemented with 0.005% Tween-20, degassed and filtered through 0.2u filter.

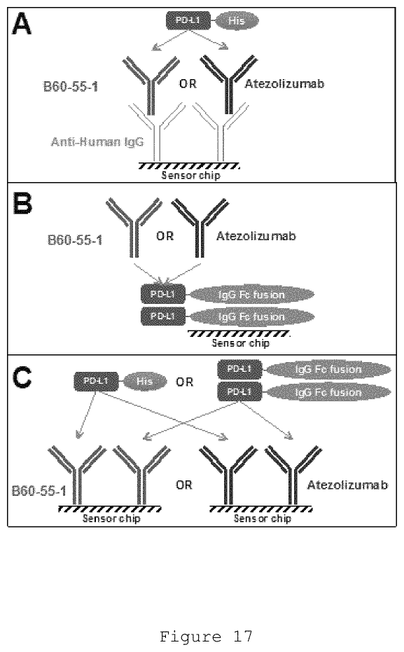

One of the standard approaches for measuring binding parameters is immobilization of capturing antibodies on a chip with subsequent loading of the test antibodies followed by ligand application. However, due to the presence on human IgG Fc fragment in PD-L1-Fc fusion protein, capturing mediated by anti-human antibodies could not be used for this ligand. Therefore, for PD-L1-Fc, an alternative approach was utilized illustrated in FIG. 17. For PD-L1-His tagged version of the ligand, antibody capturing approach illustrated in FIG. 17, panel A, was used. To test for binding of PD-L1-Fc fusion protein, two alternative methods were used: (1) direct immobilization of PD-L1-Fc itself illustrated in FIG. 17, panel B, and (2) immobilization of the test antibodies, B60-55-1 and comparator atezolizumab illustrated in FIG. 17, panel C.

All proteins were covalently attached to the chip using the same chemistry and protocol. The proteins conjugated to the chip included monoclonal anti-human IgG antibodies, PD-L1-Fc ligand, B60-55-1 and atezolizumab. Anti-human IgG and PD-L1-Fc were used in buffers compatible with the conjugation procedure whereas B60-55-1 and atezolizumab preparations were extensively dialyzed against 0.1×PBS before coupling. SR7000 Gold Sensor Slide was placed into the instrument and primed with Running Buffer, 1×PBS supplemented with 0.005% Tween 20, for 5 min at 250 μl/min, then allowed to stabilize at 25 μl/min. All steps were carried out at 25° C. Protein preparations were diluted using Immobilization Buffer (10 mM Na-acetate pH 5.0) to a final concentration of 25 μg/ml. Reagents for immobilization procedure were prepared as follows: EDC/NHS activation agent consisting of EDC (1-ethyl-3-(3-dimethylaminopropyl)-carbodiimide) at 40 mg/ml and NHS (N-hydroxysuccinimide) at 10 mg/ml in water, 1 M ethanolamine-HCl, pH 8.5 in water. Activation: EDC/NHS activation agent was injected into the chip at 10 μl/min for 8 min followed by 5 min wash with Running Buffer. Immobilization: anti-Human IgG at a final concentration of 25 μg/ml was injected into the chip at 10 μl/min for 8 min. Deactivation: unreacted active groups on the chip surface were blocked by injection of 1 M ethanolamine-HCl at 10 μl/min for 7 min. After antibody conjugation, the chip was washed with Running Buffer for 15 min at 25 μl/min.

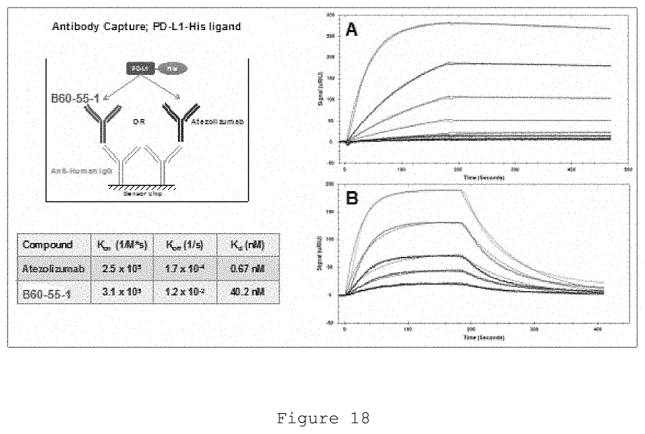

To study interaction of PD-L1-His tagged ligand with B60-55-1 and atezolizumab, antibody capturing approach was used. Anti-human IgG were covalently attached to the chip and used for capturing test antibodies as illustrated in FIG. 17, panel A. Chip with immobilized anti-human IgG was equilibrated with Running Buffer at a flow rate of 25 μl/min for 10-15 min. Test antibodies, B60-55-1 or atezolizumab, were loaded at 25 μl/min for 2 min, then the chip was washed for 3 min to remove unbound antibodies. PD-L1-His ligand 2-fold dilutions were prepared using Running Buffer starting from 100 nM concentration. Seven concentrations were used: 100, 50, 25, 12.5, 6.25, 3,125 and 1.56 nM. The ligand was loaded at 25 μl/min for 3 min. After ligand loading, dissociation phase of the experiment was carried out using Running Buffer at 25 μl/min flow rate for 5 min. Dissociation of protein complexes bound by immobilized anti-human IgG was carried out by 3 M MgCl₂ running though the chip at 25 μl/min for 30 sec. Series of sensograms for captured B60-55-1 or atezolizumab at different PD-L1-His ligand concentrations were generated as shown FIG. 18 and used for analysis. A kinetic evaluation of 1:1 binding model was used for the analysis of PD-L1-His interaction with the test antibodies. The following Kd values were obtained: B60-55-1 Kd=40.2 nM; atezolizumab Kd=0.67 nM

The results of study showed that the binding affinity of monomeric PD-L1 for the comparator atezolizumab was about 2-log higher than for B60-55-1, 0.67 nM vs. 40.2 nM, respectively. The lower affinity of B60-55-1-PD-L1-His interaction was due to a higher rate of dissociation, whereas the association phase for B60-55-1 and atezolizumab were essentially identical as follows from the table in FIG. 18.

To investigate the binding properties of PD-L1-Fc ligand with B60-55-1 and its comparator atezolizumab, PD-L1-Fc fusion protein was directly immobilized on the chip as illustrated in FIG. 17, panel B. To identify conditions for effective regeneration of the chip, scouting experiments were carried out. It was found that 3 M MgCl₂ did not dissociate bound antibodies (neither B60-55-1 nor atezolizumab) from immobilized PD-L1-Fc. Several regeneration conditions were tested including 10 mM glycine-HCl buffers with pH 3.0, pH 2.5, pH 2.0, and 10 mM NaOH. It was determined that pH 3.0 and pH 2.5 buffers did not effectively remove bound antibodies, whereas NaOH treatment inactivated ligand, resulting in loss of binding. It was subsequently concluded that glycine-HCl, pH 2.0 was suitable for these series of experiments.

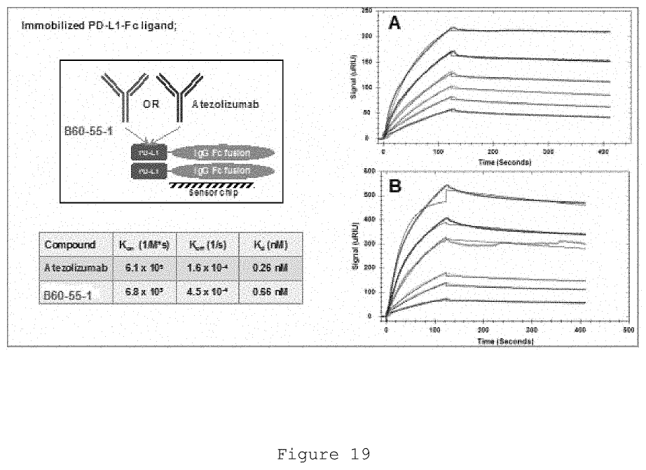

PD-L1-Fc ligand was immobilized on chip as described earlier in this example, and series of concentrations of B60-55-1 or atezolizumab were applied. Two-fold dilutions of B60-55-1 or atezolizumab were prepared using Running Buffer starting from 100 nM concentration. Seven concentrations were used: 100, 50, 25, 12.5, 6.25, 3,125 and 1.56 nM. The ligand was loaded at 25 μl/min for 3 min. After ligand loading, dissociation phase of the experiment was carried out using Running Buffer at 25 μl/min flow rate for 5 min. Series of sensograms for immobilized PD-L1-Fc at different concentrations of B60-55-1 or atezolizumab were generated (shown in FIG. 19) and used for analysis. A kinetic evaluation of 1:1 binding model was used for the analysis of immobilized PD-L1-Fc interactions with the test antibodies. The following Kd values were obtained: B60-55-1 Kd=0.66 nM; atezolizumab Kd=0.26 nM

The results of study showed that binding affinity of immobilized dimeric PD-L1-Fc were similar for B60-55-1 and for comparator atezolizumab, 0.6 nM vs. 0.26 nM, respectively as shown in the table in FIG. 19. The observed similarity of affinities of both antibodies reflects interactions with the dimeric ligand, which apparently were different from the interactions with the monomeric His-tagged version of the ligand.

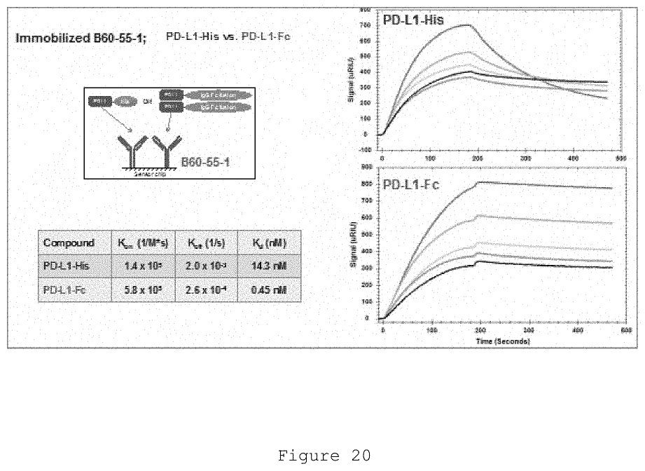

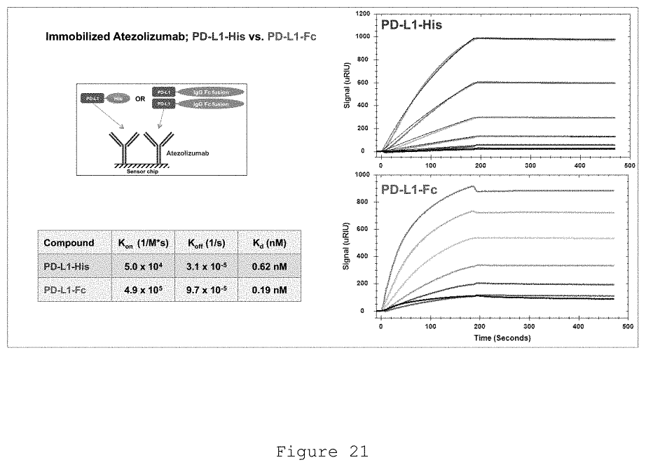

To further evaluate binding properties of the test antibodies, B60-55-1 or atezolizumab were covalently cross-linked on the chip as illustrated in FIG. 17, panel C. This approach enabled direct comparison of both versions of PD-L1 ligand, His-tagged and Fc-fusion proteins. Regeneration conditions of this binding system were re-evaluated and it was found that 10 mM glycine-HCl, pH 2.0 provided sufficient recovery. B60-55-1 and atezolizumab were immobilized on separate sensor chips as describe earlier in this example and various concentration of PD-L1-His or PD-L1-Fc fusion proteins were sequentially applied on immobilized antibodies. Two-fold dilutions of PD-L1-His or PD-L1-Fc were prepared using Running Buffer starting from 100 nM concentration. Seven concentrations were used: 100, 50, 25, 12.5, 6.25, 3,125 and 1.56 nM. The ligands were loaded at 25 μl/min for 3 min. After ligand loading, dissociation phase of the experiment was carried out using Running Buffer at 25 μl/min flow rate for 5 min. Series of sensograms for immobilized B60-55-1 or atezolizumab at different concentrations of PD-L1-His or PD-L1-Fc fusion protein were generated, as shown in FIGS. 20 and 21, and used for analysis. A kinetic evaluation of 1:1 binding model was used for the analysis of immobilized B60-55-1 interaction with both versions of ligands, PD-L1-His and PD-L1-Fc. The following Kd values were obtained for B60-55-1: for monomeric PD-L1-His ligand Kd=14.3 nM; for dimeric PD-L1-Fc ligand Kd=0.45 nM; for atezolizumab: for monomeric PD-L1-His ligand Kd=0.62 nM while for dimeric PD-L1-Fc ligand Kd=0.19 nM.

Thus, comparison of binding affinities of monomeric PD-L1-His and dimeric PD-L1-Fc to B60-55-1 and its comparator atezolizumab, revealed that B60-55-1 exhibits about 2-log higher affinity to PD-L1-Fc that to PD-L1-His, while atezolizumab has similar affinity towards PD-L1-His and PD-L1-Fc. The latter indicates that atezolizumab cannot distinguish between monomeric and dimeric forms of the ligand.

Evaluation of the binding properties of B60-55-1 and atezolizumab unexpectedly revealed B60-55-1 can substantially differentiate between a dimeric and a monomeric forms of its cognate target PD-L1, as opposed to a comparator antibody which is presently in clinical use.

### Example 12: Comparability of Effector Functions of B60-55-1 Antibody and Atezolizumab

This example discloses further analysis and comparison of the effector functions of B60-55-1 antibody with a comparator antibody atezolizumab. The present disclosure includes evaluations of binding to Fc gamma receptors: CD16a, CD32a, and CD64; antibody-dependent cell-mediated cytotoxicity (ADCC) activity using PD-L1 positive cells; complement-induced cytotoxicity (CDC) activity, C1q binding, and FcRn binding evaluations.

In addition to their role in binding antigen, antibodies can regulate immune responses through interacting with Fc gamma receptors via interactions with the Fc region of the antibody. These interactions with receptors present on natural killer (NK) and other myeloid cells, induce these cells to release cytokines such as IFNγ and cytotoxic granules containing perforin and granzymes, which culminates in ADCC.

The conducted studies revealed that B60-55-1 antibody exhibited no detectable binding to CD16a receptor while atezolizumab Kd for CD16a was 1.6E-5 M; B60-55-1 did not demonstrate detectable binding to CD32a receptor, while atezolizumab Kd for CD32a was 4.1E-5 M; B60-55-1 has a ten-fold lower binding to the CD64 receptors compared to other IgG1 antibodies, however it has a similar binding to CD64 as compared to atezolizumab.

Antibody-dependent cell-mediated cytotoxicity (ADCC) is a mechanism of action of antibodies through which virus-infected or other diseased cells are targeted for destruction by components of the cell-mediated immune system, such as natural killer cells. The ADDC reporter Bioassay Core Kit from Promega (Cat #G7014) is a bioluminescent reporter assay for quantifying ADCC. The assay combines effector cells expressing FcγRIIIa receptors on the cell surface that bind Fc fragments of test antibodies bound to the surface of the cells expressing the target receptor. The bridging of target cells to the effector cells through the biologic results in the activation of gene transcription through the NFAT pathway in effector cells, driving the expression of firefly luciferase, which can be quantified by luminescence. Since B60-55-1 did not show any binding to CD16a and CD32a, the molecule was not expected to demonstrate any ADCC activity. The assay was conducted using PD-L1 positive cell line A2058. The ADCC activity of B60-55-1 and atezolizumab was compared to ADCC of rituximab, an antibody known to exhibit strong ADCC activity.

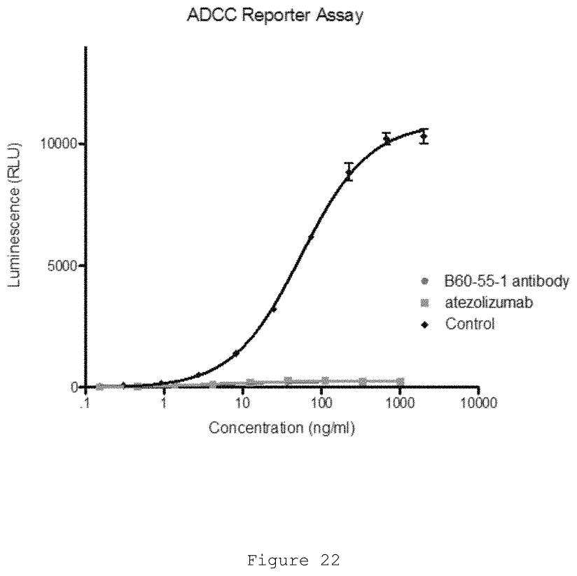

As expected for this engineered IgG1 antibody, B60-55-1 did not exhibit a substantial ADCC activity as compared to rituximab (control in FIG. 22), while it exhibited a comparable ADCC activity to atezolizumab.

B60-55-1 and atezolizumab are antibodies targeting PD-L1, the binding of both antibodies to C1q was compared. An antigen binding two-site ELISA was employed to examine the affinity with which both anti-PD-L1 antibodies interact with C1q. In this assay both antibodies were coated onto the plate at 25, 20, 15, 10, 8, 4, 2, 1, 0.5 and 0 μg/mL overnight at 4° C. The plate was then washed and blocked with SuperBlock solution, followed by addition of C1q (Sigma, Cat #C1740) at 2 μg/mL in binding buffer and incubated for 1 hour at room temperature. The plate was then washed and anti-C1q-HRP conjugate (Thermo, Cat. #PA1-84324) was added to the plate at 1:250 dilution in binding buffer (100 μL/well) for 1 hour at room temperature. Unbound HRP-conjugated antibody was removed by washing with Wash Buffer. The HRP activity was detected by using chromogenic substrate TMB. The color reaction was stopped by adding sulfuric acid, and the plate was read at 450 nm. Following a 3-parameter curve fit, EC50 was calculated for sample and for reference standard. The reportable value is % EC50 of reference standard EC50 relative to EC50 of sample, such that a higher % means a higher potency for the sample. The purpose of these experiments was to determine the binding of atezolizumab and B60-55-1 to C1q using ELISA format.

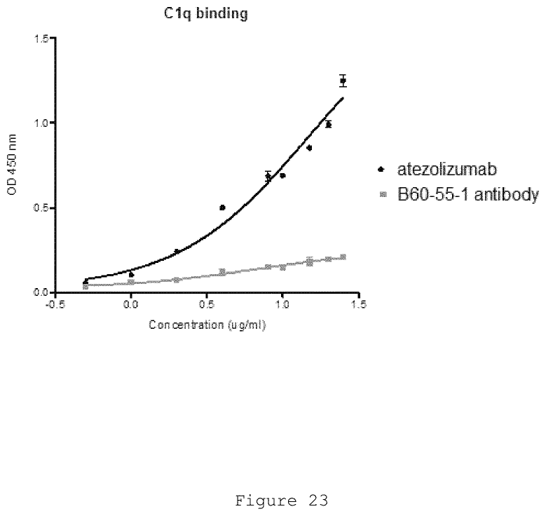

ELISA assay results are shown in FIG. 23. It was determined that EC-50 of atezolizumab binding to C1q was 14.9 μg/mL, while EC-50 of binding of B60-55-1 to C1q was 6.9 μg/mL. Therefore these binding characteristics are comparable.

Further, the ability to induce CDC on PD-L1 positive cells (A2058 cells) was compared between B60-55-1 and atezolizumab. In this assay cell lysis, ‘cell ghosts’ (lysed cells) can be observed microscopically and quantitated via the luminescent CytoTox-Glo reagent added to the cells for 1 hour.

Both products exhibited very low CDC activity. For atezolizumab EC50 was 0.09 μg/ml, while EC50 of B60-55-1 was 0.05 μg/ml.

IgG half-life is dependent on the neonatal Fc receptor (FcRn), which among other functions, protects IgG from catabolism. FcRn binds the Fc domain of IgG at an acidic pH ensuring that endocytosed IgG will not be degraded in lysosomal compartments and will then be released into the bloodstream. B60-55-1 and atezolizumab were compared for binding to FcRn receptor that was stably expressed by CHO cells.

The study showed that B60-55-1 binds to FcRn at Kd of 4.7e-7 M, which is typical for antibodies, while atezolizumab showed slightly higher affinity to FcRn with Kd of 1E-7 M.

### Example 13: Comparative Evaluation of Potencies of B60-55-1 Antibody, Atezolizumab and Pembrolizumab by Mixed Lymphocyte Reaction

Mixed lymphocyte reaction (MLR) assay was performed to evaluate the potencies of B60-55-1 and atezolizumab on T cell activation. The T cell activation was measured by the concentration of interleukin 2 (IL-2) secreted by T cells. Dendritic cells (DC) and CD4+ T cells were isolated from human Peripheral blood mononuclear cells (PBMC). The potency of pembrolizumab on T cell activation in MLR was used as the internal control to monitor the assay performance. Half maximal effective concentration (EC50) values were analyzed with the Sigmoidal dose-response non-linear regression fit by GraphPad Prism.

Reagents and Materials

RPMI 1640: Gibco, Invitrogen (Cat #22400); FBS, Gibco, (Cat #10099); Penicillin-Streptomycin (P/S): Gibco, Invitrogen (Cat #10378); Phosphate-Buffered Saline (PBS): Gibco, Invitrogen (Cat #10010-023); QC antibodies for dendritic cells: Anti-CD1a [HI149] (FITC), Abcam (ab18231), Anti-CD83 [HB15e] (FITC), Abcam (ab134491), Anti-CD86 [BU63] (FITC), Abcam (ab77276), Anti-HLA DR [GRB1] (FITC), Abcam (ab91335); CD4+ T Cell Isolation Kit: Miltenyi Biotec, (Cat #130-096-533); Pan Monocyte Isolation Kit: Miltenyi Biotec, (Cat #130-096-537).

Cell Line

Dendritic cells, prepared from freshly isolated human blood (over 20 healthy donors); CD4+ T cell, prepared from freshly isolated human blood (over 20 healthy donors).

Assay Kit

Human IL2 HTRF kit (Cisbio, Cat #64IL2PEB).

Detection Device

PHERAstarPlus, BMG Labtech.

Cell Preparation

CD4+ T cells were purificated by CD4+ T Cell Isolation Kit. PBMCs were prepared with density gradient centrifugation using Lymphoprep, the cells maintained in complete medium at 37° C./5% CO2 according to protocol from GenScript.

Dendritic cells were purificated by Pan Monocyte Isolation Kit. PBMCs were prepared with density gradient centrifugation using Lymphoprep, the cells maintained in complete medium at 37° C./5% CO2 according to protocol from GenScript. Purity of dendritic cells were validated by their surface markers by FACS (CD1a, CD83, CD86, and HLA-DR).

Antibodies Preparation

The samples were delivered in dry shipper and stored at 4° C. before testing. The samples were diluted with RPMI 1640 and applied to the tests.

Mixed Lymphocyte Reaction for Antibody Testing
Harvesting effector cells (CD4+ T cells) by centrifugation at 1000 rpm for 3 min;
Serial dilution of testing samples with assay buffer;
Seeding the effector cell stock to 96-well assay plate and add test sample;
Harvesting target cells (dendritic cells) by centrifugation at 1000 rpm for 3 min;
Adding the target cells to initiate the reaction and mix gently;
Incubating the plate at 37° C./5% CO2 incubator for 3 days;
Performing Human IL-2 test and read the plate;
Test concentration range for B60-55-1 and atezolizumab: starting from 300 nM, 3-fold dilution, 10 points in triplicates;
Test concentration range for pembrolizumab: Starting from 10 μg/ml, 5-fold dilution, 6 points in triplicates.

Mixed Lymphocyte Reaction (MLR) Assay

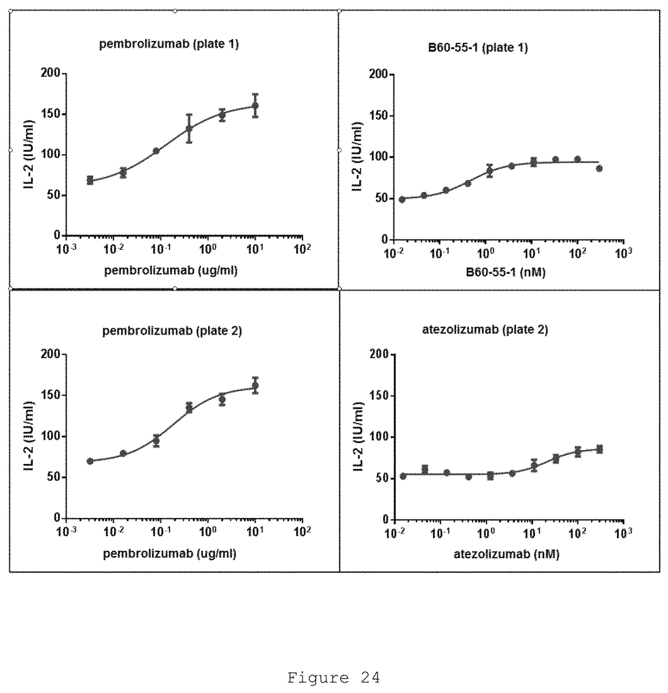

The results of the MLR assay are shown in FIG. 24, B60-55-1 and atezolizumab were able to activate T cells in MLR with different IL-2 secretions. The T cell activation data for pembrolizumab used as control was consistent with historic data. The analysis of the MLR data is shown in Table 2. The EC50 values for B60-55-1 and atezolizumab in the MLR assay were 0.4665 nM and 21.53 nM. Thus B60-55-1 activates T cells in the MLR assay with substantially higher potency.

TABLE 2
Best fit value summary for MLR

| | pembrolizumab | B60-55-1 | pembrolizumab | atezolizumab |
|---|---|---|---|---|
| Bottom | 60.62 | 49.49 | 68.18 | 55.2 |
| Top | 164.2 | 94.34 | 161.3 | 86.13 |
| LogEC₅₀ | −0.8871 | −0.3311 | −0.7364 | 1.333 |
| HillSlope | 0.7036 | 1.097 | 0.9005 | 1.356 |
| EC₅₀ | 0.1297 μg/ml | 0.4665 nM | 0.1835 μg/ml | 21.53 nM |
| EC₅₀ (nM) | 0.8705 | 0.4665 | 1.2315 | 21.53 |

### Example 14: Evaluation of B60-55-1 Efficacy in the Treatment of Subcutaneous MC38-hPD-L1 Murine Colon Carcinoma Model in Humanized PD-L1 Mice

The purpose of this study was testing in vivo efficacy of B60-55-1 and its comparator atezolizumab, both dosed at 10 mg/kg, in the treatment of subcutaneous MC38-hPD-L1 murine colon carcinoma engrafted into humanized PD-L1 mice.

Reagents and Equipment

Dulbecco's Modified Eagle's medium (DMEM): Cellgro, Catalog No. 10-013-CVR, stored at 4° C. Fetal Bovine Serum (FBS): Excell, Catalog No. FSP500, stored at −20° C. Phosphate buffer saline (PBS): Gibco, Catalog No. 20012027, stored at 4° C. Balance: Shanghai Shun Yu Heng Ping Science and Equipment Co. Ltd, Catalog No. MP5002. Caliper: Hexagon Metrolog, Catalog No. 00534220.

Test and Control Articles

Antibody B50-55-1 was stored in PBS at 50 mg/ml concentration; negative control IVIG: Guang Dong Shuang Lin BIO-Pharmacy Co. Ltd, Lot No 20160407, stored in PBS at 50 mg/ml; positive control antibody Atezolizumab: Genentech/Roche, Lot No 3109904, at 60 mg/ml was stored in a buffer containing glacial acetic acid (16.5 mg), L-histidine (62 mg), polysorbate 20 (8 mg) and sucrose (821.6 mg).

Dosing Solution Preparation

Test and control articles were diluted with PBS before dosing, stored at 2˜8° C. temporarily, and used at room temperature within 4 hours. Remaining test and control articles that had not been diluted were stored at 2˜8° C.

Animals

Forty male B-hPD-L1 humanized mice strain C57BL/6 were supplied by Beijing Biocytogen Co. Ltd. (quality certificate No.: 201716816)

Animal Housing Management

Animals were housed in a specific pathogen free barrier at Animal center of Beijing Biocytogen Co., Ltd. with 5 animals per individual ventilated cage (IVC). Animals were acclimated for three days to one week after arrival.

Temperature was maintained at 20-26° C. and humidity was maintained at 40-70%. Cages were made of polycarbonate, their size was 300 mm×180 mm×150 mm. The bedding material was pressure sterilized soft wood, which was changed once per week. The identification labels for each cage contained the following information: number of animals, sex, strain, date received, treatment, group number, and the starting date of the treatment. Animals had free access to autoclaved dry granule food and water during the entire study period. Food was SPF grade and purchased from Beijing Keao Xieli Feed Co., Ltd. Water was purified by ultrafiltration. Animals were marked by ear coding.

Experimental Methods and Procedures

The parental MC38 murine colon carcinoma cell line was purchased from Shunran Shanghai Biological Technology Co. Ltd. MC38-hPD-L1 cell line was constructed by replacing mouse PD-L1 with human PD-L1 by Biocytogen Co, Ltd. The cells were maintained in monolayer culture in DMEM supplemented with 10% heat inactivated FBS and were subcultured twice weekly. Cells growing in an exponential growth phase were harvested and counted for tumor inoculation.

Each mouse was subcutaneously injected with MC38-hPD-L1 tumor cells (5×10⁵) with 0.1 mL PBS in the right front flank for tumor development. Tumor-bearing animals were randomly enrolled into three study groups when the mean tumor size reached approximately 100 mm³. Each group consisted of eight mice. The test and control articles were administrated to the tumor-bearing mice according to predetermined regimens as shown below.

Dosing Regiment

| | | | | Working | | |
|---|---|---|---|---|---|---|
| | | No. of | Dosages | conc. | Dosing | |
| Groups | Treatment | animals | (mg/kg) | (mg/mL) | Route | Schedule |
| 1 | IVIG | 8 | 10 | 1 | i.p. | BIW × 8 |
| 2 | Positive | 8 | 10 | 1 | i.p. | BIW × 8 |
| | control | | | | | |
| 3 | B60-55-1 | 8 | 10 | 1 | i.p. | BIW × 8 |

Notes:
(1) Dosing volume was administrated based on body weight (10 μL/g).
(2) i.p. refers to intraperitoneal.
(3) BIW × 8 refers to a dosing frequency of twice a week and 8 times doses.

If body weight loss of a mouse exceeded 10%, treatment schedule was adjusted and dosing volume was reduced accordingly, alternatively the animal was suspended from the study.

After completing dosing, monitoring of the tumor volume and body weight was continued twice a week for up to 2 weeks.

Animals were euthanized with CO₂, followed by marrow breaking to confirm the euthanasia.

Tumor Measurements Index

Tumor size was measured twice weekly in two dimensions using a caliper, and the volume was expressed in mm³ using the formula: V=0.5 a×b² where a and b were the long and short diameters of the tumor, respectively.

Animals were weighed before tumor inoculation and animal grouping, then twice a week during the experiment, and finally before animals were euthanized at the end point of the experiment. Animals were weighed when accidental death happened or animals were on the verge of death.

During the entire period of the experiment, animals were checked twice a day (morning and afternoon) for their behavior and status, including but not limited to appearance of tumor ulcers, animal mental status, visual estimation of food and water consumption, and so on.

Tumors were collected and weighed at the time of study termination. Pictures were taken for both euthanized animals and collected tumors, and were attached in the report later.

Drug Evaluation Index

Relative tumor growth inhibition (TGI %): TGI %=(1−T/C)×100%. T and C refer to the mean relative tumor volume (RTV) of the treated and vehicle groups, respectively, on a given day. T/C % stands for the relative tumor proliferation rate^{[1]}, and the equation is: T/C %=T_{RTV}/C_{RTV}×100% (T_{RTV}: mean RTV of the treated groups; C_{RTV}: mean RTV of the vehicle group; RTV=Vₜ/V₀, V₀ refers to the tumor volume when grouping, Vₜ refers to the tumor volume measured at each indicated time points following treatment.)

Inhibition ratios of tumor weight (IR_{TW}%): At the endpoint, the tumors of animals were weighed, average tumor weight in each group was determined, and the IR_{TW}% was calculated by formula:

IR_{TW}%=(W_{control group}−W_{treatment group})/W_{control group}×100. W refers to the mean tumor weigh.

Data were analyzed using Student t-test/two way ANOVA, and P<0.05 was considered to be statistically significant. Both statistical analysis and biological observations were taken into consideration.

Results

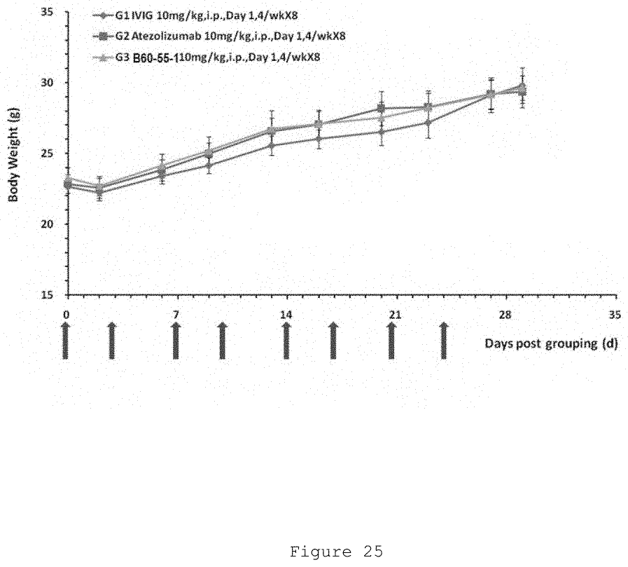

No obvious clinical signs were observed during the entire experiments. Body weights of most animals were gradually increased during the study. The mean body weight and mean percent body weight changes over time were shown in FIG. 25 and Table 3. Animals in B60-55-1 group showed no statistical difference on body weight compared with those in the control groups, (P>0.05).

TABLE 3
Body weight changes on humanized B-hPD-L1 mice with
murine colon cancer MC38-hPD-L1 tumor graft.

| | | | Body Weight | Body Weight | | Body |
|---|---|---|---|---|---|---|
| | | | (g) ^{a} | (g) ^{a} | | Weight |
| | | Animal | Before | 23 days post | | Change |
| Groups | Treatment | Number. | grouping | grouping | P^{b} | (g) |
| 1 | IVIG | 10 | 22.7 ± 0.5 | 27.2 ± 1.0 | — | +4.5 |
| 2 | Positive | 10 | 22.9 ± 0.7 | 28.3 ± 1.2 | 0.8 | +5.4 |
| | control | | | | | |
| 3 | B60-55-1 | 10 | 23.3 ± 0.7 | 28.2 ± 1.1 | 0.9 | +4.9 |

Note:
^{a} Mean ± SEM.
^{b}Statistical analysis via independent sample T-test on mean body weight of the treatment group versus vehicle group on day 23 post grouping.

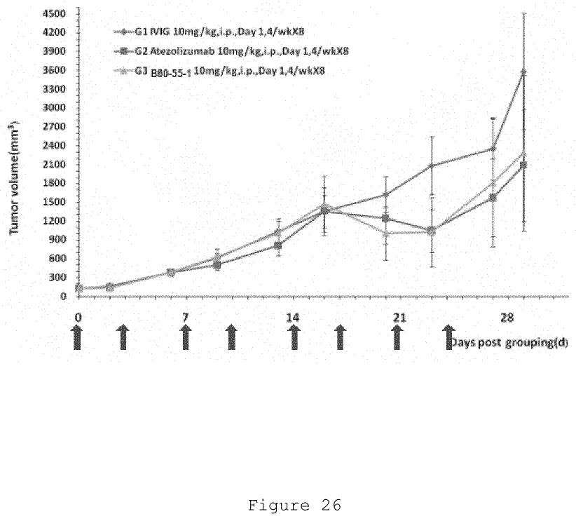

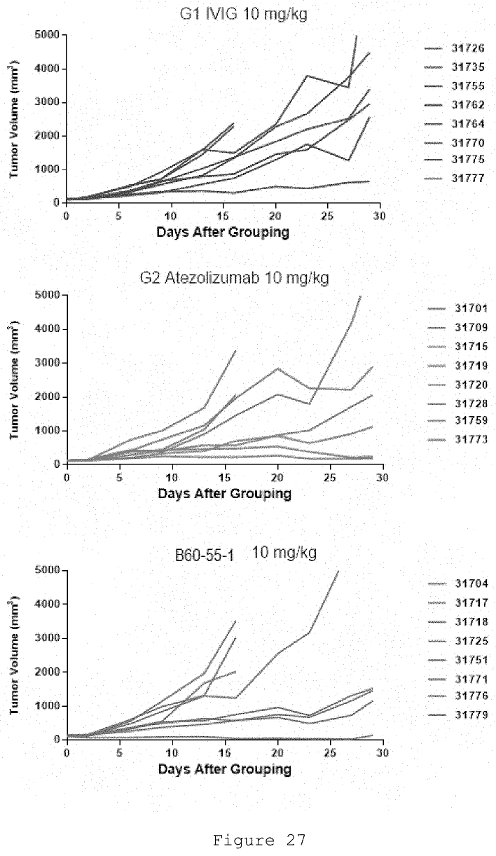

All mice were closely monitored for tumor growth during the entire experiment, with tumor size measured and recorded twice per week. The tumor growth inhibition (TGI %) was calculated and analyzed at the best therapeutic point (23 days post grouping). The statistical analysis results are shown in table 4 and 5. Individual mouse tumor growth in three groups were plotted in FIG. 26 and FIG. 27. Reduced tumor growth rate were both observed after atezolizumab and B60-55-1 administration. Distinct tumor regression in atezolizumab and B60-55-1 group was separately observed in 2/8 and 1/8 mice.

TABLE 4
Tumor growth inhibition of B60-55-1
on day 23 post grouping

| | | Tumor volume | | | |
|---|---|---|---|---|---|
| | | (mm³)^{a} | | | |
| | | | 23 days | | |
| | Animal | Before | after | TGI_{TV} | |
| Groups | number | grouping | grouping | (%) | p^{b} |
| G1:IVIG | 10 | 119 ± 4 | 2078 ± 459 | — | — |
| G2:Positive | 10 | 119 ± 4 | 1046 ± 336 | 52.7 | 0.10 |
| control | | | | | |
| G3:B60-55-1 | 10 | 120 ± 5 | 1022 ± 552 | 53.9 | 0.17 |

Note:
^{a}: Mean ± SEM.
^{b}: Statistical analysis via pooled standard deviation t-test on mean tumor volume of the treatment group versus vehicle group on day 23 post grouping.

TABLE 5
The statistical analysis of tumor volume
among various groups of B60-55-1

| Groups | 3 |
|---|---|
| 2: Positive | |
| control | 0.970 |
| 3: B60-55-1 | — |
| Note: | |

Statistical analysis via pooled standard deviation t-test on relative tumor volume on day 23 post grouping.

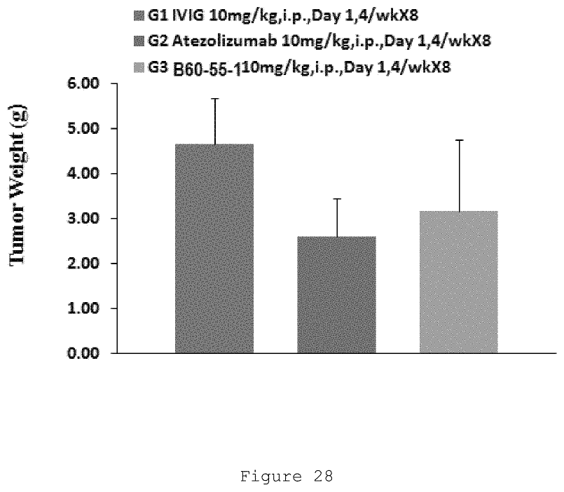

All tumors were dissected out from sacrificed mice, photographed and weighed on day 29 post grouping. The statistical analysis results of tumor weights are shown in Table 6 and FIG. 28. As the tumors were still growing after dosing completion, tumor growth inhibition rate (TGI_{TV}%) was compromised compared to that at day 23. Thus, tumor weights in treated groups at the endpoint of the study (day 23) had no significant differences from the vehicle group (P>0.05).

TABLE 6
Tumor weight inhibition of B60-55-1 on day 29 after
starting dosing

| | | | Tumor weight | |
|---|---|---|---|---|
| | Animal | Tumor weight | inhibition | |
| Groups | number | (g)^{a} | IR_{TW}% | p^{b} |
| G1: IVIG | 10 | 4.653 ± 1.009 | — | — |
| G2: Positive | 10 | 2.596 ± 0.860 | 44.2 | 0.193 |
| control | | | | |
| G3: B60-55-1 | 10 | 3.173 ± 1.570 | 31.8 | 0.447 |

Note:
^{a}: Mean ± SEM.
^{b}: Statistical analysis via independent sample T-test on mean tumor weight on day 29 post grouping

In this study body weights of most animals were gradually increased. Animals in B60-55-1 group showed no statistical difference on body weight compared with those in the control groups (P>0.05), indicating that B60-55-1 is safety at the present dosage. Reduced tumor growth rates were observed both after atezolizumab and B60-55-1 administration. At the best tumor growth inhibition point (day 23 post grouping), the mean tumor volume of vehicle control group was 2078±459 mm³, while in positive control treated groups, the mean tumor volume was 1046±336 mm³, and in the B60-55-1 treated groups, the mean tumor volume was 1022±552 mm³. The tumor growth inhibition TGI_{TV}% was 52.7% and 53.9%, respectively. At the endpoint of this study (day 29 post grouping), distinct tumor regression in atezolizumab and B60-55-1 group was observed in 2/8 and 1/8 mice, and tumor weight inhibition IR_{Tw}% was 44.2% and 31.8%, respectively. Comparing to the control group, tumor volumes of animals in B60-55-1 group the compound had anti-tumor activity but without significant difference.

Thus, in this study B60-55-1 showed comparable anti-tumor efficacy to atezolizumab at dose levels of 10 mg/kg without negatively affecting the animal body weight or inducing any abnormal clinical observations.

### Example 15: Evaluation of B60-55-1 in a Xenograft Model for Breast Cancer Using Humanized NSG™ Mice

Excluding skin cancer breast cancer is the most prevalent form of cancer in women, affecting about 7% of women by the time they reach 70 years of age (CDC). According to The American Cancer Society's estimates, there will be 252,710 newly diagnosed cases and 40,610 deaths in the United States in 2017. The 5-year relative survival rate during 2006-2012 was approximately 90% for all stages combined. Triple-negative breast cancer is a unique, aggressive subtype of breast cancer that is clinically negative for expression of estrogen and progesterone receptors and HER2 protein. Currently there are no targeted therapies to address this form of breast cancer. Developing a mouse model of primary human cancers is relevant to human disease as it represents a clinically relevant cancer model in mice that recapitulates the human disease. The Jackson Laboratory has established patient-derived xenograft (PDX) breast cancer models as well as cell line xenograft models in the highly immunodeficient NSG™ mouse strain as well as NSG™-derived strains such as NSG™-SGM3. The NSG™ (NOD.Cg-Prkdcscid Il2rgtm1Wjl/SzJ) mouse was developed for its ability to efficiently engraft human cells and tissues. Engraftment efficiency is significantly improved over other mouse strains due to the innate deficiencies in the immune system. Humanized NSG™ (hu-CD34 NSG™) mice are NSG™ mice injected with human CD34+ hematopoietic stem cells and have become important tools to study human immune function in vivo. These mice provide a strong preclinical platform for the application of novel immunotherapies, particularly those that are human specific and do not cross-react well with mouse. In addition, these models are used for genomic profiling of disease and/or for preclinical drug development. In this study, MDA-MB-231 cell line xenograft model for breast cancer established in humanized NSG™ mice was used to evaluate a novel antibody.

Mice and Housing

Female hu-CD34 NSG™ mice engrafted with human CD34+ cells that had >25% human CD45+ cells in the peripheral blood 16 weeks post engraftment were used for this study. Cohorts of hu-CD34 NSG™ mice engrafted with CD34+ cells from two donors were used. Mice were housed in individually ventilated polysulfone cages with HEPA filtered air at a density of up to 5 mice per cage. The animal room was lighted entirely with artificial fluorescent lighting, with a controlled 12 h light/dark cycle (6 am to 6 pm light). The normal temperature and relative humidity ranges in the animal rooms were 22-26° C. and 30-70%, respectively. The animal rooms were set to have up to 15 air exchanges per hour. Filtered tap water, acidified to a pH of 2.5 to 3.0, and standard rodent chow was provided ad libitum.

Methods and Records

Thirty eight (38) hu-CD34 NSG™ mice from two individual donors were implanted in the mammary fat pad with MDA-MB-231 cells at 5×106 in 1:1 mixture with Matrigel. Body weights and clinical observations were recorded 1×-2× weekly post implantation and digital caliper measurements were used to determine tumor volume 2× weekly once the tumors became palpable. Mice were randomized based on tumor volumes when the tumor volumes reached ˜62-98 mm3 and dosed according to Table 7 starting on Day 0. Body weights, clinical observations and digital caliper measurements were recorded 2× weekly post dose initiation. Animals that reached a body condition score of ≤2, a body weight loss of ≥20% or a tumor volume >2000 mm3 were euthanized before study terminus. Animals with ulcerated tumors were also euthanized before study terminus. All remaining animals were euthanized by CO2 asphyxiation on Study Day 41.

TABLE 7
Experimental Design

| | | | Dose | Dosing | Dosing |
|---|---|---|---|---|---|
| Group | N | Compound | (mg/kg) | Route** | Frequency*** |
| 1 | 10 | Vehicle* | N/A | IV | Twice |
| | | | (Volume | | weekly for |
| | | | equivalent) | | 6 weeks |
| 2 | 10 | Pembrolizumab | 10 | IV | Twice |
| | | | (on Day 0) | | weekly for |
| | | | 5 | | 6 weeks |
| | | | (thereafter) | | |
| 3 | 11 | B60-55-1 | 25 | IV | Twice |
| | | | | | weekly for |
| | | | | | 6 weeks |

*The same vehicle was used to formulate B60-55-1.
**The dosing route was switched to IP when it was not possible to do IV injection via tail vein due to swelling. One animal from Group 3 was dosed IP on Day 35, and one animal from Group 1 and two from Group 3 were dosed IP on Day 38.
***Animals were dosed on Days 0, 3, 7, 10, 14, 17, 21, 24, 28, 31, 35 and 38.

Results

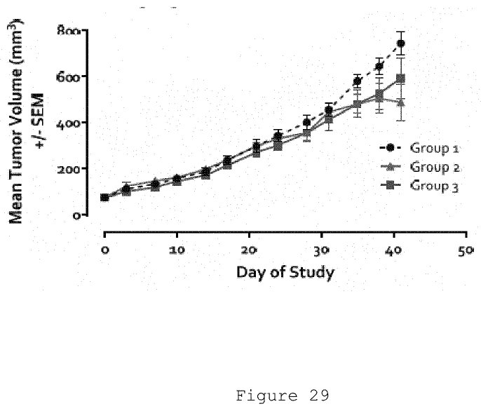

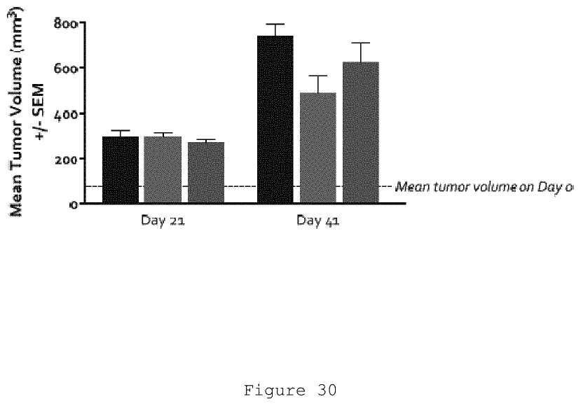

Results of the study are summarized in FIG. 29 and FIG. 30. The results indicate that the antibody B60-55-1 exhibits comparable efficacy to that of Pembrolizumab in the xenograft model for breast cancer used in the study.

Tecentriq is a registered trademark of Genentech USA, Inc.

Although specific embodiments of the present invention have been described here in detail, those skilled in the art will appreciate that it is possible to perform various modifications and substitutions of the more detailed aspects based on already published guidelines and teachings; and said changes all fall within the scope of the present invention. The full scope of the invention is given by the appended claims and any equivalent documentation.

1. An anti-PD-L1 antibody or an antigen-binding portion thereof, comprising groups of polypeptides selected from the group consisting of:
(1) a heavy chain comprising CDR1, CDR2 and CDR3 sequences which correspond to SEQ ID NO: 1, 2 and 3, respectively, and a light chain comprising CDR1, CDR2 and CDR3 sequences which correspond to SEQ ID NO: 4, 5 and 6, respectively;
(2) a heavy chain comprising CDR1, CDR2 and CDR3 sequences which correspond to SEQ ID NO: 7, 8 and 9, respectively, and a light chain comprising CDR1, CDR2 and CDR3 sequences which correspond to SEQ ID NO: 10, 11 and 12, respectively;
(3) a heavy chain comprising CDR1, CDR2 and CDR3 sequences which correspond to SEQ ID NO: 13, 14 and 15, respectively, and a light chain comprising CDR1, CDR2 and CDR3 sequences which correspond to SEQ ID NO: 16, 17 and 18, respectively;
(4) a heavy chain comprising CDR1, CDR2 and CDR3 sequences which correspond to SEQ ID NO: 1, 2 and 19, respectively, and a light chain comprising CDR1, CDR2 and CDR3 sequences which correspond to SEQ ID NO: 4, 5 and 6, respectively;
(5) a heavy chain comprising CDR1, CDR2 and CDR3 sequences which correspond to SEQ ID NO: 7, 20 and 9, respectively, and a 25 light chain comprising CDR1, CDR2 and CDR3 sequences which correspond to SEQ ID NO: 10, 11 and 12, respectively; and
(6) a heavy chain comprising CDR1, CDR2 and CDR3 sequences which correspond to SEQ ID NO: 13, 14 and 15, respectively, and a light chain comprising CDR1, CDR2 and CDR3 sequences which correspond to SEQ ID NO: 21, 17 and 18, respectively.

2. The anti-PD-L1 antibody or corresponding antigen-binding portion thereof as claimed in claim 1, comprising a heavy chain variable region having a sequence selected from among the following:
SEQ ID NO: 47, 49, 51, 53 or 54, or a sequence which is 70%, 80%, 85%, 90%, 95% or 99% identical to one of said sequences, respectively.

3. The anti-PD-L1 antibody or corresponding antigen-binding portion thereof as claimed in claim 1, comprising a light chain variable region having a sequence selected among the following:
SEQ ID NO: 48, 50, 52, 55 or 56, or a sequence which is 70%, 80%, 85%, 90%, 95% or 99% identical to one of said sequences, respectively.

4. The anti-PD-L1 antibody or corresponding antigen-binding portion thereof as claimed in claim 1, which corresponds to a whole antibody, bispecific antibody, scFv, Fab, Fab′, F(ab′)2 or Fv.

5. The anti-PD-L1 antibody or corresponding antigen-binding portion thereof as claimed in claim 4, which is a scFv further comprising a connecting peptide between the heavy chain and light chain variable regions.

6. The anti-PD-L1 antibody or corresponding antigen-binding portion thereof as claimed in claim 5, wherein said connecting peptide comprises a sequence of SEQ ID NO: 67.

7. The anti-PD-L1 antibody or corresponding antigen-binding portion thereof as claimed in claim 1, wherein the heavy chain constant region is selected from a group comprising IgG, IgM, IgE, IgD and IgA.

8. The anti-PD-L1 antibody or corresponding antigen-binding portion thereof as claimed in claim 7, wherein the heavy chain constant region is selected from a group comprising IgG1, IgG2, IgG3 and IgG4.

9. The anti-PD-L1 antibody or corresponding antigen-binding portion thereof as claimed in claim 1, wherein the light chain constant region is a κ region or A region.

10. A nucleic acid molecule, comprising a nucleic acid sequence capable of encoding an antibody heavy chain variable region, said antibody heavy chain variable region comprising a group of amino acid sequences selected from the group consisting of:
(i) SEQ ID NO: 1-3;
(ii) SEQ ID NO: 7-9;
(iii) SEQ ID NO: 13-15;
(iv) SEQ ID NO: 1, 2 and 19; and
(v) SEQ ID NO: 7, 20 and 9.

11. The nucleic acid molecule of claim 10, wherein said antibody heavy chain variable region comprises an amino acid sequence selected from among the following: SEQ ID NO: 47, SEQ ID NO: 49, SEQ ID NO: 51, SEQ ID NO: 53, and SEQ ID NO: 54.

12. A nucleic acid molecule, comprising a nucleic acid sequence capable of encoding an antibody light chain variable region, said antibody light chain variable region comprising a group of amino acid sequences selected from the group consisting of:
(i) SEQ ID NO: 4-6;
(ii) SEQ ID NO: 10-12;
(iii) SEQ ID NO: 16-18; and
(iv) SEQ ID NO: 21, 17 and 18.

13. The nucleic acid molecule of claim 12, wherein said antibody light chain variable region comprises an amino acid sequence selected from among the following:
SEQ ID NO: 48, SEQ ID NO: 50, SEQ ID NO: 52, SEQ ID NO: 55, SEQ ID NO: 56.

14. The nucleic acid molecule of claim 19, wherein the nucleic acid sequence is incorporated in a vector.

15. (canceled)

16. An anti-PD-L1 antibody, the antibody comprising a heavy chain having an amino acid sequence of SEQ ID NO: 85 and a light chain having an amino acid sequence of SEQ ID NO: 87, or an antigen-binding portion of the antibody.

17. A nucleic acid molecule, comprising a nucleic acid sequence capable of encoding a polypeptide having a sequence selected from the group consisting of:
SEQ ID NO: 85; and SEQ ID NO: 87.

18. The nucleic acid molecule of claim 17, said nucleic acid molecule comprising a sequence of SEQ ID NO: 86 or SEQ ID NO: 88.

19. The nucleic acid molecule of claim 17, wherein the nucleic acid molecule is present in a host cell.

20. The anti-PD-L1 antibody of claim 16, wherein the antibody is present in a composition that includes a pharmaceutically acceptable excipient or adjuvant.

21. The anti-PD-L1 antibody of claim 20, wherein the composition comprises about 275 mM serine, about 10 mM histidine, and has a pH of about 5.9.

22. The anti-PD-L1 antibody of claim 21, wherein the composition comprises about 0.05% polysorbate 80, about 1% D-mannitol, about 120 mM L-proline, about 100 mM L-serine, about 10 mM L-histidine-HCl, and has a pH of about 5.8.

23. A method of treating or preventing a disease or condition associated with modulation of activity of human PD-L1, the method comprising administering to a patient in need for treating or preventing a disease associated with modulation of activity of human PD-L1 a therapeutically effective amount of a pharmaceutical composition comprising an anti-PD-L1 antibody according to claim 16.

24. The method of claim 23, wherein said disease is a lung cancer, ovarian cancer, colon cancer, colorectal cancer, melanomas, kidney cancer, bladder cancer, breast cancer, liver cancer, lymphomas, hematologic malignancies, head and neck cancer, gliomas, gastric cancer, nasopharyngeal cancer, laryngeal cancer, cervical cancer, uterine cancer or osteosarcomas.

25. The method of claim 24, wherein said disease is a HBV, HCV or HIV infection.


SEQUENCE LISTING (87 sequences)

SEQ ID NO: 1 - protein, 5 aa, Artificial Sequence
  Synthetic sequence
TYAIS

SEQ ID NO: 2 - protein, 17 aa, Artificial Sequence
  Synthetic sequence
GIIPIFGTTKYAQRFQG

SEQ ID NO: 3 - protein, 11 aa, Artificial Sequence
  Synthetic sequence
SRGFSYGWFDY

SEQ ID NO: 4 - protein, 11 aa, Artificial Sequence
  Synthetic sequence
RASQSVGIHLA

SEQ ID NO: 5 - protein, 7 aa, Artificial Sequence
  Synthetic sequence
GASSRAT

SEQ ID NO: 6 - protein, 9 aa, Artificial Sequence
  Synthetic sequence
QQYGSLPRT

SEQ ID NO: 7 - protein, 7 aa, Artificial Sequence
  Synthetic sequence
SNSASWN

SEQ ID NO: 8 - protein, 18 aa, Artificial Sequence
  Synthetic sequence
RTYYRSKWYDDYAVSVKG

SEQ ID NO: 9 - protein, 13 aa, Artificial Sequence
  Synthetic sequence
SQGRYFVNYGMDV

SEQ ID NO: 10 - protein, 11 aa, Artificial Sequence
  Synthetic sequence
RASQSISSYLN

SEQ ID NO: 11 - protein, 7 aa, Artificial Sequence
  Synthetic sequence
GASSLQS

SEQ ID NO: 12 - protein, 11 aa, Artificial Sequence
  Synthetic sequence
QQSYFTPRGIT

SEQ ID NO: 13 - protein, 7 aa, Artificial Sequence
  Synthetic sequence
STKAAWY

SEQ ID NO: 14 - protein, 17 aa, Artificial Sequence
  Synthetic sequence
RTYFRSKWYNDYADSVK

SEQ ID NO: 15 - protein, 7 aa, Artificial Sequence
  Synthetic sequence
GQYTAFD

SEQ ID NO: 16 - protein, 14 aa, Artificial Sequence
  Synthetic sequence
TGTSSNVGGYDLVS

SEQ ID NO: 17 - protein, 7 aa, Artificial Sequence
  Synthetic sequence
EVIKRPS

SEQ ID NO: 18 - protein, 12 aa, Artificial Sequence
  Synthetic sequence
SSYAGRRLHGVF

SEQ ID NO: 19 - protein, 11 aa, Artificial Sequence
  Synthetic sequence
SRGFSYGWFDY

SEQ ID NO: 20 - protein, 18 aa, Artificial Sequence
  Synthetic sequence
RTYYRSKWYDDYAVSVKS

SEQ ID NO: 21 - protein, 14 aa, Artificial Sequence
  Synthetic sequence
TGTSSDVGGYDLVS

SEQ ID NO: 22 - protein, 30 aa, Artificial Sequence
  Synthetic sequence
QVQLVQSGAEVKKPASSVKVSCTASGGSFS

SEQ ID NO: 23 - protein, 14 aa, Artificial Sequence
  Synthetic sequence
WVRQAPGQGLEWMG

SEQ ID NO: 24 - protein, 32 aa, Artificial Sequence
  Synthetic sequence
RVTITADESTTTAYMELSSLISDDTALYYCTT

SEQ ID NO: 25 - protein, 11 aa, Artificial Sequence
  Synthetic sequence
WGQGTLVTVSS

SEQ ID NO: 26 - protein, 23 aa, Artificial Sequence
  Synthetic sequence
EIVMTQSPATLSLSPGERATLSC

SEQ ID NO: 27 - protein, 15 aa, Artificial Sequence
  Synthetic sequence
WYQQKLGQAPRLLIY

SEQ ID NO: 28 - protein, 32 aa, Artificial Sequence
  Synthetic sequence
GIPDRFSGSGSGTDFTLTISRLEPEDFAVYYC

SEQ ID NO: 29 - protein, 10 aa, Artificial Sequence
  Synthetic sequence
FGQGTKVEIK

SEQ ID NO: 30 - protein, 30 aa, Artificial Sequence
  Synthetic sequence
QVQLQQSGPGLVKPSQTLSLTCAISGDSVS

SEQ ID NO: 31 - protein, 14 aa, Artificial Sequence
  Synthetic sequence
WIRQSPSRGLEWLG

SEQ ID NO: 32 - protein, 32 aa, Artificial Sequence
  Synthetic sequence
RISINPDTSKNQFSLQLNSVTPEDTAVYYCAR

SEQ ID NO: 33 - protein, 11 aa, Artificial Sequence
  Synthetic sequence
WGQGTTVTVSS

SEQ ID NO: 34 - protein, 23 aa, Artificial Sequence
  Synthetic sequence
DIRLTQSPSSLSASVGDRITITC

SEQ ID NO: 35 - protein, 15 aa, Artificial Sequence
  Synthetic sequence
WYQQKPGKAPKLLVY

SEQ ID NO: 36 - protein, 32 aa, Artificial Sequence
  Synthetic sequence
GVPSRFSGSGSGTDFTLTISSLQPEDVATYYC

SEQ ID NO: 37 - protein, 10 aa, Artificial Sequence
  Synthetic sequence
FGPGTKVDIK

SEQ ID NO: 38 - protein, 30 aa, Artificial Sequence; identical to SEQ ID NO: 30
  Synthetic sequence

SEQ ID NO: 39 - protein, 14 aa, Artificial Sequence
  Synthetic sequence
WIRQSPSRGLEWLG

SEQ ID NO: 40 - protein, 33 aa, Artificial Sequence
  Synthetic sequence
SRLTINPDTSKNQFSLQLKSVSPEDTAVYYCAR

SEQ ID NO: 41 - protein, 12 aa, Artificial Sequence
  Synthetic sequence
IWGQGTMVTVSS

SEQ ID NO: 42 - protein, 22 aa, Artificial Sequence
  Synthetic sequence
QSALIQPASVSGSPGQSITISC

SEQ ID NO: 43 - protein, 15 aa, Artificial Sequence
  Synthetic sequence
WYQQYPGQAPRLIIY

SEQ ID NO: 44 - protein, 32 aa, Artificial Sequence
  Synthetic sequence
GISDRFSGSKSGNTASLTISGLQAEDEADYYC

SEQ ID NO: 45 - protein, 9 aa, Artificial Sequence
  Synthetic sequence
GGGTQLTVL

SEQ ID NO: 46 - protein, 15 aa, Artificial Sequence
  Synthetic sequence
WYQQKPGKAPKLLIY

SEQ ID NO: 47 - protein, 120 aa, Artificial Sequence
  Synthetic sequence
QVQLVQSGAEVKKPASSVKVSCTASGGSFSTYAISWVRQAPGQGLEWMGGIIPIFGTTKYAQRFQGRVTITADESTTTAYMELSSLISDDTALYYCTTSRGFNYGWFDYWGQGTLVTVSS

SEQ ID NO: 48 - protein, 107 aa, Artificial Sequence
  Synthetic sequence
EIVMTQSPATLSLSPGERATLSCRASQSVGIHLAWYQQKLGQAPRLLIYGASSRATGIPDRFSGSGSGTDFTLTISRLEPEDFAVYYCQQYGSLPRTFGQGTKVEIK

SEQ ID NO: 49 - protein, 125 aa, Artificial Sequence
  Synthetic sequence
QVQLQQSGPGLVKPSQTLSLTCAISGDSVSSNSASWNWIRQSPSRGLEWLGRTYYRSKWYDDYAVSVKGRISINPDTSKNQFSLQLNSVTPEDTAVYYCARSQGRYFVNYGMDVWGQGTTVTVSS

SEQ ID NO: 50 - protein, 109 aa, Artificial Sequence
  Synthetic sequence
DIRLTQSPSSLSASVGDRITITCRASQSISSYLNWYQQKPGKAPKLLVYGASSLQSGVPSRFSGSGSGTDFTLTISSLQPEDVATYYCQQSYFTPRGITFGPGTKVDIK

SEQ ID NO: 51 - protein, 120 aa, Artificial Sequence
  Synthetic sequence
QVQLQQSGPGLVKPSQTLSLTCAISGDSVSSTKAAWYWIRQSPSRGLEWLGRTYFRSKWYNDYADSVKSRLTINPDTSKNQFSLQLKSVSPEDTAVYYCARGQYTAFDIWGQGTMVTVSS

SEQ ID NO: 52 - protein, 120 aa, Artificial Sequence
  Synthetic sequence
QVQLVQSGAEVKKPASSVKVSCTASGGSFSTYAISWVRQAPGQGLEWMGGIIPIFGTTKYAQRFQGRVTITADESTTTAYMELSSLISDDTALYYCTTSRGFSYGWFDYWGQGTLVTVSS

SEQ ID NO: 53 - protein, 125 aa, Artificial Sequence
  Synthetic sequence
QVQLQQSGPGLVKPSQTLSLTCAISGDSVSSNSASWNWIRQSPSRGLEWLGRTYYRSKWYDDYAVSVKSRISINPDTSKNQFSLQLNSVTPEDTAVYYCARSQGRYFVNYGMDVWGQGTTVTVSS

SEQ ID NO: 54 - protein, 109 aa, Artificial Sequence
  Synthetic sequence
DIRLTQSPSSLSASVGDRITITCRASQSISSYLNWYQQKPGKAPKLLIYGASSLQSGVPSRFSGSGSGTDFTLTISSLQPEDVATYYCQQSYFTPRGITFGPGTKVDIK

SEQ ID NO: 55 - protein, 111 aa, Artificial Sequence
  Synthetic sequence
QSALIQPASVSGSPGQSITISCTGTSSDVGGYDLVSWYQQYPGQAPRLIIYEVIKRPSGISDRFSGSKSGNTASLTISGLQAEDEADYYCSSYAGRRLHGVFGGGTQLTVL

SEQ ID NO: 56 - DNA, 358 bp, Artificial Sequence
  Synthetic sequence
caggtccagcttgtgcagtctggagctgaggtgaagaagcctgcgtcctcggtcaaagtctcctgcacggcttctggcggctccttcagcacctatgctatcagttgggtgcgacaggctcctggacagggcttgaatggatgggcgggatcatccccatctttggtacaactaagtacgcacagaggttccagggcagggtcacgattaccgcggacgaatcgacgaccacagcctacatggagctgagcagctgatatctgacgacacggccctgtattattgtacgacgtctcgtggattcaactatggctggtttgactactggggccagggtaccctggtcaccgtctcctca

SEQ ID NO: 57 - DNA, 373 bp, Artificial Sequence
  Synthetic sequence
caggtacagctgcagcagtcaggtccaggactggtgaagccctcgcagaccctctcactcacctgtgccatctccggggacagtgtctctagcaacagtgcttcttggaactggatcaggcagtcccatcgagaggccttgagtggctgggaaggacatattacaggtccaaatggtatgatgattatgcagtatctgtgaaaggtcgaatcagcatcaacccagacacatccaagaaccagttctccctgcagtgaactctgtgactcccgaggacacggctgtgtattactgtgcaagaagccagggacgatattttgtcaactacggtatggacgtctggggccaagggaccacggtcaccgtctcctca

SEQ ID NO: 58 - DNA, 358 bp, Artificial Sequence
  Synthetic sequence
caggtacagctgcagcagtcaggtccaggactggtgaagccctcgcagaccctctcactcacctgtgccatctccggggacagtgtctctagcaccaaggctgcttggtactggatcaggcagtcccttcgagaggccttgagtggctgggaaggacatacttccggtccaagtggtataatgactatgccgactctgtgaaaagtcgattaaccatcaacccagacacatccaagaaccagttctccctgcaattaagtctgtgagtcccgaggacacggctgtgtattactgtgcaagagggcaatacactgcttttgatatctggggccaagggacaatggtcaccgtctcttca

SEQ ID NO: 59 - DNA, 358 bp, Artificial Sequence
  Synthetic sequence
caggtccagcttgtgcagtctggagctgaggtgaagaagcctgcgtcctcggtcaaagtctcctgcacggcttctggcggctccttcagcacctatgctatcagttgggtgcgacaggctcctggacagggcttgaatggatgggcgggatcatccccatctttggtacaactaagtacgcacagaggttccagggcagggtcacgattaccgcggacgaatcgacgaccacagcctacatggagctgagcagctgatatctgacgacacggccctgtattattgtacgacgtctcgtggattcagctatggctggtttgactactggggccagggtaccctggtcaccgtctcctca

SEQ ID NO: 60 - DNA, 373 bp, Artificial Sequence
  Synthetic sequence
caggtacagctgcagcagtcaggtccaggactggtgaagccctcgcagaccctctcactcacctgtgccatctccggggacagtgtctctagcaacagtgcttcttggaactggatcaggcagtcccatcgagaggccttgagtggctgggaaggacatattacaggtccaaatggtatgatgattatgcagtatctgtgaaaagtcgaatcagcatcaacccagacacatccaagaaccagttctccctgcagtgaactctgtgactcccgaggacacggctgtgtattactgtgcaagaagccagggacgatattttgtcaactacggtatggacgtctggggccaagggaccacggtcaccgtctcctca

SEQ ID NO: 61 - DNA, 315 bp, Artificial Sequence
  Synthetic sequence
gaaattgtaatgacacagtctccagccaccctgtctttgtctccaggggaaagagccaccctctcctgtagggccagtcagagtgttggcatacacttagcctggtaccaacagaaacttggccaggtcccaggctcctcatctatggtgcatccagtagggccactggcatcccagacaggttcagtggcagtgggtctgggacagacttcactctcaccatcagcagactggagcctgaagattttgcagtgattactgtcagcagtatggttctttacctcggacgttcggccaagggaccaaggtggaaat

SEQ ID NO: 62 - DNA, 325 bp, Artificial Sequence
  Synthetic sequence
gacatccggttgacccagtctccatcttccctgtctgcatctgtaggagacagaatcaccatcacttgccgggcaagtcagagcattagcagttatttaaattggtatcaacagaaaccagggaaagccctaagctcctggtctatggtgcatccagtttgcaaagtggggtcccatcaaggttcagtggcagtggatctgggacagatttcactctcaccatcagcagtctgcaacctgaagatgttgcaactactactgtcaacagagttactttaccccccgcgggatcactttcggccctgggaccaaagtggatatcaaa

SEQ ID NO: 63 - DNA, 332 bp, Artificial Sequence
  Synthetic sequence
cagtctgctctgattcagcctgcctccgtgtctgggtcccctggacagtcgatcactatctcctgtactggcaccagtagtaatgttggaggttatgaccttgtctcctggtaccaacagtacccgggccaagcccccagactcatcatttatgaggtcattaagcggccctcagggatttctgatcgcttctctggttccaagtctggcacacggcctccctgacaatctctgggctccaggctgaggacgaggctgattattattgcagctcatatgcaggtagacgtcttcatggtgtgttcggaggaggcacccagctgaccgtcctc

SEQ ID NO: 64 - DNA, 325 bp, Artificial Sequence
  Synthetic sequence
gacatccggttgacccagtctccatcttccctgtctgcatctgtaggagacagaatcaccatcacttgccgggcaagtcagagcattagcagttatttaaattggtatcaacagaaaccagggaaagccctaagctcctgatctatggtgcatccagtttgcaaagtggggtcccatcaaggttcagtggcagtggatctgggacagatttcactctcaccatcagcagtctgcaacctgaagatgttgcaactactactgtcaacagagttactttaccccccgcgggatcactttcggccctgggaccaaagtggatatcaaa

SEQ ID NO: 65 - DNA, 331 bp, Artificial Sequence
  Synthetic sequence
cagtctgctctgattcagcctgcctccgtgtctgggtcccctggacagtcgatcactatctcctgtactggcaccagtagtgatgttggaggttatgaccttgtctcctggtaccaacagtacccggccaagcccccagactcatcatttatgaggtcattaagcggccctcagggatttctgatcgcttctctggttccaagtctggcaacacggcctccctgacaatctctgggctccaggctgaggacgagctgattattattgcagctcatatgcaggtagacgtcttcatggtgtgttcggaggaggcacccagctgaccgtcctc

SEQ ID NO: 66 - protein, 15 aa, Artificial Sequence
  Synthetic sequence
GGGGSGGGGSGGGGS

SEQ ID NO: 67 - protein, 329 aa, Artificial Sequence
  Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - DNA, 989 bp, Artificial Sequence
  Synthetic sequence
gccagcactaaggggccctctgtgtttccactcgccccttctagcaaaagcacttccggaggcactgcagcactcgggtgtctggtcaaagattatttccctgagccagtcaccgtgagctggaacttggcgccctcacctccggggttcacacctttccagccgtcctgcagtcctccggcctgtactccctgagcagcgtcgttaccgtgccatcctcttctctggggacccagacatacatctgcaatgtcaccataagcctagcaacaccaaggtggacaaaaaggtcgagccaaagagctgcgataagacacacacctgccctccatgccccgcacctgaactcctgggcgggccttccgttttcctgtttcctccaagcccaaggatacactgatgattagccgcacccccgaagtcacttgcgtggtggtggatgtgagccatgaagatccagaagttaagtttaactggtatgtggacggggtcgaggtgcacaatgctaaacaaagcccagggaggagcaatataactccacatacagagtggtgtccgttctgacagtcctgcaccaggactggctgaacgggaaggaatacaagtgcaaggtgtctaataaggcactgccagccccatagagaagacaatctctaaagctaaaggccaaccacgcgagcctcaggtctacacactgccaccatccagggacgaactgaccaagaatcaggtgagcctgacttgtctcgtcaaaggattctaccaagcgacatcgccgtggagtgggaatccaacggccaaccagagaacaactacaagaccaccccaccagtcctggactctgatgggagctttttcctgtattccaagctgacagtggacaagtctcgtggcaacagggcaacgtgttcagctgctccgtgatgcatgaagccctgcataaccactatacccagaaaagcctcagcctgtcccccgggaaataatga

SEQ ID NO: 69 - protein, 107 aa, Artificial Sequence [sequence omitted]

SEQ ID NO: 70 - DNA, 319 bp, Artificial Sequence
  Synthetic sequence
cgtacggtggctgcaccatctgtcttcatcttcccgccatctgatgagcagttgaaatctggtaccgctagcgttgtgtgcctgctgaataacttttatccacgggaggctaaggtgcagtggaaagggacaatgccctccagagcggaaatagccaagagtccgttaccgaacaggactctaaagactctacatactccctgtcctccacactgaccctctccaaggccgactatgagaaacacaaggtttaccatgcgaggtcacacaccagggactctcctctcccgtgaccaagagcttcaaccggggagaatgc

SEQ ID NO: 71 - protein, 106 aa, Artificial Sequence
  Synthetic sequence
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 72 - DNA, 316 bp, Artificial Sequence
  Synthetic sequence
ggtcagcccaaggctgccccctcggtcactctgttcccaccctcctctgaggagcttcaagccaacaaggccacactggtgtgtctcgtaagtgacttctacccgggagccgtgacagtggcctggaggcagatggcagccccgtcaaggtgggagtggagaccaccaaaccctccaaacaaagcaacaacaagtatgcggccagcagctacctgagcctgacgcccgagcagtggaagtcccacagaagctacgctgccgggtcacgcatgaagggagcaccgtggagaagacagtggcccctgcagaatgctct

SEQ ID NO: 73 - DNA, 37 bp, Artificial Sequence
  Synthetic sequence
gcgcaagcttgccaccatgatcttcctcctgctaatg

SEQ ID NO: 74 - DNA, 31 bp, Artificial Sequence
  Synthetic sequence
gccgaattcgatagcactgttcacttccctc

SEQ ID NO: 75 - DNA, 37 bp, Artificial Sequence
  Synthetic sequence
gcgcaagcttgccaccatgctgcgtcggcggggcagc

SEQ ID NO: 76 - DNA, 34 bp, Artificial Sequence
  Synthetic sequence
gcgcgaattcggctatttcttgtccatcatcttc

SEQ ID NO: 77 - DNA, 21 bp, Artificial Sequence
  Synthetic sequence
gtacgagctaaaagtacagtg

SEQ ID NO: 78 - DNA, 20 bp, Artificial Sequence
  Synthetic sequence
tagatacccatacgacgttc

SEQ ID NO: 79 - DNA, 24 bp, Artificial Sequence
  Synthetic sequence
tctggtggtggtggttctgctagc

SEQ ID NO: 80 - DNA, 59 bp, Artificial Sequence
  Synthetic sequence
gccagatctcgagctattacaagtcttcttcagaaataagcttttgttctagaattccg

SEQ ID NO: 81 - DNA, 20 bp, Artificial Sequence
  Synthetic sequence
taatacgactcactataggg

SEQ ID NO: 82 - DNA, 24 bp, Artificial Sequence
  Synthetic sequence
ggcagccccataaacacacagtat

SEQ ID NO: 83 - DNA, 60 bp, Artificial Sequence
  Synthetic sequence
atggagacagacacactcctgctatgggtactgctgctctgggttccaggttccaccggt

SEQ ID NO: 84 - protein, 450 aa, Artificial Sequence
  Synthetic sequence
QVQLVQSGAEVKKPASSVKVSCTASGGSFSTYAISWVRQAPGQGLEWMGGIIPIFGTTKYAQRFQGRVTITADESTTTAYMELSSLISDDTALYYCTTSRGFNYGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1356 bp, Artificial Sequence
  Synthetic sequence
caggtccagcttgtgcagtctggagctgaggtgaagaagcctgcgtcctcggtcaaagtctcctgcacggcttctggcggctccttcagcacctatgctatcagttgggtgcgacaggctcctggacaagggcttgaatggatgggcgggatcatccccatctttggtacaactaagtacgcacagaggttccagggcagggtcacgattaccgcggacgaatcgacgaccacagcctacatggagctgagcagcctgatatctgacgacacggccctgtattattgtacgacgtctcgtggattcaactatggctggtttgactactggggccagggtaccctggtcaccgtctcctcagccagcactaaggggccctctgtgtttccactcgccccttctagcaaaagcacttccggaggcactgcagcactcgggtgtctggtcaaagattatttccctgagccagtcaccgtgagctggaactctggcgccctcacctccggggttcacacctttccagccgtcctgcagtcctccggcctgtactccctgagcagcgtcgttaccgtgccatcctcttctctggggacccagacatacatctgcaatgtcaaccataagcctagcaacaccaaggtggacaaaaaggtcgagccaaagagctgcgataagacacacacctgccctccatgccccgcacctgaactcctgggcgggccttccgttttcctgtttcctcccaagcccaaggatacactgatgattagccgcacccccgaagtcacttgcgtggtggtggatgtgagccatgaagatccagaagttaagtttaactggtatgtggacggggtcgaggtgcacaatgctaaaacaaagcccagggaggagcaatatgcctccacatacagagtggtgtccgttctgacagtcctgcaccaggactggctgaacgggaaggaatacaagtgcaaggtgtctaataaggcactgccagcccccatagagaagacaatctctaaagctaaaggccaaccacgcgagcctcaggtctacacactgccaccatccagggaggaaatgaccaagaatcaggtgagcctgacttgtctcgtcaaaggattctacccaagcgacatcgccgtggagtgggaatccaacggccaaccagagaacaactacaagaccaccccaccagtcctggactctgatgggagctttttcctgtattccaagctgacagtggacaagtctcggtggcaacagggcaacgtgttcagctgctccgtgatgcatgaagccctgcataaccactatacccagaaaagcctcagcctgtcccccgggaaataatga

SEQ ID NO: 86 - protein, 214 aa, Artificial Sequence
  Synthetic sequence
EIVMTQSPATLSLSPGERATLSCRASQSVGIHLAWYQQKPGQAPRLLIYGASSRATGIPDRFSGSGSGTDFTLTISRLEPEDFAVYYCQQYGSLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 642 bp, Artificial Sequence
  Synthetic sequence
gaaattgtaatgacacagtctccagccaccctgtctttgtctccaggggaaagagccaccctctcctgtagggccagtcagagtgttggcatacacttagcctggtatcaacagaaacctggccaggctcccaggctcctcatctatggtgcatccagtagggccactggcatcccagacaggttcagtggcagtgggtctgggacagacttcactctcaccatcagcagactggagcctgaagattttgcagtgtattactgtcagcagtatggttctttacctcggacgttcggccaagggaccaaggtggaaatcaaacgtacggtggctgcaccatctgtcttcatcttcccgccatctgatgagcagttgaaatctggtaccgctagcgttgtgtgcctgctgaataacttttatccacgggaggctaaggtgcagtggaaagtggacaatgccctccagagcggaaatagccaagagtccgttaccgaacaggactctaaagactctacatactccctgtcctccacactgaccctctccaaggccgactatgagaaacacaaggtttacgcatgcgaggtcacacaccagggactctcctctcccgtgaccaagagcttcaaccggggagaatgc